(19) United States
(12) Patent Application Publication
(10) Pub. No.: US 2023/0270883 A1
(43) Pub. Date: Aug. 31, 2023

(54) ACTIVE AGENT RELEASE PARTICLE

(71) Applicant: Oxford University Innovation Limited, Oxtord (GB)

(72) Inventors: Helen Elizabeth TOWNLEY, Oxford (GB); Benjamin WHITE, Begbroke (Yarnton) (GB)

(21) Appl. No.: 18/020,525

(22) PCT Filed: Aug. 13, 2021
(86) PCT No.: PCT/GB2021/052104

§ 371 Date: Feb. 9, 2023

(30) Foreign Application Priority Data
Aug. 14, 2020 (GB) 2012717.1

(51) Int. Cl. A61K 47/69; A61K 47/64; A61K 47/58; C08F 220/56

(52) U.S. Cl. CPC A61K 47/6929; A61K 47/643; A61K 47/58; C08F 220/56

(57) ABSTRACT

The present invention concerns a complex comprising an active agent, a polymer and an iron oxide nanoparticle. The complex may also comprise an active agent. Also described are methods of releasing an active agent from the complex, for instance by irradiating the complex with radio waves. Also described are compositions and articles comprising the complex. Also described are methods of therapy and particularly methods of treatment of cancer involving the complex described herein. In addition, the invention concerns a polymer which is particularly useful in preparing the complex described herein. The polymer is capable of undergoing a phase change at a predetermined temperature, such as 39-42° C. In one embodiment, the polymer is a copolymer of N-isopropylacrylamide, acrylic acid and acrolein. In another embodiment, the polymer is a copolymer of N-isopropylacrylamide acrylamide and allyl mercaptan.

### FIELD OF THE INVENTION

The invention relates to a nanoparticle-polymer complex comprising an iron oxide nanoparticle bound to a polymer capable of undergoing a phase change at a predetermined temperature. The invention also relates to methods of use of the nanoparticle-polymer complex to release an active agent, and the use of such methods in therapy (for instance the treatment of cancer) and other processes. The invention further relates to a novel polymer which can be used to prepare such nanoparticle-polymer complexes.

### BACKGROUND TO THE INVENTION

For the last two decades, the synthesis and properties of superparamagnetic and ferrimagnetic iron oxide nanoparticles (ION) have been intensively studied. In nature, iron oxide is found as a mineral called magnetite, which exhibits permanent magnetism.

In 1993 it was shown that superparamagnetic crystal suspensions (such as suspensions of iron oxide nanoparticles) can absorb energy from an oscillating magnetic field and convert it into thermal energy. Similarly, iron oxide nanoparticles in solution can absorb low and medium radio frequencies of electromagnetic radiation as thermal energy. The conversion of magnetic energy to thermal energy can occur by various mechanisms, discussed in detail in “Magnetic nanoparticle-based therapeutic treatments for thermo-chemotherapy treatment of cancer”, Nanoscale, 2014, 6, 11553. Since the majority of tissues in the body have no absorption in the radio wave spectrum, internal heating can be caused at a specific site within the body by heating such nanoparticles in vivo. Accordingly, this method has many potential biomedical applications as well as non-biological applications.

In particular, iron oxide and other magnetic nanoparticles in vivo have been used to increase the local temperature of tumour tissue in patients and hence to destroy cancerous cells. This method of treatment is referred to as “hyperthermia”. Hyperthermia has proved to be a very effective and discriminating treatment since cancerous cells are more sensitive to changes in temperature than healthy cells.

Magnetic nanoparticles have a further advantageous property: they can be guided to a particular location using a magnetic field. For instance, magnetic nanoparticles within a bloodstream of a patient can be guided to a particular location in the patient's body by a magnet or magnets. Thus, magnetic nanoparticles can be guided to a desired location (for instance to a tumour) before a hyperthermic treatment is carried out in a patient.

Recently, it has been suggested that magnetic nanoparticles may be combined with drugs in a single formulation, to provide a tool capable of providing hyperthermia treatment and delivery of the drug at a specific location (“Magnetic nanoparticle-based therapeutic treatments for thermo-chemotherapy treatment of cancer”, Nanoscale, 2014, 6, 11553). However, difficulties have been encountered in providing a formulation which retains the drug and transports it to the treatment site, and effectively releases the drug at the treatment site.

Thermo-responsive polymers, also called a thermosensitive polymers or smart polymers, undergo a conformational change at a specific temperature, called the lower critical solution temperature (LCST). At temperatures below the LCST, the thermo-responsive polymer adopts an expanded structure which is well-solvated by water molecules. This structure is regarded as a first “phase”. However, upon heating, the thermo-responsive polymer undergoes a reversible lower critical solution temperature phase transition and adopts a different phase. The phase adopted above the LCST is a hydrophobic, globular conformation.

The thermodynamic driving force behind the phase change can be explained by the Gibbs equation:

ΔG=ΔH−TΔS (1)

For any reaction to occur, the Gibbs free energy change (ΔG) for the reaction must have a negative value. At temperatures below the LCST, ΔG is dominated by the enthalpy change of hydration (ΔH) which has a large negative value due to the exothermic hydrogen bonding between the polymer and water molecules. When the polymer is heated above the LCST, the entropy term (TΔS) increases and it becomes thermodynamically favourable to release water bound to the polymer. Hence, with rising temperatures the structure or phase adopted by the thermo-responsive polymer changes from expanded to globular. Thermo-responsive polymers are often described as having inverse solubility, having a greater solubility in water at lower temperatures.

An example of a thermo-responsive polymer is poly N-isopropylacrylamide, PNIPAM:

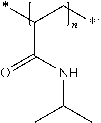

PNIPAM has a LCST of 32° C. In systems such as the human body, which has a characteristic temperature of around 37° C., PNIPAM assumes its globular phase immediately.

### SUMMARY OF THE INVENTION

The inventors have appreciated that thermo-responsive polymers may be combined with iron oxide nanoparticles to provide a system which is capable of retaining and transporting an active agent within a system and then, upon remote heating, delivering the active agent at a desired position. The inventors have provided a complex which combines the advantages of iron oxide nanoparticles (which can be remotely manipulated and heated) with the ability of thermo-responsive polymers to undergo a phase change from an expanded to a globular conformation at a particular temperature. In particular, the inventors have appreciated that remote heating of iron oxide nanoparticles can be used to locally heat a complex, comprising an iron oxide nanoparticle and a thermo-responsive polymer bound thereto, to above a particular pre-determined temperature. This causes the thermo-responsive polymer to undergo a phase change from a solvated (expanded) phase to a condensed or globular phase. If present, an active agent within the thermo-responsive polymer will be released from the thermo-responsive polymer. The iron oxide nanoparticles and the polymer bound thereto can then be removed from the system.

Accordingly, the invention provides nanoparticle-polymer complex comprising an iron oxide nanoparticle bound to a polymer capable of undergoing a phase change at a predetermined temperature. Typically, the nanoparticle-polymer complex also comprises an active agent. The pre-determined temperature may be above the ambient temperature of the system (for instance above human body temperature) to ensure that the active agent is released upon heating, and not before.

Generally, to improve the loading of the complex with an active agent and to ensure the active agent does not diffuse out of the complex below the predetermined temperature, the complex typically also comprises a carrier to attach the active agent to the complex. Accordingly, the invention provides a nanoparticle-polymer complex which further comprises a carrier immobilised on the polymer. The carrier may also provide other useful functions such as stabilising the active agent and carrying it into solution. For instance, where the active agent has a low hydrophilicity, it is useful to bind it to a hydrophilic carrier to carry it into aqueous solution. Accordingly, the complex may comprise a carrier which is reversibly covalently bound to the polymer. An active agent may be bound to the carrier.

It is desirable therefore to employ a thermo-responsive polymer in the invention which is modified to be able to bind to an active agent or a carrier. Accordingly, the polymer generally comprises a “binding unit”, which can bind a carrier or active agent. However, the inventors have found that when conventional thermo-responsive polymers such as PNIPAM are modified to include a binding unit, the temperature at which they will undergo a phase change (the “lower critical solution temperature”, or LCST) is affected. The inventors have found that the LCST may be adjusted to a desired pre-determined temperature by inclusion of another repeating unit in the polymer: a LCST-adjusting unit, which is typically hydrophilic. Accordingly, in some embodiments, the nanoparticle-polymer complex of the invention comprises a polymer which comprises the following repeating units: a phase-change unit, a binding unit and an LCST-adjusting unit.

The nanoparticle-polymer complex of the invention could be used in in the gas phase but, in practice, will typically be present in solution for ease of use. Accordingly, the invention provides a composition comprising the nanoparticle-polymer complex of the invention. The composition may comprise suitable excipients depending on the intended use of the complex.

In some circumstances, it may be desired to provide two different active agents (such as vitamin C and vitamin K3) at a particular location. However, some active agents (for instance, vitamin C and vitamin K3) must be kept apart prior to administration to prevent their interaction. This problem can conveniently be addressed by a composition according to the invention comprising a mixture of nanoparticle-polymer complexes containing each active agent separately. Accordingly, the invention provides a composition containing two or more nanoparticle-polymer complexes wherein a first nanoparticle-polymer complex comprises a first active agent and the second nanoparticle-polymer complex comprises a second active agent.

The invention further provides an article comprising the nanoparticle-polymer complex of the invention. The article may contain or may be coated with the nanoparticle-polymer complex of the invention. For instance, the invention provides a stent comprising the nanoparticle-polymer complex.

The nanoparticle-polymer complex described herein can be used to release an active agent by exploiting the fact that iron oxide nanoparticles can be heated remotely when subjected to an alternating magnetic field. Accordingly, the invention provides a method of releasing an active agent, which method comprises:
a) providing a nanoparticle-polymer complex comprising an active agent and an iron oxide nanoparticle bound to a polymer capable of undergoing a phase change at a predetermined temperature; and
b) exposing the nanoparticle-polymer complex to an alternating magnetic field and thereby heating the iron oxide nanoparticle, causing the phase change to occur and active agent to be released.

This method is useful in any situation where it is desired to release an active agent at a specific locus within a system. As the complex can be heated remotely, the active agent can be released at the desired location without the need to insert the active agent at the active site. This is useful in systems which contain sealed pipework. It is also particularly useful in treatment of the human or animal body. Accordingly, the invention provides a method of treatment or prevention of a disorder, the method comprising
a) administering the nanoparticle-polymer complex comprising an iron oxide nanoparticle, an active agent and a polymer capable of undergoing a phase change at a predetermined temperature to a subject in need thereof; and
b) exposing the nanoparticle-polymer complex to an alternating magnetic field and thereby heating the iron oxide nanoparticle, causing the phase change to occur and active agent to be released.

This method is particularly useful as it can supply active agent (e.g. a chemotherapeutic agent) at a tumour site and so the disorder is preferably cancer. Generally the cancer is a solid cancer, rather than a blood cancer.

The invention also provides a composition for use in a method of treatment or prevention of a disorder as described herein, wherein the composition comprises a nanoparticle-polymer complex as described herein.

The invention also provides the use of a nanoparticle-polymer complex comprising an active agent, as described herein, to release an active agent. Typically said use is use in a method as described herein.

As described above, the invention provides a nanoparticle-polymer complex comprising a variety of polymers, the pre-determined temperatures of which are adjusted according to the particular intended use. The inventors have developed a particular class of thermosensitive polymers which is particularly suitable for manufacturing nanoparticle-polymer complexes which are intended to release an active agent upon heating in a human or animal body. These polymers can have LCST values in excess of 40° C., particularly in excess of 41° C., meaning that they will not undergo a phase change at normal body temperatures in the region of 37° C. However, on heating above the LCST, they will undergo a phase change and can release an active agent bound thereto. Thus, such polymers are useful for controlled release of an active agent in the body. Accordingly, the invention also provides a thermosensitive polymer comprising a phase change repeating unit of formula (XA), a binding repeating unit of formula (YE) and a hydrophilic repeating unit of formula (ZB):

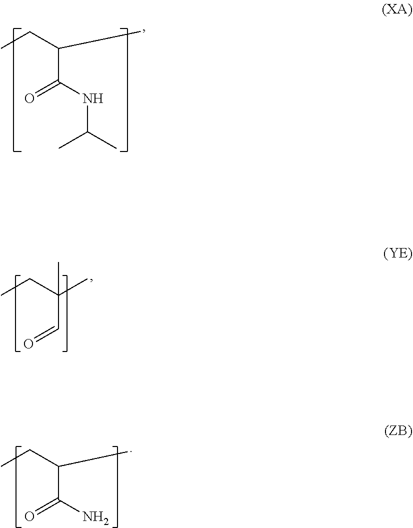

### BRIEF DESCRIPTION OF THE FIGURES

FIG. 1 is a pair of TEM images showing the nanoscale morphology of lauric acid-stabilised iron oxide nanoparticles. The atomic spacing of iron in the crystal is clearly visible.

FIG. 2 shows samples of (right) the phase-change polymer PNIPAM and (left) the complex PN-ION at a concentration of 30 mg/ml in water, below LCST (top) and above LSCT (bottom).

FIG. 3 is a UV-VIS graph of the absorbance at 800 nm of the complex formed in example 8 with respect to temperature.

FIG. 4 shows a solution comprising a complex containing a polymer bound to an iron oxide nanoparticle below the LCST (top) and above the LCST (bottom). A magnet is placed to the right of each image, left at t=0 s, right at t=30 s (i.e. the images on the right are images taken after 30 seconds in the presence of the magnet).

FIG. 5 is Disc centrifuge data for the ion complexes produced in example 9, showing a, peak intensity at a hydrodynamic diameter of 450 nm.

FIG. 6 is a pair of SEM images of the ion complexes produced in example 9, sputter coated in a gold-palladium alloy. The image on the top is of the complexes in powder form, while the image below is of complexes when dispersed by solvent evaporation.

FIG. 7 shows the inductive heating setup used in Example 10, involving a vial placed inside coil within insulation material, supported by a wooden clamp.

FIG. 8 shows the temperature of a solution comprising the complexes produced in example 8 as a function of time at different applied voltages in the inductive heating setup of FIG. 7.

FIG. 9 shows the amount of BSA released from immobilisation on the polymer polyNIPAM-co-acrolein over several cycles. Each cycle represents the solution being raised above the LCST of the polymer and then cooled down once more, before being precipitated out and a sample taken.

FIG. 10 is an HPLC chromatogram demonstrating the release of paclitaxel from BSA. The paclitaxel peak is present at 4.14, a known concentration of 600 uM (top) and a signal from a 0.1 mg/ml BSA-PAC complex. BSA degradation products are present from 1.1-1.65 minutes and the paclitaxel peak can be observed at 4.14 minutes.

FIG. 11 is a calibration curve for vinblastine immobilised on BSA.

FIGS. 12A and 12B indicate the effect on cell viability of complexes carrying paclitaxel were delivered to RD cell lines. FIG. 12A shows the outcome where the nanoparticle-polymer complexes were not activated by radio-waves, while FIG. 12B shows the outcome where the nanoparticle-polymer complexes were activated.

FIGS. 13A and 13B indicate the effect on cell viability of complexes carrying paclitaxel delivered to RH30 (Rhabdomyosarcoma) cells. FIG. 13A shows the outcome where the nanoparticle-polymer complexes were not activated by radio-waves, while FIG. 13B shows the outcome where the nanoparticle-polymer complexes were activated.

FIGS. 14A and 14B indicate the effect on cell viability of complexes carrying vinblastine on U87 cells. FIG. 14A shows the outcome where the nanoparticle-polymer complexes were not activated by radio-waves, while FIG. 14B shows the outcome where the nanoparticle-polymer complexes were activated.

FIG. 15 is a graph indicating the variation in the size of nanoparticle-polymer complexes according to the invention with temperature. As the temperature exceeds 40° C., the complexes reduce in size due to the phase change of the thermo-responsive polymer.

### DETAILED DESCRIPTION OF THE INVENTION

Unless defined otherwise, all technical and scientific terms used herein have the same meanings as commonly understood by one of skill in the relevant art. Publications cited herein and the materials for which they are cited are specifically incorporated by reference.

It should be appreciated that embodiments of the disclosure can be specifically combined together unless the context indicates otherwise. In particular, the following description concerns: a nanoparticle-polymer complex; a composition comprising the nanoparticle-polymer complex; an article comprising the nanoparticle-polymer complex; methods of using the nanoparticle-polymer complex; a composition for use in methods of therapy comprising the nanoparticle-polymer complex; and uses of the nanoparticle-polymer complex. Except where otherwise stated, discussion of an aspect of each embodiment of the invention also relates to that aspect in any other embodiment of the invention. For instance, discussion hereafter of “the polymer” or “the iron oxide nanoparticle” concerns not only the nanoparticle-polymer complex per se but also the polymer and iron oxide nanoparticle employed in the compositions, methods and uses of the invention.

The present invention will be described with respect to particular embodiments and drawings. However, the invention is not limited thereto but only by the claims. Those skilled in the art will recognize, or be able to ascertain using no more than routine experimentation, many equivalents to the specific embodiments of the invention described herein. Such equivalents are intended to be encompassed by the following claims.

### Definitions

As used in this specification and the appended claims, the singular forms “a”, “an”, and “the” include plural referents unless the content clearly dictates otherwise.

Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

“Alkyl” as used herein refers to monovalent straight-chained and branched alkyl groups. Typically, the alkyl group is a straight-chained alkyl group. Typically, an alkyl group has from 1 to 6 carbon atoms and so is a C₁₋₆ alkyl group, for example a C₁₋₄ alkyl group. Examples of alkyl groups include methyl and ethyl groups, and straight-chained or branched propyl, butyl and pentyl groups. Particular alkyl groups include methyl, ethyl, n-propyl, isopropyl, n-butyl, sec-butyl, isobutyl, and tert-butyl groups.

“Alkylene” as used herein refers to a divalent saturated hydrocarbon moiety which may be straight-chained or branched. Typically, the alkylene group is a straight-chained alkylene group. An alkylene group typically has from 1 to 6 carbon atoms (i.e. is a C₁₋₆ alkylene group). However, preferred alkylene groups include C₁₋₄ alkylene groups. Examples of alkylene groups include methylene (—CH₂—) and ethylene (—CH₂CH₂—) groups; also —CH(CH₃)—, and —CH(CH₂CH₃)—.

“Alkenyl” as used herein refers to a monovalent hydrocarbon moiety comprising one or more carbon-carbon double bonds. Typically an alkenyl group contains one carbon-carbon double bond. The hydrocarbon moiety may be a straight-chain or branched; typically, the hydrocarbon moiety is a straight chain. Typically, an alkenyl group has from 2 to 6 carbon atoms and so is a C₂₋₆ alkenyl group, for example a C₂₋₄ alkenyl group.

“Alkenylene” as used herein refers to a divalent hydrocarbon moiety comprising one or more carbon-carbon double bonds. Typically an alkenylene group contains one carbon-carbon double bond. The hydrocarbon moiety may be a straight-chain or branched; typically, the hydrocarbon moiety is a straight chain. An alkenylene group typically has from 2 to 6 carbon atoms (i.e. is a C₂₋₆ alkenylene group). Preferred alkenylene groups include C₂₋₄ alkenylene groups.

“Cycloalkyl” as used herein refers to monovalent groups derived from a saturated monocyclic hydrocarbon. A cycloalkyl group may typically have, for instance, 3 to 6 carbon atoms. Exemplary cycloalkyl groups include cyclopentyl and cyclohexyl groups.

“Cycloalkylene” as used herein refers to a divalent group derived from a saturated monocyclic hydrocarbon. A cycloalkylene may typically have 3 to 6 carbon atoms.

“Heterocycloalkyl” as used herein refers to monovalent saturated rings comprising at least one heteroatom selected from oxygen, sulphur and nitrogen, preferably oxygen or nitrogen. Heterocycloalkyl groups typically comprise one, two or three heteroatoms each independently selected from oxygen, sulphur and nitrogen, usually one such heteroatom. Heterocycloalkyl rings may be monocyclic (e.g. piperidinyl) or polycyclic (e.g. decahydroquinoline); preferably they are monocylic. A heterocycloalkyl group typically comprises from 5 to 14 carbon atoms. Exemplary heterocycloalkyl groups include piperidinyl and tetrahydropyranyl.

“Heterocycloalkylene” as used herein refers to divalent saturated rings comprising at least one heteroatom selected from oxygen, sulphur and nitrogen, preferably oxygen or nitrogen. Heterocycloalkylene groups typically comprise one, two or three such heteroatoms; usually one such heteroatom. Heterocycloalkylene rings may be monocyclic or polycyclic; preferably they are monocyclic. A heterocycloalkylene group typically comprises from 5 to 4 carbon atoms.

“Aryl” as used herein refers to a monovalent unsaturated aromatic carbocyclic group which may be monocyclic (for instance a phenyl group) or polycyclic, having multiple condensed rings (e.g. a naphthyl group). Preferably an aryl group is monocyclic. An aryl group typically contains from 6 to 14 carbon atoms. A preferred aryl group is phenyl.

“Arylene” as used herein refers to a divalent unsaturated aromatic carbocyclic group which may be monocyclic or polycyclic. Preferably an arylene group is monocyclic. An arylene group typically contains from 6 to 14 carbon atoms. A preferred arylene group is —C₆H₄—.

“Heteroaryl” as used herein refers to monovalent aromatic cyclic groups having at least one heteroatom selected from oxygen, sulphur and nitrogen. Heteroaryl groups typically comprise one, two or three heteroatoms each independently selected from oxygen, sulphur and nitrogen, usually one such heteroatom. Heteroaryl groups may be monocyclic or polycyclic; preferably they are monocyclic. A heteroaryl group typically comprises from 5 to 14 carbon atoms. Exemplary heteroaryl groups include pyridinyl and pyrimidinyl groups.

“Heteroarylene” as used herein refers to divalent aromatic cyclic groups having at least one heteroatom selected from oxygen, sulphur and nitrogen. Heteroarylene groups typically comprise one, two or three heteroatoms each independently selected from oxygen, sulphur and nitrogen, usually one such heteroatom. Heteroarylene groups may be monocyclic or polycyclic; preferably they are monocyclic. A heteroarylene group typically comprises from 5 to 14 carbon atoms.

The term “oxo” indicates a=O group. The term “hydroxy” indicates an —OH group.

Reference herein to any group in its protonated form should be taken to encompass reference to any deprotonated form which may be produced in solution. For instance, reference to “hydroxy” or “—COOH” should be taken to encompass such groups when deprotonated to —O— or —COO— in solution.

The term “halogen” as used herein is intended to include fluorine, chlorine, bromine and iodine atoms, typically fluorine, chlorine or bromine.

### Nanoparticle-Polymer Complex

The invention provides a nanoparticle-polymer complex comprising an iron oxide nanoparticle bound to a polymer capable of undergoing a phase change at a predetermined temperature. The nanoparticle-polymer complex of the invention may be referred to herein as the “nanoparticle-polymer complex” or “the complex”.

The polymer is chemically bound to the iron oxide nanoparticle. Typically, the polymer is ionically bound to the iron oxide nanoparticle. Ionic binding can be achieved by ionic interactions between charged groups on the polymer and the iron oxide nanoparticle. For instance, polymer may comprise —O⁻ groups, —COO⁻ groups or —NH₃⁺ groups (most usually —COO⁻ groups) which can form ionic interactions with the iron oxide nanoparticle.

Alternatively or additionally, the polymer may be bound to the iron oxide nanoparticle by hydrogen bonding. The iron oxide nanoparticle typically comprises —OH or —O⁻ groups on its surface and these may form hydrogen bonds with —O⁻ groups, —COO⁻ groups, —OH groups, —COOH groups, —NH₂ groups or —NH₃⁺ groups present in the polymer.

Thus, in a preferred aspect, the polymer comprises —OH, —COOH or —COO⁻ groups which are chemically bound to the iron oxide nanoparticle. These groups may be bound to the iron oxide nanoparticle by hydrogen bonding and/or ionic interactions. For example, the polymer may comprise carboxylate (—COO⁻) groups which are chemically bound to the iron oxide nanoparticle.

The groups which chemically bind to the iron oxide nanoparticle may form part of an anchoring group in the polymer, discussed in more detail below.

The polymer may be directly or indirectly bound to the iron oxide nanoparticle. Where the polymer is directly bound to the iron oxide nanoparticle, the polymer may comprise anchoring groups which directly interact with the iron oxide nanoparticle. Alternatively, where the polymer is indirectly bound to the iron oxide nanoparticle, the polymer may be bound to the iron oxide nanoparticle via another species. For instance, the polymer may be chemically bound (e.g. ionically bound or bound by hydrogen bonds) to a ligand which is attached to the iron oxide nanoparticle. Preferably, the polymer is directly bound to the iron oxide nanoparticle.

The complex typically has a core-shell type structure, wherein the iron oxide nanoparticle core is surrounded by the polymer. The surface of the iron oxide nanoparticle is therefore substantially or wholly covered by the polymer. In some embodiments, however, the polymer may cover only part of the surface of the iron oxide nanoparticle.

The complex typically has a largest dimension on the order of micrometres or nanometres. Accordingly, the complex may readily be administered to a patient. For instance, the complex typically has a maximum largest dimension of 50 μm or less, generally 10 μm or less. For instance, the largest dimension of the complex may be from 1 nm to 50 μm, preferably from 5 nm to 25 μm, more preferably from 0.1 μm to 10 μm.

The complex may comprise more than one iron oxide nanoparticle. For instance, the complex may comprise from one to ten iron oxide nanoparticles, e.g. 1, 2, 3, 4, or 5 iron oxide nanoparticles. Preferably, however, the complex contains only one iron oxide nanoparticle.

### Polymer

The complex comprises a polymer which is capable of undergoing a phase change at a predetermined temperature.

Polymers which are capable of undergoing a phase change dependent on temperature are known in the art. Such polymers are known as thermosensitive or thermo-responsive polymers. As explained in the background to the invention, in the presence of water such polymers exist in a solvated phase at a temperature below the LCST. However, on heating, such polymers undergo a phase transition at the LCST and adopt a globular conformation above the LCST. The globular conformation is insoluble or has a low solubility in water. Accordingly, on heating from below the LCST to above the LCST, such phase-change polymers will collapse and expel the aqueous solution which previously solvated the polymer.

The inventors have appreciated that this property of phase-change polymers may be used to release an active agent from a complex comprising an iron oxide nanoparticle via number of routes.

In the simplest example, the complex may initially comprise an active agent in a solution which solvates the polymer. Accordingly, when the iron oxide nanoparticle is heated and the phase change occurs, the active agent will be expelled from the complex together with the solvating solution when the polymer adopts its globular conformation. However, this method of release of an active agent has some drawbacks as the active agent may diffuse out of the complex before the complex is heated and the phase change occurs.

In another example, the complex may initially comprise an active agent bound to the iron oxide particle. The active agent may be bound to the iron oxide particle by ionic interactions and/or by hydrogen bonds. For instance, the active agent may form ionic bonds and/or hydrogen bonds with —OH or —O⁻ groups at the surface of the iron oxide nanoparticle. The active agent may alternatively be indirectly bound to the iron oxide nanoparticle, for instance the active agent may be covalently bound, ionically bound or bound by hydrogen bonds to a ligand attached to the surface of the iron oxide nanoparticle. As the polymer generally covers the entire surface of the iron oxide nanoparticle, in such embodiments, the active agent is typically trapped at the surface of the iron oxide nanoparticle. Thus, the active agent is not directly exposed to the environment. However, when the complex is heated and the polymer undergoes is phase transition and contracts, at least part of the surface of the iron oxide nanoparticle may become exposed to the environment. The active agent can then be released to the environment. For instance, where the active agent is bound to the iron oxide nanoparticle and the polymer present in the complex undergoes a phase change to the globular conformation, a solution surrounding the complex may be able to penetrate to the surface of the iron oxide nanoparticle and the active agent is released from the complex into the solution.

In yet another example, the nanoparticle-polymer complex may comprise an active agent which is bound to the polymer. The active agent may be bound to the polymer directly or via a carrier, as will be discussed in more detail below. This embodiment is advantageous as the binding of the active agent to the polymer stabilises the active agent and reduces the likelihood that it will be released before the iron oxide nanoparticle is heated by an alternating magnetic field. The inventors have surprisingly found that, even though the active agent is bound (directly or indirectly) to the polymer, initiating the phase change of the polymer will nonetheless cause the active agent to be released. This process may be driven by (i) the thermodynamic preference of the active agent (and any carrier, if present) to be solvated; and (ii) the thermodynamic preference of the thermo-responsive polymer in the globular phase to adopt a compact structure which does not have room for the active agent.

The active agent may be released from the iron oxide nanoparticle upon heating of the complex by any combination of these routes.

Examples of widely-used thermo-sensitive polymers are poly(N-isopropylacrylamide), PNIPAM; poly(N-vinyl caprolactam); and poly(vinyl methyl ether). These thermo-responsive polymers undergo a phase transition at a particular temperature (the characteristic LCST of the complex). However, these polymers are not directly suitable for use in a nanoparticle-polymer complex as described herein. That is because their LCST is generally much lower than usual human body temperatures. Accordingly, such polymers would immediately undergo a phase transition when brought into contact with a patient's body, or an in vitro sample at body temperature. Accordingly, these polymers would release a bound agent immediately; release of an active agent held in the complex could not be delayed until activation by an alternating magnetic field.

Further, the inventors have found that when thermo-responsible polymers are attached to an iron oxide nanoparticle, the observed LCST for the polymer in the complex tends to be lower than the LCST for the bulk polymer in isolation.

Accordingly, the complex of the invention comprises a polymer which is capable of undergoing a phase transition at a pre-determined temperature. By this is meant that the polymer is modified such that its LCST is adjusted to be suitable for the intended use of the nanoparticle-polymer complex.

For instance, where the complex is intended to be used in the human or animal body, it is important for the polymer to be adapted such that it undergoes a phase transition above the temperature of the body. Otherwise, the polymer will undergo its phase transition immediately when inserted into the body, and will release the active agent held in the polymer. This gives no opportunity for the complex to be transported to a particular desired location within the body before the active agent is released.

The average temperature of the human body is 37° C. Accordingly, typically the pre-determined temperature at which the polymer is capable of undergoing a phase change (that is, its LCST) is about 37° C. or more. For instance, the pre-determined temperature may be greater than 37° C. Preferably, however, the pre-determined temperature may be higher than this to ensure that local variations in body temperature do not cause release of the active agent and that the active agent will be released specifically when heated (typically by exposure to an alternating magnetic field). Accordingly, in a preferred embodiment, the pre-determined temperature is about 39° C. or more. For instance, the pre-determined temperature may be greater than 39° C. For instance, the pre-determined temperature may be about 39° C., or about 40° C., or about 41° C.

In some embodiments, the heating of the iron oxide nanoparticles may not be intended to substantially heat the surrounding environment. For instance, for in vivo applications of the complex, it may be desirable not to heat the tissue surrounding the complex in order to avoid damaging said tissue. Accordingly, the pre-determined temperature may be a temperature not too far above human body temperature to ensure that the temperature needed to release an active agent from the complex will not cause tissue damage. Thus, the pre-determined temperature may be lower than about 42° C., for instance the pre-determined temperature may be 42° C. or less. In a preferred embodiment, the pre-determined temperature may be from about 37° C. to about 44° C.; preferably from about 39° C. to about 42° C.

It will be appreciated that in other sensitive environments it may also be desirable to avoid local heating of the environment.

However, in some embodiments, it may be desired to use the complex to cause local heating of the environment as well as to release an active agent. In such cases, the pre-determined temperature may be rather higher. For instance, if the complex is intended to be used to release active agent in a tumour or tumour bed then it may be desirable to release the active agent and simultaneously perform a hyperthermic treatment. Thus, the pre-determined temperature may be up to about 50° C. for example up to 45° C. Accordingly, in another preferred embodiment, the pre-determined temperature may be from about 37° C. to about 50° C.; preferably from about 39° C. to about 48° C. or from about 39° C. to about 45° C.

Especially, the pre-determined temperature is greater than 40° C., for instance from 40° C. to 43° C., preferably from 40.5° C. to 42.5° C. or from 41° C. to 42° C., particularly about 41° C.

The phase change which the polymer undergoes in solution at the pre-determined temperature (the LCST) is reversible. Thus, on heating from below the pre-determined temperature to above the pre-determined temperature, the polymer will undergo a phase transition from a solvated phase to a globular conformation. However, on cooling from above the pre-determined temperature to below the pre-determined temperature, the polymer will return to its solvated phase.

This is useful as it allows the nanoparticle-polymer complex to be used repeatedly to release an active agent. When the complex is heated by exposure to an alternating magnetic field, the polymer will undergo a phase transition at the pre-determined temperature and release active agent. Once the alternating magnetic field is removed, the complex will cool. As the phase transition is reversible, the polymer will revert to its solvated state below the pre-determined temperature. The process may then be repeated, allowing the complex to be used for repeat release of the active agent at separated time points. The process is analogous to repeatedly squeezing a sponge.

In order to provide a polymer having the desired phase transition at the pre-determined temperature, the polymer may have a structure comprising one or more of a phase-change repeating unit; a hydrophilic repeating unit; and a binding repeating unit. Generally, the polymer comprises a phase-change repeating unit and a hydrophilic repeating unit. Preferably, the polymer also comprises a binding repeating unit. The structure of the polymer is described in more detail below.

The polymer typically comprises a phase-change repeating unit. A “phase-change repeating unit” is a thermosensitive moiety. The phase-change unit has a high affinity for water at low temperatures and a low affinity for water at higher temperatures. Typically, the phase-change unit is soluble in aqueous solution below the pre-determined temperature and is not soluble or is sparingly soluble above the pre-determined temperature.

Where the polymer comprises a phase-change repeating unit, the LCST of the polymer differs from the LCST of a polymer consisting entirely of the phase-change repeating unit. That is, the pre-determined temperature of the polymer differs from the LCST of the phase-change repeating unit. Generally, the pre-determined temperature is greater than the LCST of the thermo-sensitive polymer on which it is based. Accordingly, the pre-determined temperature is generally greater than the LCST of a polymer consisting entirely of the phase-change repeating unit.

The phase-change unit is a repeating unit of the polymer.

The phase change unit may have formula (X). Accordingly, the polymer may comprise a repeating unit of formula (X):

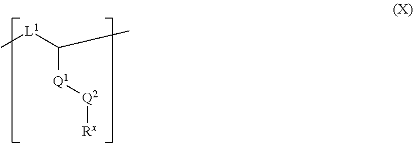

L¹ is absent or may be selected from C₁₋₆ alkylene, C₂₋₆ alkenylene, C₃₋₆ cycloalkylene, arylene, heteroarylene, heterocycloalkylene, C₁₋₆ alkylene-arylene, and C₁₋₆ alkylene-arylene-C₁₋₆ alkylene; preferably L¹ is absent or is C₁₋₆ alkylene; for instance L¹ may be absent or may be —CH₂—, —CH₂CH₂— or —CH₂CH₂CH₂—. Most preferably L¹ is —CH₂—.
Q¹ is —O—, —NR^{x}— or —C(═O)—.
Q² is absent or is —O—, —NR^{x}— or —C(═O)—.

The or each R^{x} is independently selected from C₁₋₆ alkyl, or two R^{x} groups may be joined together to form a 3- to 8-membered carbocyclic or heterocyclic ring including Q¹ and/or (where present) Q²; preferably the or each R^{x} is independently selected from C₁₋₆ alkyl, or two R^{x} groups may be joined together to form a 5- to 7-membered heterocyclic ring including Q¹ and/or (where present) Q².

Each position on (X) capable of substitution may be optionally substituted. For instance (X) may be substituted with one or more substituents each independently selected from hydroxy, C₁₋₆ alkyl, C₂₋₆ alkenyl or halogen, preferably selected from C₁₋₃ alkyl. Where the phase change unit (X) is substituted, it is usually substituted by 1, 2 or 3 substituents or; more preferably the phase change unit (X) is unsubstituted. An example of a substituted phase change unit (X) is a unit of formula (X′) as follows:

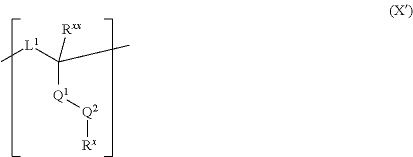

wherein L¹, Q¹, Q² and R^{x} are as defined above and R^{xx} is a substituent selected from hydroxy, C₁₋₆ alkyl, C₂₋₆ alkenyl and halogen, and is preferably C₁₋₃ alkyl, for instance methyl.

The phase change unit of formula (X) may be a unit of formula (XA):

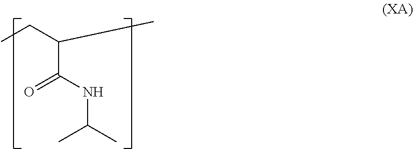

which may be optionally substituted as described above. Thus, each position on (XA) capable of substitution may be optionally substituted, as described above for formula (X). An example of a substituted phase change unit (XA) is a unit of formula (XA′) as follows:

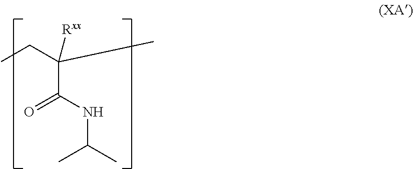

wherein R^{xx} is a substituent selected from hydroxy, C₁₋₆ alkyl, C₂₋₆ alkenyl and halogen, and is preferably C₁₋₃ alkyl. R^{xx} may for instance be methyl; thus, the polymer may comprise a repeating unit derived from N-isopropylmethacrylamide.

Preferably, the phase change unit of formula (XA) is unsubstituted.

Accordingly, in a preferred embodiment, the polymer comprises a repeating unit derived from N-isopropylacrylamide. By “derived from” is meant that the unit is the unit obtained when the monomer N-isopropylacrylamide is polymerised. Thus, in some instances the polymer comprises poly(N-isopropylacrylamide).

Alternatively, the phase change unit of formula (X) may be a unit of formula (XB):

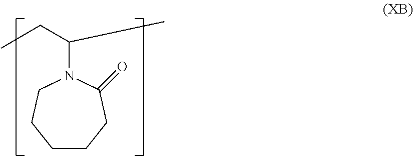

which may be optionally substituted as described above. Preferably, the phase change unit of formula (XB) is unsubstituted. Accordingly, in a preferred embodiment, the polymer comprises a repeating unit derived from N-vinyl caprolactam. By “derived from” is meant that the unit is the unit obtained when the monomer N-vinyl caprolactam is polymerised. Thus, in some instances the polymer comprises poly(N-caprolactam).

Alternatively, the phase change unit of formula (X) may be a unit of formula (XC):

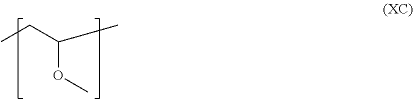

which may be optionally substituted as described above. Preferably, the phase change unit of formula (XC) is unsubstituted. Accordingly, in a preferred embodiment, the polymer comprises a repeating unit derived from vinyl methyl ether. By “derived from” is meant that the unit is the unit obtained when the monomer methyl vinyl ether is polymerised. Thus, in some instances, the polymer comprises poly(methyl vinyl ether).

The polymer may comprise one or more types of phase change repeating unit. For instance, the polymer may comprise repeating units of formula (XA) and/or (XB) and/or (XC). Typically, polymer comprises one or two types of phase change unit and generally comprises one kind of phase change unit. Preferably, the polymer comprises a phase change unit of formula (XA) or (XB) or (XC); most preferably the polymer comprises a phase change unit of formula (XA).

In order to drive the phase change at the pre-determined temperature, the polymer must contain a substantial amount of the phase change repeating unit. The polymer generally contains at least 25% by weight of the phase change unit(s), with respect to the total weight of the polymer. Generally, the polymer comprises at least 30% by weight of the phase change unit(s). For instance, the polymer may comprise from about 25 wt % to about 90 wt %; or about 30 wt % to about 60 wt %; or about 35 wt % to about 50 wt % or about 35 to 40% by weight of the phase-change repeating unit(s). For instance, the polymer may contain from about 25 wt % to about 90 wt %; or about 30 wt % to about 60 wt %; or about 35 wt % to about 50 wt % or about 35 wt % to about 40 wt % of a repeating unit derived from N-isopropylacrylamide. These values can provide an LCST which is quite different to that of a polymer consisting only of the phase-change repeating unit. For instance, where the repeating unit is N-isopropylacrylamide, the above values may provide a polymer with an LCST differing from that of pure pNIPAM, preferably of around 41° C. or even higher.

In other examples, the polymer may contain a greater amount of the phase-change unit. This may be useful where the desired LCST of the polymer is closer to that of a polymer consisting entirely of the phase-change repeating unit. For instance, the polymer may contain at least 40% by weight of the phase-change repeating unit. E.g. the polymer may contain from about 40 wt % to about 99 wt %; or about 50 wt % to about 95 wt %; or about, or about 55 wt % to about 90 wt %; or about 60 wt % to about 85 wt % by weight of the phase-change repeating unit(s). For instance, the polymer may contain from about 40 wt % to about 99 wt %; or about 50 wt % to about 95 wt %; or about, or about 55 wt % to about 90 wt %; or about 60 wt % to about 85 wt % by weight of a repeating unit derived from N-isopropylacrylamide.

The phase-change unit is incorporated into the polymer by including a “phase-change monomer” in a polymerisation process to produce the polymer. Suitable examples of phase-change monomers include N-isopropylacrylamide; N-vinyl caprolactam; and methyl vinyl ether. The skilled person will readily appreciate the monomers that would be used to incorporate a phase-change repeating unit of formula (X) above.

The polymer is typically capable of binding to an active agent (where present) or to a carrier (where present). In order to provide this functionality, a “binding unit” may be incorporated into the polymer. The binding unit is a repeating unit which is capable of forming a chemical bond to a carrier or an active agent. Typically, the binding unit is capable of forming a covalent bond to a carrier or an active agent; preferably a reversible covalent bond. A “reversible” covalent bond is a covalent bond which can be broken with relative ease, for instance under physiological conditions. Examples of reversible covalent bonds include sulphur bridges (S—S bonds) and C═N bonds.

The binding unit need not necessarily be present, for instance, where it is not intended to bind an active agent or carrier to the polymer (as the active agent may be incorporated by other means such as binding to the iron oxide nanoparticle). However, it is preferred that the binding unit is present.

The binding unit is a repeating unit as it is repeated throughout the polymer.

Thus, the binding unit may comprise a nucleophilic group. Accordingly, the binding unit may be referred to as a nucleophilic repeating unit. For instance, the binding unit may comprise one or more of an oxo group, an=NH group, an=S group, a hydroxy group, an amino (—NH₂) group or a thiol (—SH) group. Preferably, the binding unit may comprise an oxo group, a C═S bond, an —NH₂ group, a hydroxy group or a thiol group. More preferably the binding unit may comprise an oxo group or a thiol (—SH) group. Further preferably, the binding unit comprises an aldehyde group (—C(═O)H) or a thiol (—SH) group. Most preferably, the binding unit comprises an aldehyde group (—C(═O)H).

Accordingly, the polymer may comprise a repeating unit of formula (Y):

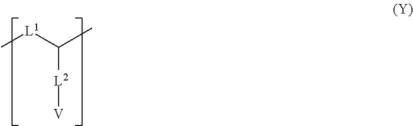

L¹ and L² are linkers or absent; and V is a nucleophilic group.
L¹ is absent or may be selected from C₁₋₆ alkylene, C₂₋₆ alkenylene, C₃₋₆ cycloalkylene, arylene, heteroarylene, heterocycloalkylene, C₁₋₆ alkylene-arylene, and C₁₋₆ alkylene-arylene-C₁₋₆ alkylene; preferably L¹ is absent or is C₁₋₆ alkylene; for instance L¹ may be absent or may be —CH₂—, —CH₂(CH₃)—, —CH₂(CH₂CH₃)—, —CH₂CH₂— or —CH₂CH₂CH₂—. Preferably L¹ is —CH₂—, —CH₂(CH₃)—, —CH₂(CH₂CH₃)—; most preferably L¹ is —CH₂—.
L² is absent or may be selected from C₁₋₆ alkylene, C₂₋₆ alkenylene, C₃₋₆ cycloalkylene, arylene, heteroarylene, heterocycloalkylene, C₁₋₆ alkylene-arylene, and C₁₋₆ alkylene-arylene-C₁₋₆ alkylene; preferably L² is absent or is C₁₋₆ alkylene; for instance L² may be absent or may be —CH₂—, —CH₂CH₂— or —CH₂CH₂CH₂—. More preferably L² is absent or is —CH—; most preferably, L² is absent.

V is a nucleophilic moiety. Preferably V is selected from —C(═X)—R, and —XR where X is O, S or NR. More preferably V is selected from —C(═S)R, —C(═NR)R, —C(═O)R, —SR, —OR, and —NR₂; more preferably V is selected from —C(═O)R, —SR, and —OR; more preferably from —C(═O)R and —SR. Most preferably V is —C(═O)R.

Where present, each R is independently selected from H or C₁₋₆ alkyl, more preferably H or C₁₋₃ alkyl, most preferably H.

Each position on (Y) capable of substitution may be optionally substituted. For instance (Y) may be substituted with one or more substituents each independently selected from hydroxy, C₁₋₆ alkyl, C₂₋₆ alkenyl, C₃₋₆ cycloalkyl, aryl, heteroaryl, heterocycloalkyl, C₁₋₆ alkylene-aryl, arylene-C₁₋₆ alkyl, —NR₂, —COR, —COOR or halogen. Preferably each substituent is independently selected from hydroxy, C₁₋₆ alkyl, C₂₋₆ alkenyl or halogen, more preferably C₁₋₃ alkyl, most preferably methyl. Where the binding unit (Y) is substituted, it is usually substituted by 1, 2 or 3 substituents; preferably by one substituent; particularly preferably by one methyl group.

In a particularly preferred embodiment, the carbon atom bound to both L¹ and L² (or, where L² is absent, V) is substituted by one substituent. The substituent is preferably selected from C₁₋₃ alkyl, most preferably methyl.

In another preferred embodiment, the binding unit (Y) is unsubstituted.

For instance, the binding unit (Y) may be a binding unit of formula (YA):

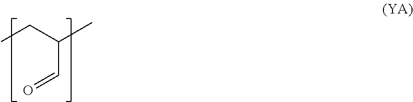

which may be optionally substituted as described in connection with (Y). Preferably, the binding unit of formula (YA) is unsubstituted. Accordingly, in a preferred embodiment, the polymer comprises a repeating unit derived from acrolein.

In another example, the binding unit (Y) may be a binding unit of formula (YB):

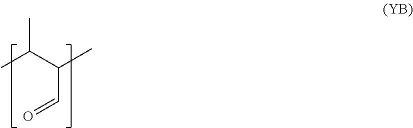

which may be optionally substituted as described in connection with (Y). Preferably, the binding unit of formula (YB) is unsubstituted. Accordingly, in a preferred embodiment, the polymer comprises a repeating unit derived from crotonaldehyde.

In another example, the binding unit (Y) may be a binding unit of formula (YC):

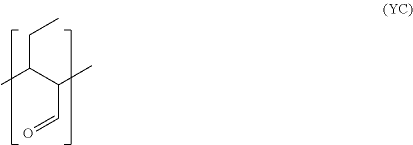

which may be optionally substituted as described in connection with (Y). Preferably, the binding unit of formula (YC) is unsubstituted. Accordingly, in a preferred embodiment, the polymer comprises a repeating unit derived from 2-pentenal, e.g. trans-2-pentenal.

In another example, the binding unit (Y) may be a binding unit of formula (YD):

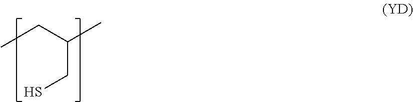

which may be optionally substituted as described in connection with (Y). Preferably, the binding unit of formula (YD) is unsubstituted. Accordingly, in a preferred embodiment, the polymer comprises a repeating unit derived from allyl mercaptan, also referred to as allyl thiol.

In another example, the binding unit (Y) may be a binding unit of formula (YE):

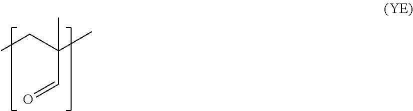

which may be optionally substituted as described in connection with (Y). Preferably, the binding unit of formula (YE) is unsubstituted. Accordingly, in a preferred embodiment, the polymer comprises a repeating unit derived from methacrolein.

The polymer may comprise one or more types of binding unit. For instance, the polymer may comprise repeating units of formula (YA) and/or (YB) and/or (YC) and/or (YD) and/or (YE). It may be desirable to include more than one type of binding unit where more than one type of active agent or carrier is to be attached to the nanoparticle-polymer complex. Typically, polymer comprises one or two types of binding unit and generally comprises one kind of binding unit. Preferably, the polymer comprises a binding unit of formula (YA) or (YB) or (YC) or (YD) or (YE); more preferably the polymer comprises a binding unit of formula (YA) or (YD) or (YE); most preferably the polymer comprises a binding unit of formula (YE).

Where a binding unit is present, the polymer comprises enough of the binding unit to provide sufficient loading of the active agent (and optionally the carrier) to the binding unit. The amount of the binding unit can therefore be low or can be high. Accordingly, where the polymer comprises a binding unit, it generally comprises at least 0.1 wt % of the binding unit compared to the total weight of the polymer. For instance, the polymer may comprise at least 0.5 wt % or at least 1 wt % of the binding unit. Typically, the polymer comprises from about 1 wt % to about 40 wt %; preferably about 5 wt % to 35 wt %, for example about 10 wt % to 30 wt % preferably 15 wt % or 25 wt % of the binding unit. For instance, the polymer may comprise about 20 wt % of the binding unit.

The binding unit is incorporated into the polymer by including a “binding monomer” in a polymerisation process to produce the polymer. The binding monomer may be selected for its ability to bind to an active agent or a carrier. Suitable examples of a binding monomer suitable to produce the binding units of formula (Y) above.

For instance, the binding monomer may be a monomer comprising a C═C double bond and a nucleophilic group such as a thiol group or a carbonyl group. Examples of suitable binding monomers include methacrolein, allyl mercaptan, acrolein, crotonaldehyde and 2-pentenal (particularly trans-2-pentenal). Preferred examples are methacrolein, acrolein and allyl mercaptan, particularly methacrolein or acrolein, most preferably methacrolein.

The polymer typically comprises a LCST-adjusting unit. The type and amount of the LCST-adjusting unit is adjusted in order to ensure that the LCST of the polymer is the desired pre-determined temperature.

The LCST-adjusting unit is a repeating unit as it is repeated throughout the polymer.

Typically the LCST-adjusting unit is a hydrophilic unit, meaning that it comprises a hydrophilic moiety. Inclusion of a hydrophilic moiety together with a phase-change repeating unit as described above increases the affinity of the polymer for water and raises its LCST. Accordingly, the LCST-adjusting unit is a hydrophilic unit where the pre-determined temperature is higher than the LCST of a polymer comprising only the phase change repeating unit.

Usually, the LCST-adjusting unit comprises a carbonyl moiety. For instance, the LCST-adjusting unit may comprise a carboxylic acid group —COOH, an ester group —COOR or an amide group —CONR².

The polymer may comprise an LCST-adjusting unit of formula (Z):

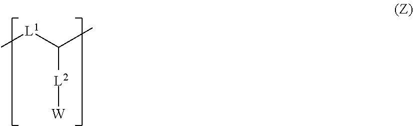

L¹ and L² are linkers or absent; and W is a hydrophilic group.
L¹ is absent or may be selected from C₁₋₆ alkylene, C₂₋₆ alkenylene, C₃₋₆ cycloalkylene, arylene, heteroarylene, heterocycloalkylene, C₁₋₆ alkylene-arylene, and C₁₋₆ alkylene-arylene-C₁₋₆ alkylene; preferably L¹ is absent or is C₁₋₆ alkylene; for instance L¹ may be absent or may be —CH₂—, —CH₂(CH₃)—, —CH₂(CH₂CH₃)—, —CH₂CH₂— or —CH₂CH₂CH₂—. Preferably L¹ is —CH₂—, —CH₂(CH₃)—, —CH₂(CH₂CH₃)—; most preferably L¹ is —CH₂—.
L² is absent or may be selected from C₁₋₆ alkylene, C₂₋₆ alkenylene, C₃₋₆ cycloalkylene, arylene, heteroarylene, heterocycloalkylene, C₁₋₆ alkylene-arylene, and C₁₋₆ alkylene-arylene-C₁₋₆ alkylene; preferably L² is absent or is C₁₋₆ alkylene; for instance L² may be absent or may be —CH₂—, —CH₂CH₂— or —CH₂CH₂CH₂—. Most preferably L² is absent.
W is a hydrophilic moiety. Preferably W is selected from —C(═X)—X—R, where each X is independently selected from O, S or NR. More preferably W is selected from —C(═X)—X—R, where each X is independently selected from O or NR. Further preferably W is selected from —C(═O)OR and —C(═O)—NR₂. Most preferably W is —C(═O)—NR₂.

Where present, each R is independently selected from H or C₁₋₆ alkyl, more preferably H or C₁₋₃ alkyl, most preferably H.

Each position on (Z) capable of substitution may be optionally substituted. For instance (Z) may be substituted with one or more substituents each independently selected from hydroxy, C₁₋₆ alkyl, C₂₋₆ alkenyl, C₃₋₆ cycloalkyl, aryl, heteroaryl, heterocycloalkyl, C₁₋₆ alkylene-aryl, arylene-C₁₋₆ alkyl, —NR₂, —COR, —COOR or halogen. Preferably each substituent is independently selected from hydroxy, C₁₋₆ alkyl, C₂₋₆ alkenyl or halogen, most preferably C₁₋₃ alkyl; more preferably (Z) is unsubstituted.

For instance, the LCST-adjusting unit of formula (Z) may be a unit of formula (ZA):

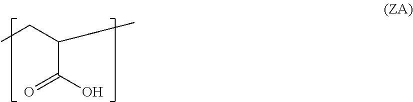

which may be optionally substituted as described in connection with (Z). An example of a substituted LCST-adjusting unit of formula (ZA) is a unit of formula (ZA′)

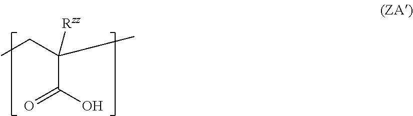

wherein R^{zz} is a substituent selected from any of the substituents listed above in connection with formula (Z). R^{zz} may for instance be C₁₋₆ alkyl, for instance C₁₋₃ alkyl. Preferably, R^{zz} is methyl. Thus, the polymer may comprise a repeating unit derived from methacrylic acid.

More preferably, the LCST-adjusting unit of formula (ZA) is unsubstituted. Accordingly, in a preferred embodiment, the polymer comprises a repeating unit derived from acrylic acid.

Alternatively, the LCST-adjusting unit of formula (Z) may be a unit of formula (ZB):

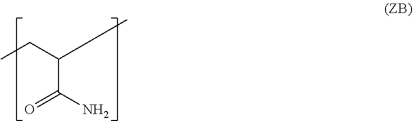

which may be optionally substituted as described in connection with (Z) or (ZA). Preferably, the LCST-adjusting unit of formula (ZB) is unsubstituted. Accordingly, in a preferred embodiment, the polymer comprises a repeating unit derived from acrylamide.

The polymer may comprise one or more types of LCST-adjusting repeating unit. For instance, the polymer may comprise repeating units of formula (ZA) and/or (ZB). Typically, the polymer comprises one or two types of LCST-adjusting unit and generally comprises one kind of LCST-adjusting unit. Preferably, the polymer comprises an LCST-adjusting unit of formula (ZA) or (ZB), particularly preferably (ZB).

The polymer comprises an amount of the LCST-adjusting repeating unit as needed to ensure that the LCST of the polymer is fixed at the pre-determined temperature. Accordingly, where the LCST of the polymer (for instance a polymer comprising a phase change repeating unit and a binding repeating unit) is close to or equal to the pre-determined temperature, little or no LCST-adjusting unit is needed. However, typically an LCST-adjusting unit is present, at least in a small amount. For instance, the polymer typically comprises at least 0.1 wt % of the LCST-adjusting unit, by total weight of the polymer and usually comprises at least 1 wt % of the LCST-adjusting unit. Typically, the polymer comprises from about 5 wt % to about 70 wt %, generally about 10 wt % to about 65 wt %, preferably about 20 wt % to about 60 wt %, more preferably about 40 wt % to about 55 wt % of the LCST-adjusting unit. For example, the polymer may comprise from about 45 wt % to about 50 wt % of the LCST-adjusting unit. Such values can provide a pre-determined LCST temperature for the polymer of 41° C. or more.

In other cases, the amount of LCST-adjusting unit may be less (for instance where a slightly lower LCST is desired). In such embodiments, typically, the polymer comprises about 1 wt % to about 50 wt %, generally about 5 wt % to about 45 wt %, for example about 10 wt % to about 40 wt % of the LCST-adjusting unit. By way of example, the polymer may comprise about 20 wt % or 30 wt % of the LCST-adjusting unit.

The LCST-adjusting unit is incorporated into the polymer by including an “LCST-adjusting monomer” in a polymerisation process to produce the polymer. The LCST-adjusting monomer is the monomer from which the LCST-adjusting repeating unit is derived. The LCST-adjusting monomer is typically a hydrophilic monomer, comprising a hydrophilic group. The skilled person will readily appreciate the monomers that would be used to incorporate a phase-change repeating unit of formula (Z) above.

The LCST-adjusting monomer will typically comprise a C═C double bond and one or more of a carboxylic acid group, an ester group, or an amide group. Suitable examples of the hydrophilic monomer are acrylic acid or acrylamide. Acrylamide is particularly preferred.

Some exemplary combinations of the phase change unit, the binding unit and the LCST-adjusting unit will now be described.

The first group of preferred embodiments concern exemplary polymers where the pre-determined temperature is preferably 39° C. or more. In all of these combinations it is preferred that the phase change unit comprises at least about 40% by weight of the polymer; the binding unit (where present) comprises at least 1% by weight of the polymer; and the LCST-adjusting unit comprises at least about 1 wt % of the polymer. More preferably, the phase change unit comprises about 40 wt % to 99 wt %; the binding unit (where present) comprises about 1 wt % to 40 wt %; and the LCST-adjusting unit comprises about 1 wt % to about 50 wt % of the polymer. Still more preferably, the phase change unit comprises about 50 wt % to 95 wt %; the binding unit (where present) comprises about 5 wt % to 35 wt %; and the LCST-adjusting unit comprises about 5 wt % to about 45 wt % of the polymer.
The polymer may comprise a phase-change repeating unit of formula (X) and an LCST-adjusting unit of formula (Z). Preferably, the polymer comprises a phase-change repeating unit of formula (X), a binding repeating unit of formula (Y) and an LCST-adjusting unit of formula (Z).
For instance, the polymer may comprise a phase change repeating unit of formula (XA), (XB) or (XC) and an LCST-adjusting repeating unit of formula (ZA) or (ZB). Preferably, the polymer comprises a phase change repeating unit of formula (XA), (XB) or (XC); a binding unit of formula (YA), (YB), (YC) or (YD) and an LCST-adjusting repeating unit of formula (ZA) or (ZB).
In a particularly preferred embodiment, the polymer may comprise a phase change repeating unit of formula (XA), a binding unit of formula (YA) or (YD) and an LCST-adjusting repeating unit of formula (ZA) or (ZB).
For example, the polymer may comprise a phase change repeating unit of formula (XA), a binding unit of formula (YA) and an LCST-adjusting unit of formula (ZA). Alternatively, the polymer may comprise a phase change repeating unit of formula (XA), a binding unit of formula (YD) and an LCST-adjusting unit of formula (ZB).

The second group of preferred embodiments concern exemplary polymers where the pre-determined temperature is preferably 40° C. or more, particularly preferably 41° C. or more. In all of these combinations it is preferred that the phase change unit comprises at least about 25% by weight of the polymer; the binding unit (where present) comprises at least 1% by weight of the polymer; and the LCST-adjusting unit comprises at least about 5 wt % of the polymer. More preferably, the phase change unit comprises about 25 wt % to 90 wt %; the binding unit comprises about 1 wt % to 40 wt %; and the LCST-adjusting unit comprises about 5 wt % to about 70 wt % of the polymer. Still more preferably, the phase change unit comprises about 30 wt % to 60 wt %; the binding unit comprises about 5 wt % to 35 wt %; and the LCST-adjusting unit comprises about 10 wt % to about 65 wt % of the polymer. Still more preferably, the phase change unit comprises about 35 to 50 wt %; the binding unit comprises about 10 to 30 wt %; and the LCST-adjusting unit comprises about 20 to 60 wt % of the polymer. Further preferably, the phase change unit comprises about 35 wt % to 45 wt %; the binding unit comprises about 15 wt % to 25 wt %; and the LCST-adjusting unit comprises about 40 wt % to about 55 wt % of the polymer. Most preferably, the phase change unit comprises about 35 to 40 wt %; the binding unit comprises about 15 to 20 wt %; and the LCST-adjusting unit comprises about 45 to 50 wt % of the polymer.
The polymer comprises a phase-change repeating unit of formula (X), a binding repeating unit of formula (Y) and an LCST-adjusting unit of formula (Z).
For instance, the polymer comprises a phase change repeating unit of formula (XA), (XB) or (XC); a binding unit of formula (YA), (YB), (YC), (YD) or (YE) and an LCST-adjusting repeating unit of formula (ZA) or (ZB).
In a particularly preferred embodiment, the polymer may comprise a phase change repeating unit of formula (XA), a binding unit of formula (YE) or (YD) and an LCST-adjusting repeating unit of formula (ZB).

In an example, the polymer may comprise a unit of formula (I)

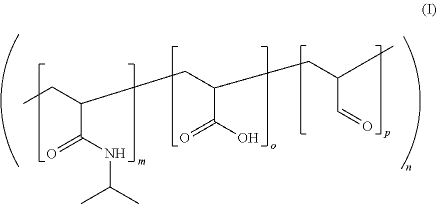

wherein n, m, o and p are integers. Typically n, m, o and p are not 0. For instance, each of n, m, o and p may independently be 1, 2, 3, 4, 5, 6, 7, 8, 9, or 10. The component repeating units may be in any order.

In another example, the polymer may comprise a unit of formula (II):

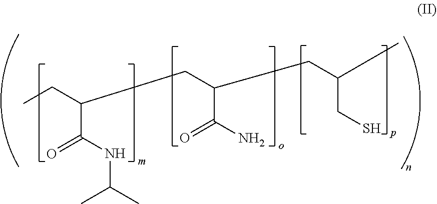

wherein n, m, o and p are integers. Typically n, m, o and p are not 0. For instance, each of n, m, o and p may independently be 1, 2, 3, 4, 5, 6, 7, 8, 9, or 10. The component repeating units may be in any order.

In another example, the polymer may comprise a unit of formula (III):

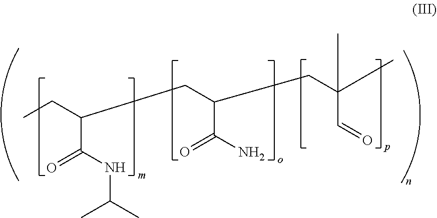

wherein n, m, o and p are integers. Typically n, m, o and p are not 0. For instance, each of n, m, o and p may independently be 1, 2, 3, 4, 5, 6, 7, 8, 9, or 10. The component repeating units may be in any order.

Above are described the features of the polymer which enable it to undergo a phase change at the pre-determined temperature and bind an active agent. Another important aspect of the polymer is that it is chemically bound to the iron oxide particle, directly or indirectly. Accordingly, the polymer typically comprises an “anchoring unit”. An anchoring unit is a group capable of chemically binding to the iron oxide nanoparticle (directly or indirectly). For instance, the anchoring unit may comprise a moiety which is capable of forming ionic bonds and/or hydrogen bonds. The anchoring unit typically comprises an ionic group, for instance a carboxylate group. Alternatively, the anchoring unit may comprise hydroxyl groups which can form H-bonds. An example of the anchoring unit is a unit derived from the monomer DDMAT: 2-(Dodecylthiocarbonothioylthio)-2-methylpropionic acid). DDMAT has the structure:

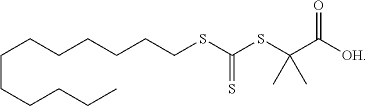 [SMILES: CCCCCCCCCCCCSC(=S)SC(C)(C)C(=O)O]

The anchoring unit is a repeating unit as it is repeated throughout the polymer.

The anchoring unit is incorporated into the polymer by including an “anchoring monomer” in a polymerisation process to produce the polymer. The anchoring monomer is the monomer from which the anchoring repeating unit is derived. The anchoring monomer will typically comprise a polymerisable moiety and an ionic or ionisable moiety. Examples of the polymerisable moiety include groups susceptible to RAFT polymerisation such as double bonds, for instance C═S or C═C double bonds; particularly a —C(═S)—S— moiety or a —S—C(═S)—S— moiety. Examples of ionic or ionisable moieties include carboxylate groups. A suitable example of the anchoring monomer is DDMAT.

In the nanoparticle-polymer complex, some or all of the anchoring groups are chemically bound to the iron oxide nanoparticle, for instance by hydrogen bonds or ionic bonds.

The polymer may of course comprise other types of repeating unit. For instance, the polymer may comprise repeating units derived from ethylene glycol to cap the polymer during polymerisation and to improve biocompatibility. Accordingly, the polymer may comprise polyethylene glycol.

The polymer may further comprise water, or an aqueous solution.

The nanoparticle-polymer complex may comprise one or more polymers as described above. There are various situations where it is useful to include a second or further polymer in the complex. Where a nanoparticle-polymer complex comprises two or more polymers as described above, the polymers may be referred to as “the first polymer”, “the second polymer” and so on. The nanoparticle-polymer complex may for instance comprise a first polymer associated with a first active agent (via a carrier or otherwise) and a second polymer associated with a second active agent (via a carrier or otherwise). The first polymer may be capable of undergoing a phase transition at a first pre-determined temperature and the second polymer may be capable of undergoing a phase transition at a second pre-determined temperature. The first pre-determined temperature may be different to the second pre-determined temperature. Accordingly, it is possible for the user to choose whether one or both of the first and second active agents are released. If the complex is heated to a temperature which is above both pre-determined temperatures, both the first and second active agents will be released. However, if the complex is heated to a temperature between the first pre-determined temperature and the second pre-determined temperature, only the polymer which has the lower pre-determined temperature will undergo a phase change causing release of the associated active agent.

In addition to a polymer as described above, the nanoparticle-polymer complex may comprise one or more additional polymers which are not as described above (i.e. a polymer which is not capable of undergoing a phase change at a predetermined temperature). Such a polymer may be referred to as a “supporting polymer”. An example of a supporting polymer is polyethylene glycol, which may be included in the complex to aid biocompatibility.

More typically the complex does not comprise a supporting polymer. Generally the complex contains a single polymer.

In an embodiment, the invention provides a polymer as described above. The polymer is provided independently of the nanoparticle-polymer complex. In particular, the invention provides a thermosensitive polymer comprising a phase change repeating unit of formula (XA), a binding repeating unit of formula (YE) and a hydrophilic repeating unit of formula (ZB):

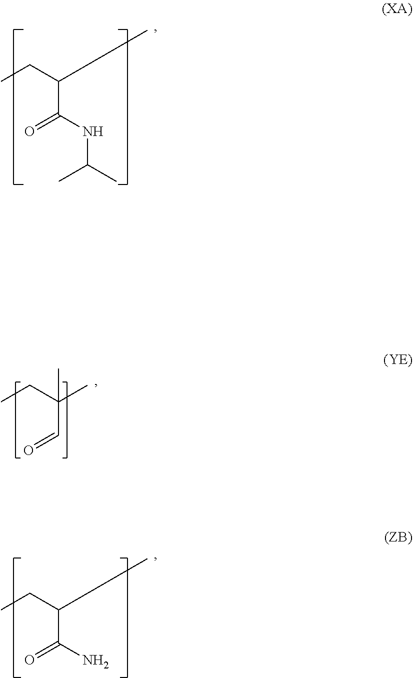

The repeating units (XA), (YE) and (ZB) may be present in any order and in varying amounts. In some embodiments, the thermosensitive polymer may comprise a unit of formula (III):

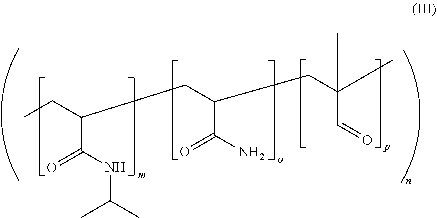

wherein n, m, o and p are integers.

The LCST of the thermosensitive polymer is generally greater than 37° C. (although can be tuned to a lower value if required for a particular use). Preferably, the LCST of the thermosensitive polymer is 39° C. or more. Particularly preferably, the thermosensitive polymer has an LCST of 41° C. or more. For example, the thermosensitive polymer may have an LCST of from 39° C. to 50° C.; preferably from 40° C. to 45° C.; more preferably from 41° C. to 43° C.; most preferably from 41° C. to 42° C. Generally, the LCST of the polymer is around 41° C., e.g. from 40.5° C. to 41.5° C.

The amounts of each of (XA), (YE) and (ZB) in the polymer may be varied. Generally, the thermosensitive comprises from 25 wt % to 90 wt % of (XA), 1 wt % to 40 wt % of (YE), and 5 wt % to 70 wt % of (ZB); preferably from 30 wt % to 60 wt % of (XA), from 5 wt % to 35 wt % of (YE) and from 10 wt % to about 65 wt % of (ZB); more preferably from 35 wt % to 50 wt % of (XA), from 10 wt % to 30 wt % of (YE), and from 20 wt % to 60 wt % of (ZB); still more preferably from 35 wt % to 45 wt % of (XA), from 15 wt % to 25 wt % of (YE) and from 40 wt % to 55 wt % of (ZB); most preferably from 35 wt % to 40 wt % of (XA), from 15 wt % to 20 wt % of (YE), and from 45 wt % to 50 wt % of (ZB).

The thermosensitive polymer may optionally comprise a carrier as described herein. The carrier is generally a protein, preferably selected from bovine serum albumin or human serum albumin. The carrier may be immobilised on the polymer, for instance reversibly covalently bound to the polymer. Preferably, the carrier is bound to the polymer via a —C═N— bond. Such a bond may form with an amino group of the carrier at the aldehyde position of the (YE) repeating unit.

The thermosensitive polymer may further comprise an active agent and/or a secondary active agent as described herein.

For instance, the thermosensitive polymer may comprise an active agent which is a chemotherapeutic agent. The active agent may be selected from one or more of paclitaxel, vinblastine, paracetamol, vitamin K, vitamin C.

Where the thermosensitive polymer comprises an active agent, it is preferably chemically bound to a carrier, which carrier is immobilised on the polymer.

The thermosensitive polymer may optionally also comprise one or more secondary active agents as described herein, optionally independently selected from an imaging agent (e.g. a fluorophore or a radio-labelled moiety) and a targeting agent (e.g. a receptor-binding protein, an antibody, a nucleic acid or a peptide).

### Carrier

The nanoparticle-polymer complex may comprise a carrier.

One possible function of the carrier may be to immobilise an active agent in the polymer in the nanoparticle-polymer complex. Accordingly, the term “carrier” is typically used to refer to a species which can bind to the active agent and to the polymer. When the carrier is present in the complex of the invention, it is immobilised on the polymer. By “immobilised on” is meant that the carrier is bound to the polymer.

Accordingly, it is preferred that the nanoparticle-polymer complex comprises a carrier. It is particularly preferred that the nanoparticle-polymer complex comprises a carrier and an active agent. For instance, the nanoparticle-polymer complex may comprise a carrier immobilised on the polymer and an active agent bound to the carrier. When the carrier is present in the complex of the invention, it is typically bound to the polymer and the active agent.

It should be noted that, herein, the term “carrier” may be used to refer both to the carrier species when immobilised in the complex of the invention and also the carrier species prior to immobilisation on the polymer. A carrier may or may not be bound to an active agent. Whether “carrier” refers to either or both of these forms will generally be clear form the context. However, where it is particularly important to differentiate between the carrier bound to the polymer and the carrier prior to incorporation into the complex, the term “carrier precursor” will be used. A carrier precursor may or may not be bound to an active agent.

When the polymer comprising an active agent bound to a carrier is caused to undergo a phase transition, typically the species released by the complex to the environment comprises or consists of the active agent bound to the carrier. Accordingly, a possible additional function of the carrier is to support the function of the active agent once it has been released. For instance, the carrier may be a species which improves the biocompatibility of the active agent or improves its solubility. Thus, it is preferred that the carrier may be a biocompatible species, such as a protein.

The carrier may form any kind of bond with the active agent. For instance, the carrier may be capable of forming one or more of a covalent bond, an ionic bond, a hydrogen bond, a polar-non-polar interaction or a Van der Waals interaction with the active agent. It may be preferred that the carrier is capable of forming weaker bonds with the active agent (such as Van der Waals interactions or hydrogen bonds) so that the carrier may more easily release the active agent into the environment, once the carrier has been released from the complex.

The carrier may also form any kind of bond with the polymer. For instance, the carrier (specifically the carrier precursor) may be capable of forming one or more of a covalent bond, an ionic bond, a hydrogen bond, a polar-non-polar interaction or a Van der Waals interaction with the polymer. It is advantageous for the carrier to form a strong bond with the polymer, in order to hold the active agent within the complex until it undergoes a phase transition from the solvated phase to the globular conformation. It is not necessary for the carrier to be released from the polymer when the active agent when the polymer undergoes a phase transition. The carrier may remain bound, and an active agent attached to the carrier may be released. Accordingly, in some embodiments the carrier is not reversibly attached to the polymer in the nanoparticle-polymer complex. It may for instance be bound to the polymer by a strong covalent bond or bonds.

On the other hand, it is also advantageous for the bond between the carrier and the polymer to be sufficiently weak that the carrier (possibly together with the active agent) is released from the complex when the polymer undergoes the said phase transition. Preferably, therefore, the carrier is reversibly attached to the polymer in the nanoparticle-polymer complex.

In a preferred aspect, therefore, the carrier is preferably bound to the polymer by a covalent bond and ideally by a weak covalent bond. A weak covalent bond is a covalent bond which can be broken under physiological conditions. Such a covalent bond may also be referred to as a reversible covalent bond. Examples of reversible covalent bonds include disulphide bridges (S—S bonds) and C═N bonds.

Other weak bonds which may be preferred are hydrogen bonds.

Accordingly, the nanoparticle-polymer complex of the invention preferably comprises a carrier wherein the carrier is bound to the polymer by one or more of a hydrogen bond, a disulphide bridge (S—S bond) or a C═N bond; preferably by a disulphide bridge or a C═N bond; most preferably by a disulphide bridge.

Accordingly, it is preferred that the carrier (specifically the carrier precursor) comprises one or more of a hydroxyl group; a thiol group (—SH); a carboxyl group —COR; or an amine group —NR₂; where each R (where present) is independently selected from H or C₁₋₆ alkyl, more preferably H or C₁₋₃ alkyl, most preferably H. Preferably the carrier (specifically the carrier precursor) comprises a thiol group (—SH), an aldehyde group —C(═O)H; or an amine group (—NH₂). Most preferably the carrier (specifically the carrier precursor) comprises a thiol group (—SH).

Preferably the carrier is a biocompatible protein. For instance, where the complex is intended for use in humans it may be preferred that the carrier is a protein which is approved for use in humans. Examples of the carrier are bovine serum albumin or human serum albumin, preferably human serum albumin where the complex is intended for use in humans.

### Active Agent

Generally, the nanoparticle-polymer complex of the invention comprises an active agent. As explained above, it is preferred that the complex comprises an active agent and a carrier.

Typically, the active agent is immobilised in the complex. By “immobilised” is meant that the active agent is bound to the complex. The active agent may be immobilised in the complex in various ways: the active agent may be bound to the iron oxide nanoparticle, or to the polymer, or to a carrier (which carrier is preferably immobilised on the polymer).

It should be noted that, herein, the term “active agent” may be used to refer both to the active agent species when immobilised in the complex of the invention and also the free active agent once released from the complex. The free active agent may or may not be bound to a carrier. Whether “active agent” refers to either or both of these forms will generally be clear from the context. However, where it is particularly important to differentiate between the active agent immobilised in the complex and the free active agent, the term “free active agent” will be used. A free active agent may or may not be bound to a carrier.

Where the active agent is bound to the iron oxide nanoparticle, it may be bound by, for instance, an ionic bond or a hydrogen bond. For instance, the active agent may be ionically bound to anionic oxygen species or OH groups at the surface of the iron oxide nanoparticle. In such embodiments, the active agent (particularly the free active agent) typically comprises an ionic moiety and/or a hydroxyl group.

Where the active agent is bound to the polymer, it may form any kind of bond with the active agent. For instance, the active agent (specifically the free active agent) may be capable of forming one or more of a covalent bond, an ionic bond, a hydrogen bond, a polar-non-polar interaction or a Van der Waals interaction with the polymer. It is advantageous for the active agent (specifically the free active agent) to be able to form a bond with the polymer, in order to hold the active agent within the complex until it undergoes a phase transition from the solvated phase to the globular conformation. On the other hand, it is also advantageous for the bond between the active agent and the polymer to be sufficiently weak that the active agent is released from the complex when the polymer undergoes the said phase transition. Preferably, therefore, where the active agent is bound to the polymer it is reversibly bound. Further preferably, where the active agent is bound to the polymer it is bound by a bond which can be broken under physiological conditions (such as a reversible covalent bond or a hydrogen bond), in order to ensure that the active agent is released on activation.

Accordingly, the active agent may be bound to the polymer by a covalent bond and ideally by a weak covalent bond. Alternatively the active agent may be bound to the polymer by a hydrogen bond or bonds. Thus, where the complex comprises a an active agent bound to the polymer, the active agent is preferably bound to the polymer by one or more of a hydrogen bond, a disulphide bridge (S—S bond) or a C═N bond; preferably by a disulphide bridge or a C═N bond; most preferably by a disulphide bridge.

Alternatively, the active agent may be incorporated into the complex by being bound to a carrier as described in the preceding section.

An active agent is a species having a desired chemical activity. Generally, an active agent has biological activity. An active agent may for instance have antimicrobial activity or therapeutic activity. Preferably, the active agent is a pharmaceutically active agent. The active agent may be for instance a chemotherapeutic agent, a thrombolytic agent, an antimicrobial agent or an anti-inflammatory agent; preferably the active agent is a chemotherapeutic agent.

Examples of chemotherapeutic agents are well known. Examples of anti-cancer treatments including chemotherapeutic agents are listed at https://www.cancerresearchthuk.org/about-cancer/cancer-in-general/treatment/cancer-drugs/drugs. The chemotherapeutic agent may for example be any of the individual agents appearing in the following list: Abiraterone Acetate, Abitrexate (Methotrexate), ABVD (i.e. a combination of Doxorubicin Hydrochloride (Adriamycin), Bleomycin, Vinblastine Sulfate and Dacarbazine), ABVE (i.e. a combination of Doxorubicin Hydrochloride, Bleomycin, Vincristine Sulfate and Etoposide), ABVE-PC (i.e. a combination of Doxorubicin Hydrochloride, Bleomycin, Vincristine Sulfate, Etoposide, Prednisone and Cyclophosphamide), AC (i.e. a combination of Doxorubicin Hydrochloride and Cyclophosphamide), AC-T (i.e. a combination of Doxorubicin Hydrochloride, Cyclophosphamide and Paclitaxel (Taxol)), Adcetris (Brentuximab Vedotin), ADE (i.e. a combination of Cytarabine (Ara-C), Daunorubicin Hydrochloride and Etoposide), Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alkeran for Injection (Melphalan Hydrochloride), Alimta (Pemetrexed Disodium), Aloxi (Palonosetron Hydrochloride), Ambochlorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Axitinib, Azacitidine, BEACOPP (i.e. a combination of Bleomycin, Etoposide, Doxorubicin Hydrochloride, Cyclophosphamide, Vincristine Sulfate (Oncovin), Procarbazine Hydrochloride and Prednisone), Becenum (Carmustine), Beleodaq (Belinostat), Belinostat, Bendamustine Hydrochloride, BEP (i.e. a combination of Bleomycin, Etoposide and Cisplatin (Platinol)), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, BuMel, Busulfan, Busulfex (Busulfan), Cabazitaxel, Cabometyx (Cabozantinib-S-Malate), Cabozantinib-S-Malate, CAF (i.e. a combination of Cyclophosphamide, Doxorubicin Hydrochloride (Adriamycin) and Fluorouracil), Campath (Alemtuzumab), Camptosar (Irinotecan Hydrochloride), Capecitabine, CAPDX (a combination of Capecitabine and Oxaliplatin), Carac (Fluorouracil—Topical), Carboplatin, CARBOPLATIN-TAXOL (a combination of Carboplatin and Paclitaxel), Carfilzomib, Carmubris (Carmustine), Carmustine, Carmustine Implant, Casodex (Bicalutamide), CEM (a combination of Carboplatin, Etoposide and Melphalan Hydrochloride), Ceritinib, Cerubidine (Daunorubicin Hydrochloride), Cetuximab, Chlorambucil, CHLORAMBUCIL-PREDNISONE (a combination of Chlorambucil and Prednisone), CHOP (a combination of Cyclophosphamide, Doxorubicin Hydrochloride (Hydroxydaunomycin), Vincristine Sulfate (Oncovin) and Prednisone), Cisplatin, Clafen (Cyclophosphamide), Clofarabine, Clofarex (Clofarabine), Clolar (Clofarabine), CMF (a combination of Cyclophosphamide, Methotrexate and Fluorouracil), Cobimetinib, Cometriq (Cabozantinib-S-Malate), COPDAC (a combination of Cyclophosphamide, Vincristine Sulfate (Oncovin), Prednisone and Dacarbazine), COPP (a combination of Cyclophosphamide, Vincristine Sulfate (Oncovin), Procarbazine Hydrochloride and Prednisone), COPP-ABV (a combination of Cyclophosphamide, Vincristine Sulfate, Procarbazine Hydrochloride, Prednisone, Doxorubicin Hydrochloride, Bleomycin and Vinblastine Sulfate), Cosmegen (Dactinomycin), Cotellic (Cobimetinib), Crizotinib, CVP (a combination of Cyclophosphamide, Vincristine Sulfate and Prednisone), Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Doxorubicin Hydrochloride, DTIC-Dome (Dacarbazine), Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enzalutamide, Epirubicin Hydrochloride, EPOCH (a combination of Etoposide, Prednisone, Vincristine Sulfate, Cyclophosphamide and Doxorubicin Hydrochloride), Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC (a combination of Fluorouracil, Epirubicin Hydrochloride, and Cyclophosphamide), Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI (a combination of Leucovorin Calcium (Folinic Acid), Fluorouracil and Irinotecan Hydrochloride), a combination of 5-fluorouracil, oxaliplatin and folinic acid (as used in FOXFIRE), FOLFIRI-BEVACIZUMAB (a combination of Leucovorin Calcium, Fluorouracil, Irinotecan Hydrochloride and Bevacizumab), FOLFIRI-CETUXIMAB (a combination of Leucovorin Calcium, Fluorouracil, Irinotecan Hydrochloride and Cetuximab), FOLFIRINOX (a combination of Leucovorin Calcium, Fluorouracil, Irinotecan Hydrochloride and Oxaliplatin), FOLFOX (a combination of Leucovorin Calcium, Fluorouracil and Oxaliplatin), Folotyn (Pralatrexate), FU-LV (a combination of Fluorouracil and Leucovorin Calcium), Fulvestrant, Gazyva (Obinutuzumab), Gefitinib, Gemcitabine Hydrochloride, Gemcitabine-Cisplatin combination, Gemcitabine-Oxaliplatin combination, Gemtuzumab, Ozogamicin, Gemzar (Gemcitabine Hydrochloride), Gilotrif (Afatinib Dimaleate), Gleevec (Imatinib Mesylate), Gliadel (Carmustine Implant), Glucarpidase, Goserelin Acetate, Halaven (Eribulin Mesylate), Herceptin (Trastuzumab), Hycamtin (Topotecan Hydrochloride), Hydrea (Hydroxyurea), Hydroxyurea, Hyper-CVAD (a combination of Cyclophosphamide, Vincristine Sulfate, Doxorubicin Hydrochloride (Adriamycin) and Dexamethasone), Ibrance (Palbociclib), Ibritumomab Tiuxetan, Ibrutinib, ICE (a combination of Ifosfamide, Carboplatin and Etoposide), Iclusig (Ponatinib Hydrochloride), Idamycin (Idarubicin Hydrochloride), Idarubicin Hydrochloride, Idelalisib, Ifex (Ifosfamide), Ifosfamide, Ifosfamidum (Ifosfamide), IL-2 (Aldesleukin), Imatinib Mesylate, Imbruvica (Ibrutinib), Imiquimod, Imlygic (Talimogene Laherparepvec), Inlyta (Axitinib), Interferon Alfa-2b Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan, Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kyprolis (Carfilzomib), Lanreotide Acetate, Lapatinib Ditosylate, Lenalidomide, Lenvatinib Mesylate, Lenvima (Lenvatinib Mesylate), Letrozole, Leucovorin Calcium, Leukeran (Chlorambucil), Leuprolide Acetate, Levulan (Aminolevulinic Acid), Linfolizin (Chlorambucil), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Lynparza (Olaparib), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitoxantrone Hydrochloride, Mitozytrex (Mitomycin C), MOPP (a combination of Mechlorethamine Hydrochloride, Vincristine Sulfate (Oncovin), Procarbazine Hydrochloride and Prednisone), Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Navelbine (Vinorelbine Tartrate), Necitumumab, Nelarabine, Neosar (Cyclophosphamide), Netupitant and Palonosetron Hydrochloride, Neupogen (Filgrastim), Nexavar (Sorafenib Tosylate), Nilotinib, Ninlaro (Ixazomib Citrate), Nivolumab, Nolvadex (Tamoxifen Citrate), Nplate (Romiplostim), Obinutuzumab, Odomzo (Sonidegib), OEPA (a combination of Vincristine Sulfate, Etoposide, Prednisone and Doxorubicin Hydrochloride), Ofatumumab, OFF (a combination of Oxaliplatin, Fluorouracil, Leucovorin Calcium (Folinic Acid)), Olaparib, Omacetaxine Mepesuccinate, Oncaspar (Pegaspargase), Ondansetron Hydrochloride, Ontak (Denileukin Diftitox), Opdivo (Nivolumab), OPPA (a combination of Vincristine Sulfate (Oncovin), Procarbazine Hydrochloride, Prednisone and Doxorubicin Hydrochloride (Adriamycin)), Osimertinib, Oxaliplatin, Paclitaxel, PAD (a combination of Bortezomib (PS-341), Doxorubicin Hydrochloride (Adriamycin) and Dexamethasone), Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV (a combination of Procarbazine Hydrochloride, Lomustine (CCNU) and Vincristine Sulfate), Pegaspargase, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Purixan (Mercaptopurine), Raloxifene Hydrochloride, Ramucirumab, Rasburicase, R-CHOP (a combination of Rituximab, Cyclophosphamide, Doxorubicin Hydrochloride, Vincristine Sulfate, and Prednisone), R-CVP (a combination of Rituximab, Cyclophosphamide, Vincristine Sulfate and Prednisone), Recombinant Interferon Alfa-2b, Regorafenib, R-EPOCH (a combination of Rituximab, Etoposide, Prednisone, Vincristine Sulfate, Cyclophosphamide and Doxorubicin Hydrochloride), Revlimid (Lenalidomide), Rheumatrex (Methotrexate), Rituxan (Rituximab), Rituximab, Rolapitant Hydrochloride, Romidepsin, Romiplostim, Rubidomycin (Daunorubicin Hydrochloride), Ruxolitinib Phosphate, Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V (a combination of Mechlorethamine Hydrochloride, Doxorubicin Hydrochloride, Vinblastine Sulfate, Vincristine Sulfate, Bleomycin, Etoposide and Prednisone), Stivarga (Regorafenib), Sunitinib Malate, Sutent (Sunitinib Malate), Sylatron (Peginterferon Alfa-2b), Sylvant (Siltuximab), Synovir (Thalidomide), Synribo (Omacetaxine Mepesuccinate), Tabloid (Thioguanine), TAC (a combination of Docetaxel (Taxotere), Doxorubicin Hydrochloride (Adriamycin) and Cyclophosphamide), Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF (Docetaxel (Taxotere), Cisplatin (Platinol) and Fluorouracil), Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC (a combination of Vincristine Sulfate, Dactinomycin (Actinomycin-D) and Cyclophosphamide), Vandetanib, VAMP (Vincristine Sulfate, Doxorubicin Hydrochloride (Adriamycin), Methotrexate and Prednisone), Varubi (Rolapitant Hydrochloride), Vectibix (Panitumumab), VeIP (a combination of Vinblastine Sulfate (Velban), Ifosfamide and Cisplatin (Platinol)), Velban (Vinblastine Sulfate), Velcade (Bortezomib), Velsar (Vinblastine Sulfate), Vemurafenib, Venclexta (Venetoclax), Venetoclax, Viadur (Leuprolide Acetate), Vidaza (Azacitidine), Vinblastine Sulfate, Vincasar PFS (Vincristine Sulfate), Vincristine Sulfate, Vinorelbine Tartrate, VIP (a combination of Etoposide (VePesid), Ifosfamide and Cisplatin (Platinol)), Vismodegib, Vistogard (Uridine Triacetate), Voraxaze (Glucarpidase), Vorinostat, Votrient (Pazopanib Hydrochloride), Wellcovorin (Leucovorin Calcium), Xalkori (Crizotinib), Xeloda (Capecitabine), XELIRI (a combination of Capecitabine (Xeloda) and Irinotecan Hydrochloride), XELOX (Capecitabine (Xeloda) and Oxaliplatin), Xgeva (Denosumab), Xtandi (Enzalutamide), Yervoy (Ipilimumab), Yondelis (Trabectedin), Zaltrap (Ziv-Aflibercept), Zarxio (Filgrastim), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib) or Zytiga (Abiraterone Acetate).

For instance, the active agent may be docetaxel, doxorubicin, etoposide, ifosfamide, paclitaxel, vinblastine, paracetamol, vitamin K, and vitamin C.

Preferred examples of the active agent are paclitaxel, vinblastine, paracetamol, vitamin K, vitamin C.

The nanoparticle-polymer complex of the invention may comprise a secondary active agent. A secondary active agent is typically an agent which has a function complementary to the function of the first active agent. The secondary active agent is typically incorporated into the complex as described above in connection with the active agent: it may be bound to the iron oxide nanoparticle, or the polymer, or to a carrier.

The secondary active agent may be an imaging agent. Suitable examples of the imaging agent include a fluorophore, or a radioisotopically-labelled species. An imaging agent is a useful addition as it may be desired to monitor the location of the complex within a system such as pipe or a patient.

The secondary active agent may be a targeting agent. A targeting agent is an agent which can assist in localising the complex at a site of interest, for instance by binding to the said site. A targeting agent is particularly useful where the complex is to be used in a method of therapy, for instance an in vivo method of therapy. Suitable examples of a targeting agent include a receptor-targeting moiety, e.g. a receptor-binding protein such as an antibody; a nucleic acid, or a peptide.

The nanoparticle-polymer complex of the invention may contain one active agent. Alternatively the nanoparticle-polymer complex may comprise two or more active agents, for example three or more active agents. Where the nanoparticle-polymer complex comprises more than one active agent, they may be referred to respectively as the first active agent, the second active agent, and so on.

Where the nanoparticle-polymer complex comprises more than one active agent, for example a first active agent and a second active agent, the active agents may act in a complementary fashion. For instance, the active agents may act together to produce a synergistic effect. By way of example the nanoparticle-polymer complex may comprise a first chemotherapeutic agent and a second chemotherapeutic agent.

The nanoparticle-polymer complex may comprise an active agent and a secondary active agent. The nanoparticle-polymer complex may comprise an active agent and a secondary active agent. For instance, the nanoparticle-polymer complex may comprise a pharmaceutically active agent and a targeting agent or a pharmaceutically active agent and an imaging agent. In a preferred embodiment, the nanoparticle-polymer complex comprises a chemotherapeutic agent and a targeting agent.

The complex may comprise an active agent and two or more secondary active agents.

Where the nanoparticle-polymer complex comprises two or more active agents (or an active agent and one or more secondary active agents), each agent may independently be bound to the iron oxide nanoparticle, the polymer or, where present, a carrier.

Some exemplary instances of the complex comprising a carrier and an active agent will now be described. In each instance, the complex comprises a carrier immobilised on the polymer and an active agent bound to the carrier (and hence to the polymer). Preferably, the active agent is a chemotherapeutic agent. In each instance, it is preferred that the phase change unit comprises at least about 40% by weight of the polymer; the binding unit comprises at least 1% by weight of the polymer; and the LCST-adjusting unit comprises at least about 1 wt % of the polymer. More preferably, the phase change unit comprises about 40 wt % to 99 wt %; the binding unit comprises about 1 wt % to 40 wt %; and the LCST-adjusting unit comprises about 1 wt % to about 50 wt % of the polymer. Still more preferably, the phase change unit comprises about 50 wt % to 95 wt %; the binding unit comprises about 5 wt % to 35 wt %; and the LCST-adjusting unit comprises about 5 wt % to about 45 wt % of the polymer.
The polymer comprises a phase-change repeating unit of formula (X), a binding repeating unit of formula (Y) and an LCST-adjusting unit of formula (Z).
Preferably, the polymer comprises a phase change repeating unit of formula (XA), (XB) or (XC); a binding unit of formula (YA), (YB), (YC) or (YD) and an LCST-adjusting repeating unit of formula (ZA) or (ZB).
In a particularly preferred embodiment, the polymer comprises a phase change repeating unit of formula (XA), a binding unit of formula (YA) or (YD) and an LCST-adjusting repeating unit of formula (ZA) or (ZB).
For example, the polymer may comprise a phase change repeating unit of formula (XA), a binding unit of formula (YA) and an LCST-adjusting unit of formula (ZA). Alternatively, the polymer may comprise a phase change repeating unit of formula (XA), a binding unit of formula (YD) and an LCST-adjusting unit of formula (ZB).

In further exemplary embodiments, the complex comprises a carrier immobilised on the polymer and an active agent bound to the carrier (and hence to the polymer). In all instances, it is preferred that the phase change unit comprises at least about 25% by weight of the polymer; the binding unit (where present) comprises at least 1% by weight of the polymer; and the LCST-adjusting unit comprises at least about 5 wt % of the polymer. More preferably, the phase change unit comprises about 25 wt % to 90 wt %; the binding unit comprises about 1 wt % to 40 wt %; and the LCST-adjusting unit comprises about 5 wt % to about 70 wt % of the polymer. Still more preferably, the phase change unit comprises about 30 wt % to 60 wt %; the binding unit comprises about 5 wt % to 35 wt %; and the LCST-adjusting unit comprises about 10 wt % to about 65 wt % of the polymer. Still more preferably, the phase change unit comprises about 35 to 50 wt %; the binding unit comprises about 10 to 30 wt %; and the LCST-adjusting unit comprises about 20 to 60 wt % of the polymer. Further preferably, the phase change unit comprises about 35 wt % to 45 wt %; the binding unit comprises about 15 wt % to 25 wt %; and the LCST-adjusting unit comprises about 40 wt % to about 55 wt % of the polymer. Most preferably, the phase change unit comprises about 35 to 40 wt %; the binding unit comprises about 15 to 20 wt %; and the LCST-adjusting unit comprises about 45 to 50 wt % of the polymer.
The polymer comprises a phase-change repeating unit of formula (X), a binding repeating unit of formula (Y) and an LCST-adjusting unit of formula (Z).
For instance, the polymer comprises a phase change repeating unit of formula (XA), (XB) or (XC); a binding unit of formula (YA), (YB), (YC), (YD) or (YE) and an LCST-adjusting repeating unit of formula (ZA) or (ZB).
In a particularly preferred embodiment, the polymer may comprise a phase change repeating unit of formula (XA), a binding unit of formula (YE) or (YD) and an LCST-adjusting repeating unit of formula (ZB).

### Iron Oxide Nanoparticle

The complex of the invention comprises an iron oxide nanoparticle. This may be referred to herein as “the nanoparticle” or “the iron oxide nanoparticle”.

Typically the iron oxide nanoparticle is magnetic, meaning that the nanoparticle has one or more magnetic domains. The iron oxide nanoparticle may be ferromagnetic. A ferromagnetic nanoparticle is one having multiple magnetic domains whose magnetic moments are aligned. Alternatively the iron oxide nanoparticle may be ferromagnetic, wherein the nanoparticle has multiple magnetic domains whose moments have different magnitudes and are opposed. More preferably, the iron oxide nanoparticle may be superparamagnetic. A superparamagnetic nanoparticle is an example of a ferromagnetic or ferromagnetic material whose magnetic domains are sufficiently small that they can spontaneously change direction; however, the domains become aligned in the presence of an external magnetic field. It is preferred that the iron oxide nanoparticle is superparamagnetic as superparamagnetic nanoparticles generate heat particularly efficiently when exposed to an alternating magnetic field.

The iron oxide nanoparticle may comprise any iron oxide such as Fe₂O₃ or Fe₃O₄, but preferably comprises Fe₃O₄. For instance the iron oxide nanoparticle may comprise or consist of magnetite. Preferably, the iron oxide nanoparticle may consist of magnetite.

The iron oxide nanoparticle may comprise or consist of crystalline iron oxide. For instance, the iron oxide nanoparticle may comprise or consist of a single crystal of iron oxide, such as a single crystal of Fe₃O₄, i.e. a single crystal of magnetite.

The iron oxide nanoparticle is typically about 1-100 nm in diameter, preferably about 2-50 nm, more preferably about 2-20 nm in diameter, particularly preferably about 5 to 15 nm in diameter.

### Composition Comprising Plurality of Nanoparticle Polymer Complexes

The nanoparticle-polymer complex may be formulated as a composition. A composition comprising the nanoparticle-polymer complex is typically a liquid composition. The composition may alternatively be a solid or semi-solid composition, for instance a gel.

A composition comprising the nanoparticle-polymer complex of the invention typically comprises a nanoparticle-polymer complex together with one or more excipients. Generally the excipient(s) are pharmaceutically acceptable excipients. Examples of suitable excipients include water, a buffer, a stabilising agent, or a preservative. A particularly suitable excipient is phosphate buffered saline. Typically, the composition is an aqueous solution comprising water and the nanoparticle-polymer complex.

The composition typically comprises a plurality of nanoparticle-polymer complexes. Each complex within the plurality of nanoparticle-polymer complexes is independently as described above. Accordingly, each complex within the said plurality of the nanoparticle-polymer complexes may be of the same type (for instance, comprising the same active agent and the same polymer). Alternatively, the composition may comprise different kinds of nanoparticle-polymer complexes, comprising different active agents and/or different polymers.

Generally, therefore, the composition comprises:
(i) a first nanoparticle-polymer complex comprising an iron oxide nanoparticle, a first active agent and a first polymer capable of undergoing a phase change at a first predetermined temperature; and
(ii) a second nanoparticle-polymer complex comprising an iron oxide nanoparticle, a second active agent and a second polymer capable of undergoing a phase change at a second predetermined temperature.

Optionally, the composition further comprises one or more excipients. Preferably the composition does comprise an excipient, typically water.

In said composition, the first polymer and second polymer may be the same or different; and/or the first active agent and the second active agent may be the same or different; and/or the first predetermined temperature and second predetermined temperature may be the same or different.

The first nanoparticle-polymer complex may optionally comprise a first carrier, and the second nanoparticle-polymer complex may optionally comprise a second carrier; the said first carrier and the said second carrier may be the same or different.

Thus, in one example the composition may comprise a first nanoparticle-polymer complex comprising a first active agent and a second nanoparticle-polymer complex comprising a second active agent, the first active agent being different to the second active agent. Similarly, the composition may comprise a first nanoparticle-polymer complex comprising a first polymer and a second nanoparticle-polymer complex comprising a second polymer, the first polymer being different to the second polymer.

Each nanoparticle-polymer complex may be optimised for delivery of the active agent therein.

The first active agent and the second active agent may have activities which complement one another; for instance, they may act synergistically when released.

The composition as described above is particularly useful where it is desirable to simultaneously administer two active agents while preventing them from interacting. This is the case where it is desired to administer vitamin C and vitamin K simultaneously. In such cases, the first pre-determined temperature is similar to the second pre-determined temperature; for instance, they may differ by 2° C. or less; preferably by 1° C. or less. Accordingly, in a preferred example, the composition comprises:
(i) a first nanoparticle-polymer complex comprising an iron oxide nanoparticle, a first active agent which is vitamin C and a first polymer capable of undergoing a phase change at a first predetermined temperature; and
(ii) a second nanoparticle-polymer complex comprising an iron oxide nanoparticle, a second active agent which is vitamin K and a second polymer capable of undergoing a phase change at a second predetermined temperature.

Preferably, the composition further comprises one or more pharmaceutically acceptable excipients. Further preferably, the first pre-determined temperature is similar to the second pre-determined temperature.

### Article

The nanoparticle-polymer complex of the invention can be used in the form of a composition as described above (for instance, in solution) but may be provided as part of an article. Accordingly, the invention provides an article comprising a nanoparticle-polymer complex as described herein.

The nanoparticle-polymer complex of the invention may be coated onto a surface of an article. Alternatively or additionally, the nanoparticle-polymer complex may be present within an article. For instance, the article may comprise a compartment containing the nanoparticle-polymer complex (for instance in solution). Alternatively the nanoparticle-polymer complex may be localised within the article, for instance embedded in a gel or in a scaffold. The skilled person will appreciate a wide variety of methods known in the art for incorporating polymeric particles within an article which may be applicable to the present invention.

By way of example, the article may be a medical device suitable for insertion into the human or animal body. Alternatively, the article may be an article suitable for use in a non-medical process.

One example of an article according to the invention is a stent, which comprises (for instance is coated with, or comprises a layer containing) a nanoparticle-polymer complex as described herein. In this embodiment, the nanoparticle polymer-complex preferably comprises an active agent, particularly preferably a thrombolytic agent.

Another example of an article according to the invention is a component of a water treatment system, which comprises (for instance is coated with, or comprises a layer containing) a nanoparticle-polymer complex as described herein. In this embodiment, the nanoparticle polymer-complex preferably comprises an active agent, particularly preferably an antimicrobial agent. A similar example of an article according to the invention is a component of an oil pipe system which comprises (for instance is coated with, or comprises a layer containing) a nanoparticle-polymer complex as described herein, preferably comprising an antimicrobial agent.

### Process for Producing a Nanoparticle Polymer Complex

The nanoparticle-polymer complex as described herein may be produced by providing an iron oxide nanoparticle and generating a polymer bound to the iron oxide nanoparticle. The iron oxide nanoparticle and the polymer are as described above.

The process may comprise providing an iron oxide nanoparticle and providing a polymer, and then subsequently attaching the polymer to the iron oxide nanoparticle. More usually, however, the process comprises providing an iron oxide nanoparticle and then providing a polymer bound to the iron oxide nanoparticle. This latter process is preferred as it is generally easier to prepare the polymer “in situ” rather than trying to manipulate it to form a complex as described herein.

Accordingly, a typical process for producing a nanoparticle-polymer complex as described herein may comprise:
a) providing an iron oxide nanoparticle;
b) providing a plurality of monomers; and
c) polymerising the monomers to provide a polymer attached to the iron oxide nanoparticle.

The “plurality of monomers” comprises a phase change monomer as described herein. Accordingly, the plurality of monomers typically comprises a monomer selected from N-isopropylacrylamide, N-vinyl caprolactam, and methyl vinyl ether. Generally, the plurality of monomers also comprises an “LCST-adjusting monomer” as described herein. Accordingly, the plurality of monomers may comprise a monomer selected from acrylic acid and acrylamide. Typically, the “plurality of monomers” also comprises a binding monomer as described herein. Accordingly, the plurality of monomers typically comprises a monomer selected from methacrolein, allyl mercaptan, acrolein, crotonaldehyde and 2-pentenal (particularly trans-2-pentenal or methacrolein).

Thus, in a preferred embodiment, the plurality of monomers comprises: a phase change monomer selected from N-isopropylacrylamide, N-vinyl caprolactam, and methyl vinyl ether, preferably N-isopropylacrylamide; an LCST-adjusting monomer selected from acrylic acid and acrylamide, preferably acrylamide; and a binding monomer selected from methacrolein, allyl mercaptan, acrolein, crotonaldehyde and 2-pentenal; preferably methacrolein, allyl mercaptan and acrolein; more preferably methacrolein or acrolein; most preferably methacrolein.

The plurality of monomers may be referred to as a “monomer mixture”.

Typically, the plurality of monomers comprises at least 40% by weight of the phase-change monomer (compared to the total weight of the plurality of monomers). For instance, the plurality of monomers may contain from about 40 wt % to about 99 wt %; or about 50 wt % to about 95 wt %; or about 55 wt % to about 90 wt %; or about 60 wt % to about 85 wt % by weight of the phase-change monomer.

Usually, the plurality of monomers comprises at least 0.1 wt % of the binding monomer. For instance, the plurality of monomers may comprise at least 0.5 wt % or at least 1 wt % of the binding monomer. In some embodiments the plurality of monomers comprises from about 1 wt % to about 40 wt %; preferably about 5 wt % to 35 wt %, for example about 10 wt % to 30 wt % or 15 wt % to 25 wt % of the binding monomer. For instance, the plurality of monomers may comprise about 20 wt % of the binding monomer.

Usually, the plurality of monomers comprises at least 0.1 wt % of the LCST-adjusting monomer, e.g. at least 1 wt % of the LCST-adjusting monomer. In some embodiments, the plurality of monomers comprises about 1 wt % to about 50 wt %, generally about 5 wt % to about 45 wt %, for example about 10 wt % to about 40 wt % of the LCST-adjusting monomer.

For example, the plurality of monomers may comprise at least 25% by weight of N-isopropylacrylamide; at least 1% by weight of methacrolein; and at least 5 wt % of acrylamide;

preferably the plurality of monomers may comprise 25 wt % to 90 wt % by weight of N-isopropylacrylamide; 1 to 40% by weight of methacrolein; and 5 to 70% by weight of acrylamide;
more preferably the plurality of monomers may comprise 30 wt % to 60 wt % by weight of N-isopropylacrylamide; 5 to 35% by weight of methacrolein; and 10 to 65% by weight of acrylamide;
still more preferably the plurality of monomers may comprise 35 wt % to 50 wt % by weight of N-isopropylacrylamide; 10 to 30% by weight of methacrolein; and 20 to 60% by weight of acrylamide;
still more preferably the plurality of monomers may comprise 35 wt % to 45 wt % by weight of N-isopropylacrylamide; 15 to 25% by weight of methacrolein; and 40 to 55% by weight of acrylamide;
most preferably the plurality of monomers may comprise 30 wt % to 40 wt % by weight of N-isopropylacrylamide; 15 to 20% by weight of methacrolein; and 45 to 50% by weight of acrylamide.

The plurality of monomers may also comprise an anchoring monomer. The anchoring monomer are as described above. For instance, a suitable example of the anchoring monomer is DDMAT.

Alternatively, the anchoring monomer may initially be attached to the iron oxide nanoparticle (e.g. bound, for instance by ionic bonding or hydrogen bonding as discussed above). Thus, a preferred process for producing a nanoparticle-polymer complex as described herein comprises:
a) providing an iron oxide nanoparticle with an anchoring monomer attached thereto;
b) providing a plurality of monomers; and
c) polymerising the monomers to provide a polymer attached to the anchoring monomer.

The step of providing an iron oxide nanoparticle, step (a), is a process which is well-described in the art (for instance in “Co-precipitation in aqueous solution synthesis of magnetite nanoparticles using iron(III) salts as precursors”, M. Khalil, Arabian Journal of Chemistry, Vol. 8, Issue 2, 2015, pp 279-284). It is common to provide the iron oxide nanoparticle in stabilised form. For instance, step (a) may comprise providing a ligand-stabilised iron oxide nanoparticle, e.g. stabilised by lauric acid or anion thereof. In such a method, the process may further comprise performing an ion-exchange reaction to attach the anchoring monomer to the iron oxide particle. In other embodiments, the iron oxide nanoparticle may be produced with the anchor monomer already attached; this may be achieved by synthesising the iron oxide nanoparticle in the presence of the anchoring monomer.

In the processes for producing a nanoparticle-polymer complex, the step of polymerising the monomers may comprise initiating polymerisation. For instance, the process may comprise providing a polymerisation initiator (which may be included in the monomer mixture). Initiating polymerisation may alternatively or additionally comprise initiating polymerisation by irradiating the plurality of monomers, for example with heat or UV light.

The polymerisation step, step (c), is preferably a RAFT polymerisation method. It is preferred, therefore, that the anchoring monomer is a RAFT agent, such as DDMAT. A RAFT agent is an agent comprising a —C(═S)—S— moiety. RAFT polymerisation is a preferred polymerisation process as it is a controlled process.

The process of producing a nanoparticle-polymer complex may also comprise incorporating a carrier and/or an active agent in to the complex. Accordingly, the processes described above may further comprise one or more of:
b)(i) attaching an active agent to the iron oxide nanoparticle prior to step (b) or (c)
d) attaching a carrier as described above to the polymer and optionally further attaching an active agent to the carrier;
e) attaching an active agent to the polymer.

Where the method comprises step (d) or step (e), the attachment of the carrier or active agent to the polymer produces an active agent and/or carrier immobilised on the polymer as described above.

### Method of Releasing an Active Agent

The invention provides a method of releasing an active agent, which method comprises:
a) providing a nanoparticle-polymer complex comprising an active agent and an iron oxide nanoparticle bound to a polymer capable of undergoing a phase change at a predetermined temperature; and
b) exposing the nanoparticle-polymer complex to an alternating magnetic field and thereby heating the iron oxide nanoparticle, causing the phase change to occur and active agent to be released.

The nanoparticle-polymer complex employed in the said method is a nanoparticle-polymer complex as described herein, comprising an active agent. Accordingly, step (a) comprises providing a nanoparticle-polymer complex as described herein, comprising an active agent.

Step (b) comprises exposing the nanoparticle-polymer complex to an alternating magnetic field. When an iron oxide nanoparticle as described herein is exposed to an alternating magnetic field, the nanoparticle is heated and in turn heats its surroundings, raising the local temperature. The mechanisms by which the magnetic field heats the nanoparticle are described in detail in “Magnetic nanoparticle-based therapeutic treatments for thermo-chemotherapy treatment of cancer”, Nanoscale, 2014, 6, 11553.

Exposure of the nanoparticle-polymer complex to an alternating magnetic field is typically achieved in one of two ways. One way is to position the iron oxide nanoparticle in or near to the coil of an electromagnet, and to apply a high frequency alternating voltage to the electromagnet. The resulting high frequency current through the coil generates a strong magnetic field within and around the coil. The magnetic field will alternate at the frequency of the alternating voltage. This field can penetrate almost any material with few exceptions. Accordingly, the iron oxide nanoparticles in or near to the electromagnet will interact with the magnetic field, even if the iron oxide nanoparticles are themselves inside another material (for instance a human or animal tissue).

Accordingly, the step of exposing the complex to an alternating magnetic field may comprise positioning the complex near to or within (preferably within) the coils of an electromagnet and applying a high frequency alternating voltage to the electromagnet.

The frequency of the alternating voltage is from 100 kHz to 1000 kHz.

Another method of exposing the nanoparticle-polymer complex to an alternating magnetic field is to irradiate the complex with radio waves. By “radio-waves” is meant “radio-frequency electromagnetic radiation”. The oscillating magnetic field component of the radio waves will interact with the iron oxide nanoparticle and heat it, and thus heat the complex. Any suitable source of radio waves may be used. Generally radio-waves have a frequency of 100 kHz to 1000 kHz.

Accordingly, the step of exposing the complex to an alternating magnetic field may comprise irradiating the iron oxide nanoparticles with radio waves.

As is clear from the above, where the complex is exposed to an alternating magnetic field, the frequency of the alternating magnetic field is from 100 kHz to 1000 kHz.

The maximum field strength of the alternating magnetic field is typically from 10 to 100 kA/m.

Exposure of the nanoparticle-polymer complex to an alternating magnetic field causes heating of the iron oxide nanoparticle. This in turn causes heating of the complex comprising the iron oxide nanoparticle and may also cause heating of the surrounding environment in the vicinity of the complex. Accordingly, step (b) comprises heating the complex.

Typically, step (b) comprises heating the complex from a temperature below the pre-determined temperature to a temperature above the pre-determined temperature. For instance, step (b) may comprise heating the complex to a temperature of 37° C. or more, preferably to a temperature of 39° C. or more, most preferably to a temperature of 41° C. or more.

As explained above, in some embodiments it is preferred to avoid excessive heating of the environment surrounding the iron oxide nanoparticles. Accordingly, in some embodiments step (b) comprises heating the local environment to a temperature of no more than 44° C., preferably to a temperature of no more than 42° C. Thus, in a preferred embodiment the process comprises heating the complex from a temperature of about 37° C. to a temperature of from about 39° C. to about 42° C.

In some embodiments, however, it may be desired to use the heating of the iron oxide nanoparticles to heat the environment surrounding the iron oxide nanoparticles. An example of such a method is a hyperthermic method. Accordingly, in some embodiments step (b) comprises heating the complex to a temperature of 42° C. or more, preferably to a temperature of 43° C. or more, for example to a temperature of 45° C. or more. This is typical where the method is a method of hyperthermic treatment of the human or animal body. Thus, in a preferred embodiment the process comprises heating the complex from a temperature of about 37° C. to a temperature of from about 39° C. to 50° C., for example to a temperature of about 41° C. to 48° C. or to a temperature of from about 43° C. to about 45° C.

Especially, step (b) comprises heating the complex to a temperature of from about 39° C. to about 45° C.; preferably from about 40° C. to about 43° C.; most preferably from about 41° C. to about 42° C.

As explained in detail above, the phase change of the polymer is a change from a solvated phase to a globular phase which is insoluble or has a very low solubility in aqueous solution. Accordingly, causing the polymer to undergo a phase change in step (b) involves causing the polymer to become less soluble in aqueous solution, for instance to become insoluble in aqueous solution. Thus, causing the polymer to undergo a phase change causes the polymer to reduce in size, i.e. to contract, which may be referred to as “shrinking” or “collapsing”. Thus, step (b) may comprise collapsing the polymer.

The process causes the active agent to be released. By this is meant that the active agent is released to the surrounding environment of the complex; in other words, the active agent is released from the complex and enters the environment. The surrounding environment may be a biological environment, for instance a human or animal body (preferably a human body). For instance, the process may comprise releasing the active agent in vivo. In other aspects of the method, the surrounding environment may be a non-biological environment such as a pipe (e.g. an oil pipe or water pipe).

Typically, in the method, the nanoparticle-polymer complex is present in a solution, generally in an aqueous solution.

Step (b) may cause the active agent to be released in a variety of different ways. As explained above, where the active agent is bound to the iron oxide nanoparticle, step (b) may cause the active agent to become exposed to the environment, and the active agent may subsequently be released to the environment (for instance by ion exchange).

Alternatively, where the active agent is immobilised within the polymer, the contraction of the polymer may expel the active agent from the polymer.

Preferably, the complex comprises a carrier immobilised on the polymer and an active agent bound to the carrier, and step (b) involves causing the carrier to dissociate from the polymer.

For instance, where the active agent is bound to the polymer (directly or via a carrier) the contraction of the polymer may cause breakage of the bond(s) between the active agent and the polymer or the carrier and the polymer. As explained above, the active agent or, where present, the carrier, is preferably bound to the polymer by a reversible covalent bond; in some embodiments, therefore, step (b) comprises breaking a reversible covalent bond between the active agent and the polymer or the carrier and the polymer.

As explained above, in preferred embodiments, the complex comprises an active agent or a carrier (preferably a carrier) bound to the polymer by one or more of a C═N bond, an S—S bond or a hydrogen bond; most preferably by an —S—S— bond. In such cases, step (b) may comprise breaking said C═N bond, S—S bond or hydrogen bond.

In other embodiments, however, step (b) may comprise breaking a bond other than the bond which attaches the active agent or carrier to the polymer. For instance, where the complex comprises a carrier bound to an active agent, step (b) may comprise breakdown of the carrier at a different site (for instance a different chemical bond) and hence release of the active agent.

Step (b) may cause all or some of the active agent present in the complex to be released from the complex. For instance, at least 5% of the active agent present in the complex may be released in step (b). Typically at least 10% of the active agent present in the complex may be released in step (b). Up to 100% of the active agent may be released from the complex in step (b), for instance up to 95% or up to 90%. These percentages are typically percentages by weight of the total amount of active agent in the complex.

Where not all of the active agent is released during step (b), the process can be repeated to release further active agent at a later time point. For instance, where step (b) cause up to 95% or more usually up to 90% or up to 80% of the active agent to be release, the process may be repeated.

Accordingly, the method may further comprise repeating step (b) one or more times. For instance, the process may comprise repeating step (b) after a delay so as to cause a further release of the active agent at a second time point. The process may comprise
c) ceasing exposing the nanoparticle-polymer complex to the alternating magnetic field; and
d) repeating step (b) after a time delay.

The time delay may be short (for instance a few seconds or less). However, the time delay is not particularly limited and will depend on the situation. For instance, the time delay may be up to a week or up to a month or up to six months.

Step (b) may be performed simultaneously with step (a), immediately after step (a), or at a delay after step (a). Generally, step (b) is performed immediately after step (a) or at a delay after step (a). Where step (b) is performed after a delay, the delay may vary from a short delay such as a few seconds up to hours, weeks, months or years after step (a). For instance step (b) may be performed from 0.1 seconds to 10 years after step (a). An advantage of the complex is that it can securely bind the active agent without releasing it over these longer delays. An example of a situation where it is useful to allow a longer delay is in the case of a cancer which has spread over a long period, meaning that the complex may be re-used to release the active agent at a new tumour location. Typically, however, the delay is up to 1 week, for instance from 1 second to 1 week.

For instance, where step (a) comprises administering the complex to a patient (by injection or other means), step (b) may be performed simultaneously with, immediately after or at a delay after that administration. As explained above, the delay may be up to several years (for instance up to 10 years); typically the delay is rather less than that and may be of the order of seconds, minutes, hours or days.

### Localisation of Complex

Step (b) of the method of releasing an active agent causes active agent to be released at a particular locus. This is the locus where the magnetic field interacts with the iron oxide nanoparticle in the complex. This locus is referred to as the active agent release site. This locus is significant because the locus requires both the alternating magnetic field and the iron complex to be present. For example, where a complex as described herein is distributed throughout the system and a part of that system is exposed to an alternating magnetic field, release of the active agent will occur only at the active agent release site.

It is commonly a desirable object of the method of releasing an active agent to release the active agent only at a specific active agent release site. For instance, where the active agent is to be released in a system, it may be desirable to release the active agent only at a particular part of the system. For instance, where it is desired to treat an accumulation of microbes in a water pipe or oil pipe, it would be preferable to release the active agent only at the site of the accumulation of microbes. Similarly, where the method is a method of treatment of the human or animal body, it may be desirable to release the active agent only at a specific site such as a tumour in order to minimise exposure of the surrounding tissue to the active agent.

Active agent release at a particular locus within a system can be achieved in two ways.

One way is to distribute the complex throughout the entire system in step (a) of the method, and then to expose only the desired active agent release site to an alternating magnetic field in step (b). This method is convenient as it does not require any targeting of the complex to a particular site within the system. However, for reasons of cost (among others) it is generally desirable to minimise the amount of the complex introduced to the system. This is particularly important where the system is a biological environment such as the human or animal body and introduction of the complex to the system may have unwanted side effects.

Accordingly, step (a) preferably comprises localising the nanoparticle-polymer complex at the active agent release site. This reduces the amount of complex that must be supplied to a system in order to achieve the desired dosage at the active agent release site.

For instance, step (a) may involve providing the nanoparticle-polymer complex directly to an active agent release site (within a system). By way of example, step (a) may comprise injecting the nanoparticle-polymer complex at the active agent release site, for instance into a tumour.

Alternatively the complex may be provided elsewhere (within the system) and step (a) may comprise localising the nanoparticle-polymer complex at a locus.

The magnetic properties of the iron oxide nanoparticle may be exploited to localise the complex using a magnet. Localisation of the complex can be performed remotely, using a magnet or magnets positioned at or near the active agent release site, but outside the system containing the complex. For instance where the nanoparticle-polymer complex is present in a human or animal body, the complex may be positioned at the site (e.g. a tumour) by a magnet outside the body. The complex may be introduced to the human or animal body at a site other than the active agent release site, for instance by injection. The complex may then be then positioned over time by providing a magnet external to the body at or near the active agent release site, which localises the complex at said site.

Localisation of the complex using a magnet(s) is of course equally applicable to non-biological systems. For instance, where the nanoparticle-polymer complex is present in a system such as a pipe (e.g. in an oil pipe or a water pipe) the nanoparticle-polymer complex may be introduced to the system as a position other than the active agent release site (for instance at an end of the pipe) and localised at the desired site by a magnet(s) present outside the system (e.g. outside the pipe).

The above-described methods of localisation of the complex using magnets may be used after release of the active agent to remove the complex from the system. Accordingly, the method may comprise removing the complex from the active agent release site after steps (a) and (b) (and optionally also steps (c) and (d)) have been performed. Preferably the complex is removed from the active agent release site using a magnet(s).

It is not always necessary to perform an active localisation step as the complex may become localised at the active agent release site without intervention. This may be referred to as “passive localisation”, as opposed to an active localisation process requiring action. This can be achieved in various ways, a few examples of which are described below.

In a non-biological system (for example an oil pipe or a water pipe), the nanoparticle-polymer complex may move to the desired active agent release site under gravity; or may be moved there by the flow of fluid through the pipe.

In a biological system such as the human or animal body, the nanoparticle-polymer complex may accumulate at the desired site (e.g. the site of a tumour) by the enhanced permeability and retention effect (EPR).

In a biological system such as the human or animal body, the nanoparticle-polymer complex may be modified to ensure that it accumulates at a desired active agent release site in the body by inclusion of a targeting agent as described above.

### Methods of Therapy

Typically the method of release of an active agent as described herein is a method of treatment of the human or animal body. The method of treatment is typically a method of therapy. Accordingly the invention provides a method as described herein which is a method of treatment or prevention of a disorder in a subject, the method comprising
a) administering a nanoparticle-polymer complex comprising an active agent and an iron oxide nanoparticle bound to a polymer capable of undergoing a phase change at a predetermined temperature to the subject; and
b) exposing the nanoparticle-polymer complex to an alternating magnetic field and thereby heating the iron oxide nanoparticle, causing the phase change to occur and active agent to be released.

In particular, the invention provides a method as described herein which is a method of treatment or prevention of a disorder in a subject, the method comprising
a) administering a composition comprising a pharmaceutically acceptable excipient and a nanoparticle-polymer complex comprising an active agent and an iron oxide nanoparticle bound to a polymer capable of undergoing a phase change at a predetermined temperature to the subject; and
b) exposing the nanoparticle-polymer complex to an alternating magnetic field and thereby heating the iron oxide nanoparticle, causing the phase change to occur and active agent to be released.

The subject is typically a human or an animal, preferably a human. The subject may be a patient, for instance a patient in need of the active agent. The patient may be suffering from or at risk of suffering from the disorder. For instance, the patient may have cancer or may have had cancer. For instance, the patient may have or may have had a solid cancerous tumour. One example is a tumour of the liver such as a liver metastasis; however, the tumour may be a solid tumour in any part of the body.

The patient may be an adult or a child. In some embodiments, the patient is a child and the cancer is a childhood cancer.

Accordingly, the method may be a method of treatment of cancer. In this embodiment, the subject is preferably a subject with cancer. Similarly, the active agent is typically a chemotherapeutic agent. The cancer is generally a solid cancer, rather than a blood cancer. Thus, the method of treatment of cancer is generally a method of treatment of a solid cancer, i.e. a cancerous tumour. By way of non-limiting example, the cancer may be lung cancer, breast cancer, colorectal cancer, prostate cancer, skin cancer, stomach cancer, liver cancer, or pancreatic cancer.

Where the method of treatment is a method of treatment of cancer, the active agent is generally a chemotherapeutic agent; however, this is not necessary. The active agent may have other activity, for instance as an analgesic, an anti-inflammatory, or to assist the function of another active agent (such as a chemotherapeutic agent). In one example, where the cancer is liver cancer (for instance liver metastasis), the active agent may be paracetamol.

Administering the complex in step (a) may comprise providing the nanoparticle-polymer complex to the subject by any suitable means. The complex may be provided by injection. The method may for instance comprise administering the complex to the subject by intravenous injection and then localising the active agent at the active agent release site by one or more of the active or passive localisation methods described herein. Alternatively, the method may comprise administering the complex to the active agent release site, for instance by injection.

The active agent release site may be at or near the site of a tumour in the subject. The active agent release site may also be at or near a tumour bed site, from which a tumour has been removed. Preferably the active agent release site is at a tumour or at a tumour bed.

The method of treatment or prevention of a disorder in a subject is typically performed in vivo, meaning that the active agent release site is typically in vivo. However, the source of the alternating magnetic field (e.g. the electromagnet or the source of radio waves) is typically located outside the body of the subject.

The method of treatment or prevention of a disorder in a subject is generally performed using a composition comprising the nanoparticle-polymer complex. Accordingly, step (a) of this method typically comprises providing a composition comprising the nanoparticle-polymer complex together with a pharmaceutically acceptable excipient. In some cases, the method of treatment may be performed using an article as described herein comprising the complex. The article may be a medical device, for instance a stent. In such cases the method may comprise a first step of inserting the article into the body of the subject, for instance a step of implanting a stent.

### Composition for Use

Also provided is a composition for use in methods of therapy as described in the preceding section. In particular, there is provided a nanoparticle-polymer complex for use in a method of treatment or prevention of a disorder in a subject, wherein the nanoparticle-polymer complex comprises an active agent and an iron oxide nanoparticle bound to a polymer capable of undergoing a phase change at a predetermined temperature, the method comprising
a) administering the nanoparticle-polymer complex to the subject; and
b) exposing the nanoparticle-polymer complex to an alternating magnetic field and thereby heating the iron oxide nanoparticle, causing the phase change to occur and active agent to be released.

Also provided is a composition for use in a method of treatment or prevention of a disorder in a subject, wherein the composition comprises a pharmaceutically acceptable excipient and an active agent and an iron oxide nanoparticle bound to a polymer capable of undergoing a phase change at a predetermined temperature, the method comprising
a) administering the composition to the subject; and
b) exposing the nanoparticle-polymer complex to an alternating magnetic field and thereby heating the iron oxide nanoparticle, causing the phase change to occur and active agent to be released.

### Non-Biological Methods

In some embodiments the method of releasing an active agent is performed in a non-biological system. That is, the method of releasing an active agent may not be performed in vivo. The method has many uses which are not biological applications; two exemplary methods are described below.

In one example, the method of releasing an active agent may be employed in the oil industry, e.g. in oil refining. The method is useful as it can be used to kill bacteria in oil pipes and hence keep the pipes clean. In such cases, the active agent is typically an antimicrobial agent, such as a bactericide.

For example, the method of releasing an active agent may comprise:
a) providing to an oil pipe a nanoparticle-polymer complex comprising an active agent and an iron oxide nanoparticle bound to a polymer capable of undergoing a phase change at a predetermined temperature; and
b) exposing the nanoparticle-polymer complex to an alternating magnetic field and thereby heating the iron oxide nanoparticle, causing the phase change to occur and active agent to be released;
preferably wherein the active agent is an antimicrobial agent.

The method is particularly useful in the oil pipes as oil pipes are difficult to access directly as they may contain environmentally undesirable fluid at high pressure. It is therefore undesirable to pierce the pipe and clean it. A method of cleaning the pipe which can be activated remotely is therefore highly desirable.

The complex can be removed from the oil pipe after the method is performed by a magnet inside the system or external to the system.

The method of releasing an active agent has similar applications in water-carrying systems, such as water pipes e.g. water treatment systems.

The pre-determined temperature for a complex according to the invention, when intended for use in a non-biological system, can vary. The pre-determined temperature may be in excess of 37° C., or in excess of 40° C., or in excess of 41° C. as described elsewhere herein. In some embodiments, the pre-determined temperature for complexes to be used in a non-biological system may be higher than is preferred for a biological system. For instance, where the nanoparticle-polymer complex is intended for use in a non-biological system, the pre-determined temperature may be 50° C. or more, such as 60° C. or more or 70° C. or more.

### Method of Releasing Two or More Active Agents

The method of releasing an active agent described herein may be a method of releasing more than one active agent. This can be achieved by employing a nanoparticle-polymer complex which comprises more than one active agent in the method.

Alternatively, this can be achieved by employing composition comprising two or more kinds of nanoparticle in the method. Such complexes and compositions have been described above.

The nanoparticle-polymer complex may comprise a first and a second active agent. Accordingly, in one aspect the method is a method a method of releasing two or more active agents, which method comprises:
a) providing a nanoparticle-polymer complex comprising a first active agent, a second active agent, and an iron oxide nanoparticle bound to a polymer capable of undergoing a phase change at a predetermined temperature; and
b) exposing the nanoparticle-polymer complex to an alternating magnetic field and thereby heating the iron oxide nanoparticle, causing the phase change to occur and the first and second active agents to be released.

The method can be more subtly tuned where the nanoparticle-polymer complex comprises two or more polymers. As explained above, the nanoparticle-polymer complex may comprise a first polymer associated with a first active agent (via a carrier or otherwise) and a second polymer associated with a second active agent (via a carrier or otherwise). The first polymer may be capable of undergoing a phase transition at a first pre-determined temperature and the second polymer may be capable of undergoing a phase transition at a second pre-determined temperature. The first pre-determined temperature may be different to the second pre-determined temperature.

Accordingly, in one aspect the method is a method a method of releasing two or more active agents, which method comprises:
a) providing a nanoparticle-polymer complex comprising (i) an iron oxide nanoparticle; (ii) a first active agent and a first polymer capable of undergoing a phase change at a first predetermined temperature bound to the iron oxide nanoparticle; and (iii) a second active agent and a second polymer capable of undergoing a phase change at a second predetermined temperature bound to the iron oxide nanoparticle; and
b) exposing the nanoparticle-polymer complex to an alternating magnetic field and thereby heating the iron oxide nanoparticle, causing the phase change to occur and the first and second active agents to be released.

Of course, if step (b) comprises heating the complex to a temperature which is above both pre-determined temperatures, both the first and second active agents will be released. However, if the complex is heated to a temperature between the first pre-determined temperature and the second pre-determined temperature, only the polymer which has the lower pre-determined temperature will undergo a phase change causing release of the associated active agent.

However, it may be complex to manufacture a nanoparticle comprising more than one active agent. Moreover, it may be desired to keep the active agents apart prior to release. More typically, therefore, the method involves heating a composition comprising a plurality of nanoparticle-polymer complexes as described herein.

For instance the method may be a method for releasing a first active agent and a second active agent, wherein the method comprises
a) providing a composition comprising:
(i) a first nanoparticle-polymer complex comprising a first iron oxide nanoparticle, the first active agent and a first polymer capable of undergoing a phase change at a first predetermined temperature bound to the first iron oxide nanoparticle; and
(ii) a second nanoparticle-polymer complex comprising a second iron oxide nanoparticle, the second active agent and a second polymer capable of undergoing a phase change at a second predetermined temperature, bound to the second iron oxide nanoparticle; and
b) exposing the composition to an alternating magnetic field.

Release of the first active agent alone or together with the second active agent can be achieved by heating the composition above one or both of the pre-determined temperatures.

### Use of Complex to Release an Active Agent

The invention also provides the use of a nanoparticle-polymer complex comprising an active agent, as described herein, to release an active agent. Typically said use is use in a method of releasing an active agent as described herein. For instance, the use may be a use in a method of therapy or in a non-biological method.

### EXAMPLES

1. Materials

All materials were purchased from Sigma Aldrich unless specified otherwise.

1. Synthesis and Experimental Methods.

Cell culture: The biological effect of citral and various nanoparticle-polymer complexes were tested in vitro on Rhabdomyosarcoma (RMS) immortalized cancer cell lines obtained from the American Type Culture Collection (ATCC; Manassas, Va., USA). The three cell lines tested were RD (ATCC code CCL-136), RH30 (ATCC code CRL-7763) and the U87 cell line. U87 is a human primary glioblastoma cell line that is commonly used in brain cancer research (ATCC code HTB-14). Cells were grown in growth medium (Dulbecco's modified Eagle's medium [Sigma-Aldrich]) supplemented with 10% fetal calf serum (Sigma-Aldrich), 2 mM L-glutamine (Sigma-Aldrich), 100 U/ml penicillin (Sigma-Aldrich), and 0.1 mg/ml streptomycin (Sigma-Aldrich) and then incubated at 37° C. in a 5% CO2 atmosphere. Cells were passaged every 3-4 days. Cells were seeded in 96-well plates at 1×10⁴ cells/well in growth media and left overnight in the incubator for the cells to adhere.

Cell viability experiments: Cells were seeded in 96-well plates at 1×10⁴ cells/well in growth media and left overnight in the incubator for the cells to adhere. The following day cells were treated for 24 h with concentrations between 1 and 1000 μM citral. After treatment, the media on the cells was removed and the cells were washed in PBS twice. Cells were fixed in 100 μl of 1% (v/v) glutaraldehyde (aq; Sigma-Aldrich) for 30 min and stained with 100 μl of 0.5% (w/v) crystal violet solution (aq; Sigma-Aldrich) for at least 1 h. The plate was washed with water and dried overnight and cells were solubilized using 150 μl of solubilizing solution (1% [w/v] sodium dodecyl sulfate [Fisher Scientific]) and 10% (v/v) acetic acid (Sigma-Aldrich)]. The absorbance of the solution was measured at 590 nm using a Tecan Infinite f200 microplate reader. Samples were blank corrected and expressed as a percentage of the control cell viability. Experiments were performed in triplicates and repeated on three separate occasions.

Vitamin C and Vitamin K3 synergistic experiments: After obtaining cell viability data on the differing combinations of vitamin C and vitamin K3 bliss additivity calculations were used to assess the most efficient synergistic combinations. The ideal being the greatest cell death in comparison to the cell death caused in the absence of the other molecule in solution.

Synthesis of lauric acid stabilised iron oxide nanoparticles (LA-ION): For preparation of LA-ION, 0.56 g of Iron(II) chloride and 2.35 g of Iron(III) Chloride hexaqua were dissolved in 25 ml of ddH₂O under a Nitrogen atmosphere. Next, the solution was gradually heated to 80° C., during heating 0.1 g of lauric acid in 5 ml acetone was added and immediately following this 5 ml of 28% ammonium hydroxide was added. Once the solution had reached 80° C., 5 aliquots of 0.2 g of lauric acid in 5 ml acetone were added over 5 minutes. The solution was allowed to cool and precipitated out in 50 ml of ethanol and 50 ml of acetone, the product removed by magnetic decantation.

Anchoring DDMAT to iron oxide nanoparticles via ligand substitution: To enable ligand exchange, 50 mg of freshly synthesised lauric acid stabilised iron oxide nanoparticles were dispersed in 20 ml of 1,2-dichlorobenzene. 1 g of DDMAT was then added and the solution stirred at 80° C. for 24 h. The DDMAT stabilised nanoparticles (DD-ION) were precipitated in methanol and washed in several cycles of dispersion in THF and re-precipitation in methanol.

Outright synthesis of DDMAT stabilised iron oxide nanoparticles: Particles were prepared as in the lauric acid stabilised particles mentioned above with DDMAT as the stabilizing agent in place of lauric acid. The same masses and molar ratios were used. Everything in the reaction was kept consistent with the synthesis of LA-ION as was the precipitation and washing procedure.

RAFT polymerisation of unbound polymers: For the synthesis of the free polymer, 25 mg of DDMAT was dissolved in 10 ml THF under a nitrogen atmosphere. Monomer was added to final a concentration of 10 mM and 1.17 mg AIBN was added, still under a nitrogen atmosphere. The reaction flask was then degassed by three freeze-thaw pump cycles and heated to 65° C. and stirred for 24 h. After 48 h the solution was diluted with THF and precipitated in diethyl ether, centrifugation and washed three times.

RAFT polymerisation of iron oxide nanoparticle bound polymers: Polymers were synthesised as above in the aforementioned PNIPAM synthesis method, with 200 mg of DDMAT stabilised iron oxide particles being used in place of 25 mg DDMAT.

RAFT polymerisation of bound and unbound copolymers: Synthesis of copolymers was done by the same method of bound and unbound monomers respectively but with the desired monomers being added in their desired ratios together. For the synthesis of block copolymers, the synthesis was undertaken in an anaerobic chamber, after given time intervals co-monomers were added and the reaction allowed to continue. Once the reaction was deemed completed the product was extracted in the same method as described above.

Synthesis of thermosensitive polymer bound to iron oxide nanoparticles: Lauric acid-stabilised iron oxide nanoparticles were synthesised, and DDMAT was then attached by ligand exchange as described above. 150 mg of these DDMAT-stabilised iron oxide nanoparticles were dissolved in 5 ml DMF under a nitrogen atmosphere. Monomer(s) were added in various ratios to a total molar concentration of 10 mM and 1.17 mg AIBN was added, still under a nitrogen atmosphere. The reaction flask was then degassed by three freeze-thaw pump cycles and heated to 65° C. and stirred for 24 h. After the desired reaction times the solution was diluted with THF and precipitated in diethyl ether, and the product was removed with a magnet and washed three times. In some cases, block copolymers were created as follows. Synthesis was undertaken in an anaerobic chamber and initially started with only methacrolein present. After one week the co-monomers NIPAM and acrylamide, were added and the reaction was allowed to continue for a further week. Once the reaction was deemed completed the product was removed with a magnet and washed three times.

Exchange of bound ligands/polymer form iron oxide nanoparticles for analysis: In order to analyse grafted polymers, 50 mg of polymer grafted iron oxide nanoparticles were dissolved in 10 ml of glacial acetic acid and 10 ml of chloroform. The solution was stirred at room temperature for 24 h. The precipitants were removed by centrifugation and magnetic decantation and discarded, the clear solution remaining was removed and evaporated to dryness. It was then washed with THF and dried once more for analysis.

Recrystallisation of Acrylic acid: Purification of Acrylic acid was done by dissolving 30 ml of glacial acrylic acid containing hydroquinone as a stabiliser in 50 ml of diethyl ether. The solution was then cooled to 0° C. until Acrylic acid crystals were visible and crystal formation had stopped. The product was separated by vacuum filtration and washed with cold diethyl ether. Samples were then tested for stabiliser presence by HPLC.

LCST measurement of polymers: Samples of PNIPAM and PN-ION were made up to 10 mg/ml in clear colourless glass vials, these were then placed in water above a hotplate and heated. Reading off a mercury thermometer placed in the water, the LCST for each sample was observed. Three measurements were taken for each sample and an average temperature taken. For more accurate measurements the 10 mg/ml polymer samples were heated to 90° C. and placed inside a UV-VIS spectrometer. The temperature was monitored via the use of thermo couples and the LCST determined by the temperature at which the solution hit an absolute minimum adsorption at 800 nm.

Binding paclitaxel to bovine serum albumin: Paclitaxel (1.2 mg) was dissolved in 100 uL pure ethanol. This was combined with 9.9 ml pure H₂O containing 6 mg BSA and the solution was stirred at 21° C. for 2 hours. The BSA-PAC complex was removed by precipitating out with ethanol and centrifugation at 13,000 g for 5 minutes, the precipitate was then washed thrice with ethanol. Paclitaxel content was measured by degrading the BSA in trypsin for 30 minutes at 37.5° C. and quantified by HPLC measurement and peak integration.

Binding Vinblastine to bovine serum albumin: Vinblastine was bound to BSA in the synonymous method as Paclitaxel, with the same mass of Vinblastine used in place of paclitaxel.

Binding Paracetamol to bovine serum albumin: Paracetamol was bound to BSA in the synonymous method as Paclitaxel, with the same mass of paracetamol used in place of paclitaxel.

Immobilising bovine serum albumin on polymer: The equilibrium solution was formed with 8 mg of copolymer and 6 mg of BSA dissolved in 5 ml PBS. The solution was cooled to 4° C. and stirred for 24 hours. After this, solutions were raised above the LCST of the polymer and precipitants were removed by centrifugation at 10,000 g and washed three times with PBS. To measure the immobilised BSA the polymer was stirred for 5 minutes below the LCST in 1 ml PBS, then stirred for 5 minutes above the LCST and finally precipitated out. The supernatant was tested for protein content by a standard Bradford protein assay. This process was repeated three times to ensure maximum detection of immobilised protein.

Immobilising drug-BSA complex on nanoparticle-polymer complex: an equilibrium solution was formed with 16 mg of the nanoparticle-polymer complex (the polymer comprising methacrolein, acrylamide and NIPAM monomer units) and 6 mg of the BSA-drug complex dissolved in 5 ml PBS. The drug in question was paclitaxel, vinblastine or paracetamol as described above. The solution was cooled to 4° C. and stirred for 24 hours. After this, solutions were raised above the LCST of the polymer and precipitants were removed by centrifugation at 10,000 g and washed three times with PBS before lyophilisation.

Inductive heating equipment setup: Inductive heating was carried out using a 1000 W ZVS Low Voltage Induction Heating Board with a Module Tesla Coil and a Flyback Driver Heater. The working coil consisted of a copper Tube wound with 10 coils and a 1-inch coil diameter. The supply to the circuit board was a currant and voltage variable laboratory power supply system.

Inductive heating of iron oxide nanoparticles: Varying weights of ION were dissolved in 5 ml pure water in a glass vial and sonicated before being cooled to 4° C. prior to heating. The glass vial was insulated on every side by polystyrene foam to minimize heat input from the coil. The voltage was varied by the power supply and the temperature of the solution was measured every minute using a non-mercury or non-conductive thermometer.

3. Analysis Methods:

Centrifugal sedimentation: The hydrodynamic diameter of the nanoparticles was measured using a CPS disc centrifuge (DC 18000; CPS Instruments Europe, Oosterhout, the Netherlands). The machine was operated at 24,000 rpm, and sucrose density gradient ranging from 8% to 24% (w/v) was built up by injecting decreasing concentrations of sucrose into the centrifuge. Samples were prepared by resuspending the nanoparticles in double distilled water, sonicating with an ultrasonic probe (sonic Vibra-Cell) for 5 min (104 W [80%] 5 sec on/5 sec off) and filtering with a 0.2 μm filter (33 mm cellulose acetate syringe filter, Anachem). Samples were calibrated against particles of a known diameter (polyvinyl chloride, 0.377 μm, CPS Instruments Europe).

Zeta surface potential measurements: The zeta potential of the nanoparticles was measured using a Malvern Zetasizer Nano. Samples were prepared at a concentration of 0.2-0.3 mg ml-1 in PBS.

Gas chromatography: Detection of DDMAT by gas chromatography was performed using a Shimadzu GC 2010. The samples were separated with a ZB-FFAP column (Zebron, Phenomenex) and coupled to an FID detector. An optimized protocol to give a clear unmasked signal for DDMAT was as follows; temperature maintained at 100° C. for 5 min, followed by ramping to 240° C. at a rate of 50° C. per minute, and finally held at 240° C. for 5 minutes. Total run time was 28 minutes and 30 s. This temperature was then maintained for 4 min to ensure complete flushing of the column. Samples were dissolved in HPLC grade ethanol before analysis.

High performance liquid chromatography: Samples of Paclitaxel were analysed on an Agilent 1120 compact LC. The column used for stationary phase was a ZORBAX Eclipse 18C β-karoteen column (4.6×150 mm 5 micron), detection was run at 228 nm with a deuterium lamp. The mobile phase for the separation of paclitaxel consisted of 58% acetonitrile and 42% (HPLC grade) water. A Paclitaxel peak was detected at 5.88 minutes and each run had a finishing time of 10 minutes.

Transmission electron microscopy: TEM images were taken of nanoparticles using a JEOL JEM-2100Plus microscope, a multipurpose transmission electron microscope combining JEM-2100 optical system with an advanced control system for enhanced ease of operation. 5 mg samples were dissolved in ethanol or tetrahydrofuran depending on solubility, for dispersion on the imaging plate. The samples were then imaged under a 200 Kv beam.

Scanning electron microscopy: Scanning electron microscopy (SEM) was used to characterize the wafers using a Carl Zeiss LS15 VP machine at 15 kV. SEM specimens were sputter coated with a 5 nm layer of Au—Pd.

Ultraviolet-visible light spectroscopy: A Shimadzu UV-1800 UV-VIS spectrometer was used alongside Carry WinUV thermocoupling software that was used for thermodynamic data processing and analysis. Samples of PNIPAM and PN-ION were prepared in aqueous solution at 30 mg/ml and sonicated prior to analysis. Absorbance was measured at 800 nm as this was the wavelength of maximum absorbance difference between PNIPAM in its hydrophilic state to its hydrophobic state. Temperature and absorbance readings began at 40° C. and samples were cooled to a temperature of 4° C.

Fourier transform infrared spectroscopy: An Excalibur series FTS 3500 instrument was used and is equipped with the following: A diamond ATR head, a DRIFT attachment for powders, a gas cell, a transmission cell and an improved resolution MCT detector. The Frontier FT-IR is a bench top instrument used to analyse samples such as hydrocarbons, oils and pharmaceutical samples. It was used for the determination of surface functional groups on a sample.

Nuclear magnetic resonance spectroscopy: For both C¹³ and H¹ 500 MHz Bruker Avance were used for a wide range of multinuclear NMR experiments and variable temperature spectroscopy. MestReNova version 11.0.01 software was used to analyse the spectrums. The free polymers and polymers exchanged from grafting on nanoparticles were dispersed in deuterium oxide or deuterated chloroform depending on solubility, at a concentration of 30 mg/ml.

Differential scanning calorimetry: A Perkin Elmer Diamond instrument with a helium and nitrogen gas supply was used to obtain thermal properties of polymers. 8 mg polymer samples were prepared in crimped sealed aluminium pans. The scan rate set at 100 cycles per second and the temperature range was set from 0° C. to 250° C. with a heating rate of 100° C. per minute. Results were presented in an endo up format.

Statistical analysis: Significance was tested using a two-tailed t-test comparing treated and untreated cells (not significant p≥0.05, *p≤0.05, **p≤0.01, ***p≤0.005).

4. Synthesis and Characterisation of Iron Oxide Nanoparticles

Fe₃O₄ super paramagnetic particles were synthesised and subsequently analysed by several methods. Larger Fe₃O₄ particles were produced by using smaller particles as nucleation seeds. To confirm the identity, size, shape and morphology of particles; TEM, FT-IR, Zeta potential and centrifugal sedimentation were used. The black nature of the product confirmed the identity of Fe₃O₄ particles, in contrast to the white nature of Fe₂O₃ particles. A yield of 77-86% was typically obtained from the synthesis method after three cycles of washing in a 50:50 mixture of acetone and ethanol and subsequent magnetic decantation.

Fe₃O₄ super paramagnetic particles sterically stabilised with lauric acid ligands (LA-ION) were synthesised and used as a base to produce the nanoparticle complex. Lauric acid was used as a stabilising agent, due to its ready availability, ease of handling and existing literature methods. Seeding was carried out by using 200 mg of lauric acid stabilised particles (referred to as V1) in a 1 g synthesis of larger particles. A second round of seeding was run on the larger particles (referred to as V2) to again increase their size, with a yield of 1 g from 200 mg seed. The size and zeta potential (surface charge) of the nanoparticles produced by these methods is given in Table 1 below.

TABLE 1
LA-ION physiochemical characteristics
including size, and surface charge.

| | | Zeta |
|---|---|---|
| Fe₃O₄ particles | Average diameter (nm) | potential (mV) |
| Non seeded (V1) | 7.13 (TEM) SD = +/−2.1 | +0.381 |
| Seeded (1^{st} seed) (V2) | 18.5 (CPS) SD = +/−6.8 | −1.40 |
| Seeded (2^{nd} seed) (V3) | 32 (Disc cent) SD = +/−18.6 | +1.43 |

Zeta potential measurements confirmed the neutrality of the surface of the particles, indicating a good coverage of ligands and stability. The surface potential showed negligible change upon synthesising larger particles, promising for consistent behaviour in future experiments.

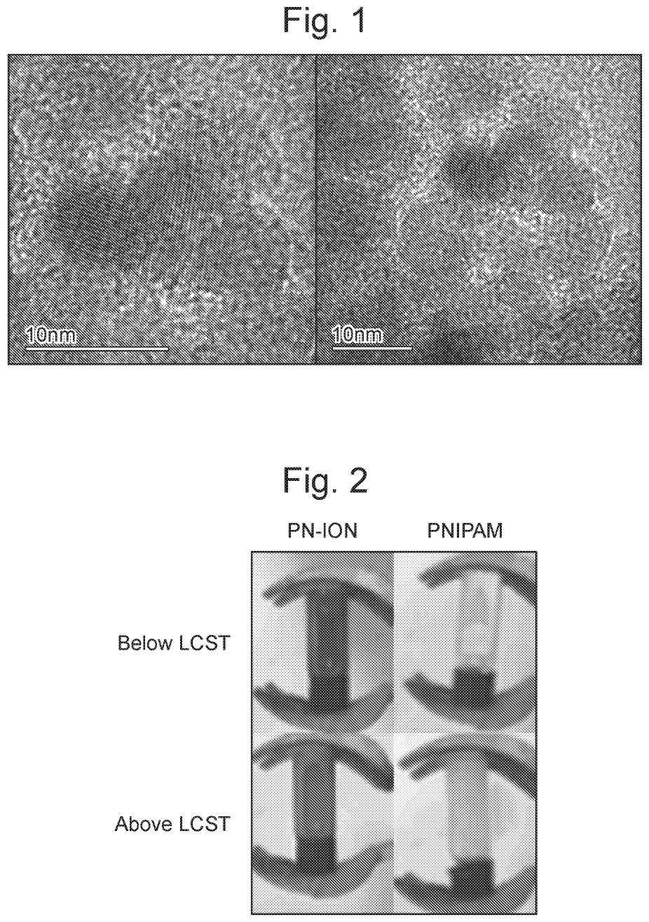

TEM imaging allowed accurate size determination of the particles to be carried out. 30 particles were measured (using Gatan 3 imaging software) for diameter and an average size and standard deviation calculated. From TEM images (shown in FIG. 1) the average size was determined to be 7.13 nm, with a SD of 2.1 nm. The atomic spacing can be seen via the repeating “rows” in each crystal, representing the space between the iron atoms in the structure, occupying the holes in the fcc oxygen crystal lattice. The spacing was calculated at 2.7 Å, which is within the range expected for a magnetite crystal. The morphology of the particles was also investigated in these images, particles were spherical in all dimensions with very good homogeneity. Importantly, the particles showed a single crystal nature. This is useful in ensuring the superparamagnetic properties of the particles and that a single magnetic domain is present, which assists efficient inductive heating.

As expected, the diameter increases with seeding however. Although the larger particles are closer to the ideal size for cell uptake (around 50 nm), the extra steps in synthesis and the potential decrease in inductive heating effect means the 7 nm (unseeded particles) were used as the basis for particles in the following experiments.

5. Attachment of Anchoring Monomer to Iron Oxide Nanoparticle

Lauric acid Fe₃O₄ super paramagnetic particles synthesised were used as the reagent in a ligand exchange synthesis to produce DDMAT stabilised particles (DD-ION). Particles were washed in a similar fashion to that of the synthesis procedure and analysed in the same methods. FT-IR analysis of the product confirmed the presence of DDMAT stabilised particles. This is referred to as the “exchange synthesis” method. The yield of this synthesis was 94%, based on the recovery of nanoparticle mass.

In addition to adding the anchoring monomer (DDMAT) to the iron oxide nanoparticle after ligand exchange, iron oxide particles carrying the anchoring monomer were also generated using DDMAT as the stabilising ligand in the synthesis of the particles. This is referred to as the “outright synthesis” method. This reduced the amount of DDMAT needed. To measure the efficiency of uptake in this new synthesis method in comparison to the previously-described method, the total grafted DDMAT from 100 mg particles were removed by a further ligand exchange and tested for quantitative measure by GC analysis. After a ligand exchange reaction and subsequent purification of the cleaved groups from the surface, the concentration was determined against known samples in the GC. A calibration curve was used to gain an accurate relationship between peak area and DDMAT concentration and samples were run against an internal standard to ensure validity. DDMAT content was measured as a concentration and converted into total mass. The total weight was compared to the total weight of the nanoparticles and presented as a percentage weight of the entire complex. The results are presented in Table 2, below.

TABLE 2
Quantification of loading of DDMAT on DD-ION by
the different investigated synthesis methods.

| | mg DDMAT/mg | % weight of DDMAT in |
|---|---|---|
| Synthesis Method | particle | particle |
| EXCHANGE SYNTHESIS | 0.0577 | 5.77 |
| OUTRIGHT SYNTHESIS | 0.1433 | 14 |

The total loading achieved by the outright synthesis was over twice that of the exchange synthesis method presented in literature.

There was no difference in the FT-IR spectra and negligible differences in the size, shape and morphology of the particles between the difference synthesis methods.

Loading of the anchoring monomer (e.g. DDMAT) onto the iron oxide nanoparticles is important when performing free radical polymerisation on the surface of these particles. The content of DDMAT (or another anchoring monomer comprising a polymerisable moiety) in the polymerisation reaction must be in excess of the number of propagating chains for proper polymer control to be established. Naturally the greater content of anchoring monomer per weight of particle will provide a more controlled polymerisation and allow for increased chances of the polymer remaining grafted on the surface of the particles.

6. Polymer Synthesis (1)

As the polymer produced is formed from a multiple monomers, it may be referred to herein as a copolymer.

The polymers were analysed by methods described above including FT-IR, H¹ and C¹³ NMR, DSC, UV-VIS and SEM.

The LCST of the polymers was determined by UV-VIS spectroscopy combined with thermocoupling equipment. Specifically, following washing and isolation of the polymer, UV-VIS spectroscopy was used to determine the LCST by observing a change in absorption at 800 nm upon the phase change. The UV-VIS absorption was run as a function of time and run concurrently with thermocouples. The absorption and temperature data alignment were made possible by the thermocoupling software and an absorption-temperature graph created. This method was used as the principle method of the determination of the LCST of polymers described hereafter.

NIPAM was selected as the phase change monomer.

Acrolein was selected as the binding monomer as it has been used before to immobilise proteins and is a relatively well studied compound. It can immobilise proteins such as BSA by interaction of its aldehyde group with functional groups within the complex structure of the protein. Most notable of these interactions is the imine (C═N) bond formation under mild conditions between its carbonyl group and a primary amine group in the protein, forming a Schiff base linkage. Further, its simplicity makes polymerisation predictable and it behaves well in most polymerisation models.

However, the hydrophobic nature of the acrolein monomer results in a smaller negative enthalpy from the hydrogen bonding of the polymer with water. This decreases the LCST of a copolymer that contains acrolein. Since the desired phase change temperature is above body temperature, hydrophilic monomers must be added to counterbalance this effect.

Accordingly, the hydrophilic monomer acrylic acid (AA) was selected as the LCST-adjusting monomer. It was not initially known how the LCST of the resulting copolymer would be affected by inclusion of these binding monomers and LCST-adjusting monomers in a copolymer with the phase change monomer.

A copolymerisation was performed, including all three constituent monomers. 25 mg of the anchoring monomer DDMAT was also included in the monomer mixture. The reaction scheme is shown below.

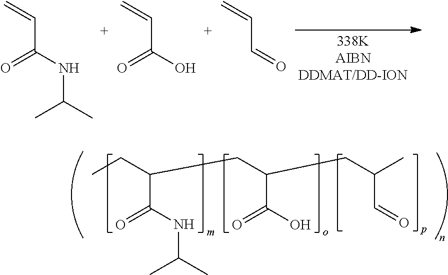

The relative molar ratios of the monomers were altered to change the LCST of the terpolymer. A number of variants were produced: Type 1 (T1), Type 2 (T2) etc. The structure of each type of terpolymer was determined again by FT-IR and NMR. Since the differing types vary only in molar ratio, it was found that the fine structure of the polymer was be very similar for each polymer type, excluding those where a constituent monomer has a content of 0. The presence of a polymer was first verified by the appearance of a white precipitate in the extraction procedure.

In each case it was concluded that the polymerisation of the terpolymer was successful, as confirmed by the FT-IR and NMR data.

LCST tests were carried out as described above and the results are shown in Table 3, below. As expected, with an increase in acrolein molar ratio the LCST of the terpolymer decreased. However, it was noticed with increasing acrylic acid content, the LCST decreased.

TABLE 3
Variation of LCST of terpolymer with molar
ratios of the constitute monomers.

| NIPAM % | Acrolein % | Acrylic acid % | LCST/° C. |
|---|---|---|---|
| 100 | 0 | 0 | 32 |
| 50 | 50 | 0 | 14 |
| 50 | 0 | 50 | 24 |
| 50 | 25 | 25 | 19 |
| 45 | 25 | 30 | 18 |
| 40 | 25 | 35 | 17 |
| 40 | 20 | 40 | 23 |
| 40 | 25 | 35 | 20 |
| 93.3 | 0 | 6.7 | 30 |
| 80 | 0 | 20 | 28 |

The LCST becomes higher as NIPAM is copolymerized with hydrophilic acrylic acid monomer, but it decreases above 10% acrylic acid molar ratio. This is due to the hydrogen bond between —COOH in acrylic acid and the amide groups in NIPAM forming a stronger hydrogen bond than the one between H₂O and the amide groups. Therefore, there is less hydrogen bonding between H₂O and the amide groups to break down upon the phase change, which causes the LCST to decrease. On the other hand, when the molar content of acrylic acid is much more than NIPAM, the hydrogen bond between H₂O and acrylic acid becomes dominant. The copolymer trends to dissolve and LCST increases.

The effect seems to continue when 20% molarity of acrolein is used.

7. Polymer Synthesis (2)

A second polymer synthesis was performed, and is illustrated by the following mechanism.

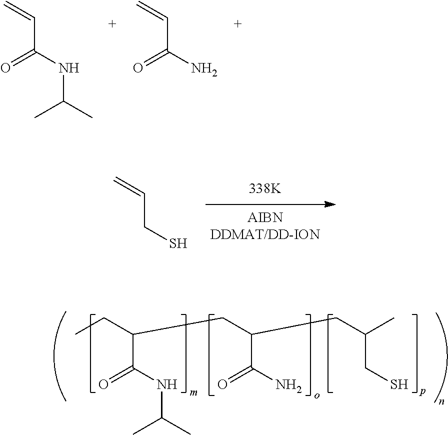

NIPAM was selected as the phase change monomer.

Allyl mercaptan (allyl thiol) was selected as the binding monomer. It has been used before to immobilise proteins and is a relatively well studied compound. It can immobilise proteins such as BSA by interaction of its thiol group with functional groups within the complex structure of the protein. Most notable of these interactions is disulphide bond formation with thiol groups present in the protein itself. Its simplicity makes polymerisation predictable and it behaves well in most polymerisation models. The hydrophobic nature of the allyl thiol monomer results in a smaller negative enthalpy from the hydrogen bonding of the polymer with water. This decreases the LCST of a copolymer that contains the thiol group, and since the desired phase change temperature is above body temperature, hydrophilic monomers must be added to counterbalance this effect.

Accordingly, acrylamide was added as the LCST-adjusting monomer. It was not initially known how the LCST of the resulting copolymer would be affected by inclusion of these binding monomers and LCST-adjusting monomers in a copolymer with the phase change monomer.

A copolymerisation was performed, including all three constituent monomers. 25 mg of the anchoring monomer DDMAT was also included in the monomer mixture. A number of variants were produced: Type 1 (T1), Type 2 (T2) etc. The structure of each type of copolymer was determined again by FT-IR and NMR. Since the differing types vary only in molar ratio, it was found that the fine structure of the polymer was be very similar for each polymer type, excluding those where a constituent monomer has a content of 0.

Synthesis of the copolymer resulted in the formation of a light brown powder. Upon being left in air a change in colour of the light brown powder to a dark brown almost black powder, as a result of the oxidation reactions of thiol groups present in the polymer to disulphide bridges.

LCST tests were carried out as described above and the results are shown in Table 4, below. As expected, with an increase in acrylamide content, the LCST of the terpolymer increased. This was observed even when allyl thiol was added to the terpolymer.

TABLE 4
Variation of LCST of terpolymer with molar
ratios of the constitute monomers

| | Allyl | | |
|---|---|---|---|
| NIPAM | Thiol | Acrylamide | LCST (° C.) |
| 95 | / | 5 | 34 |
| 80 | / | 20 | 41 |
| 50 | / | 50 | 84 |
| 60 | 20 | 20 | 36 |
| 55 | 20 | 25 | 41 |
| 50 | 20 | 30 | 49 |

One of these polymers exhibited the particularly desirable desired LCST of 41° C. therefore these monomer ratios were taken forward for synthesis of the polymer at the surface of the iron oxide nanoparticles in Section 9. This is referred to as polymer type 3 (T3).

8. Synthesis of PNIPAM Bound to Iron Oxide Nanoparticle

The most convenient method of providing a polymer bound to the iron oxide nanoparticle is to synthesise the polymer directly from the surface of the iron oxide nanoparticle. This can be accomplished by using the chain transfer agent DDMAT as a stabilising ligand. During the RAFT polymerisation reaction polymer chains remain bonded by the trithiol group contained within DDMAT and hence remain bound to the nanoparticle.

Polymerisation reactions were performed in the same manner as free polymer reactions with the exception that the 25 mg DDMAT component was replaced with 150 mg DD-ION (i.e. iron oxide nanoparticles bound to DDMAT anchoring ligands). The anchored polymer yield resulted as 0.989 g of product produced from 1.14 g of monomer, however 100 mg of the product can be assigned to the weight of iron oxide nanoparticles in the final weight. Accounting for this gives a final yield of 77.9%, giving rise to idea that the binding of DDMAT to a nanoparticle surface has little effect on yield of polymerisation.

FT-IR analysis indicated that copolymerisation was successful.

The stability of the complexes comprising the polymer bound to an iron oxide nanoparticle in aqueous solution was tested with the aim of determining whether anchored polymers are hydrolysed into free solution when solvated. To examine this, 50 mg of the complexes were dissolved in 10 ml of water and compared with another 50 mg dissolved in the equivalent volume of methanol. These solutions were stirred at room temperature for 24 hours, evaporated to dryness and washed with diethyl ether before re-precipitation and magnetic decantation. The change in weight was determined and found to be 4 mg and 6 mg for the water and methanol solutions respectively. This shows only 8% and 12% of the sample weight was lost, likely due to the imperfect yield of the extraction method. Thus, the complexes had good stability in aqueous solution.

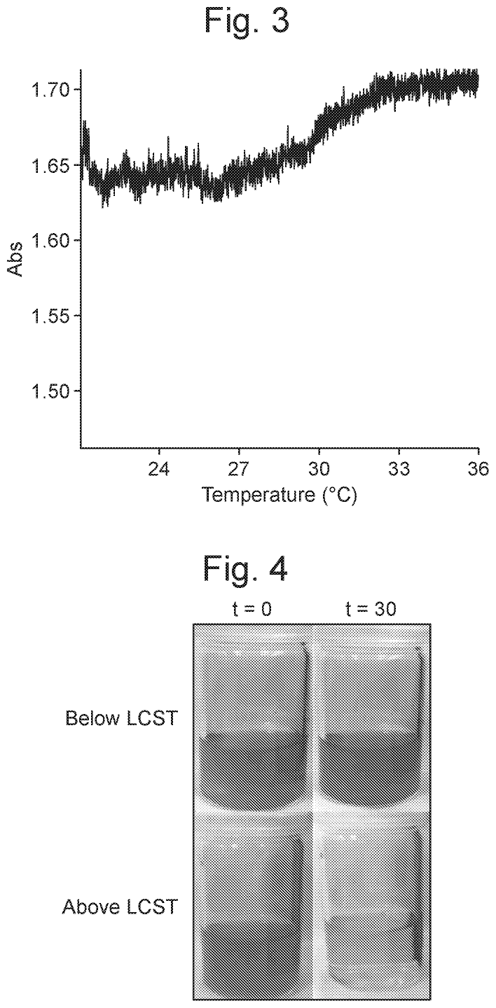

LCST measurements were performed on the complex. Because the complex formed as above is dark brown in solution (see FIG. 2), it is difficult to visualise the LCST using UV-VIS spectroscopy as the change in absorption due to the phase change is small (for instance compared to the change for a solution of PNIPAM which changes from colourless to white). However, after data processing and creation of the absorption vs temperature graphs the LCST for the complex as above was obtained, and is shown in FIG. 3.

Interestingly the LCST of the complex was observed at 26.2° C., which can easily be explained by the hydrophobic nature of the surface of Fe₃O₄ reducing the negative enthalpy contribution for dissolution of the whole complex in water. Hence, a lower temperature is required to cause the phase change of the polymer chains compared to the LCST of PNIPAM in solution.

9. Synthesis of Copolymer Bound to Iron Oxide Nanoparticle

A complex comprising polymer type 3 (T3) bound to the surface of the iron oxide nanoparticle was then synthesised (T3-ION). This complex served as the first nanoparticle-polymer complex to test protein immobilisation in the subsequent examples. It was synthesized akin to the protocol of example 8 above and separated and purified via magnetic decantation. All complexes were magnetic and soluble in water at low temperatures. A yield of 71% was observed.

When dispersed in water at room temperature the complexes, or particles, were stable for longer than 24 h. Even after settling on the bottom of a vial, particles were easily resolubilized with the slightest agitation. When placed next to a magnet, particles remained in solution while below the LCST. However, when heated to above the LCST the particles are drawn to a magnet immediately. Within 30 s of an applied magnetic field the particles accumulate in the direction of the field, leaving only negligible particles in solution as a result of Brownian motion. The effect of a magnetic field on these complexes can be seen in FIG. 4.

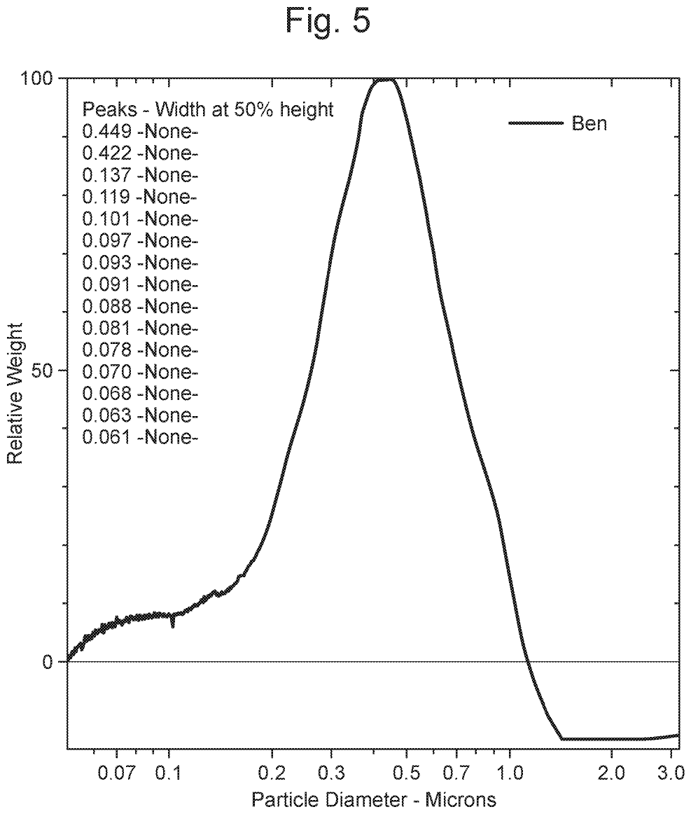

Interestingly when placed in solution at room temperature a peak hydrodynamic diameter of 450 nm was observed for the complex, which is much larger than the size of the iron oxide nanoparticle (which is −7 nm). This is the result of a polymer shell forming a layer around the particles. It was considered that the large size was unlikely to be due to aggregation of the complexes in solution as the signal showed a single well-defined peak, unlike the broad peak that would be expected as a result of aggregation. Data in this regard can be seen in FIG. 5.

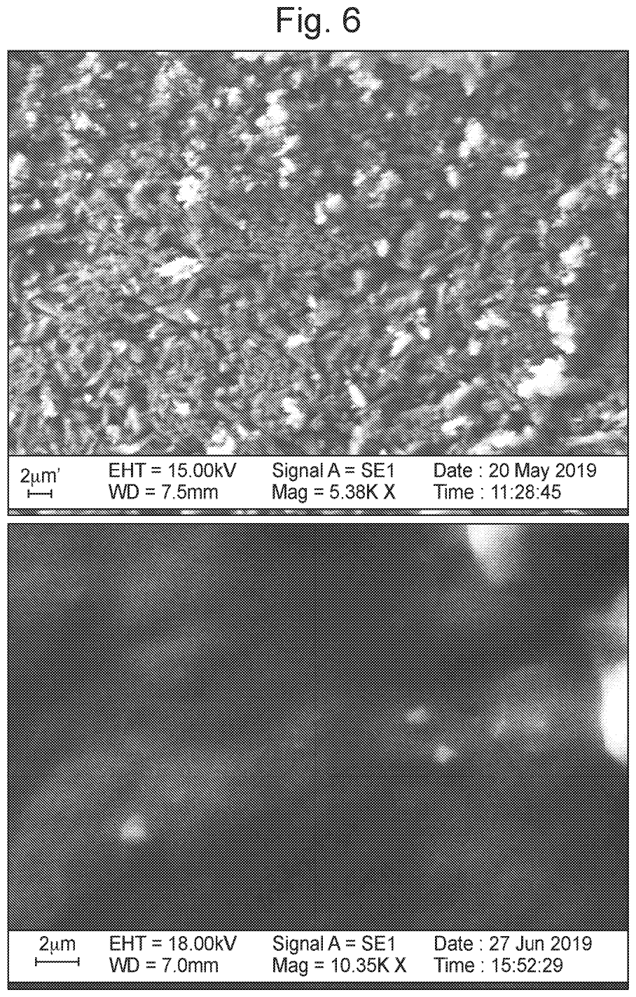

To confirm this theory SEM images were taken of the complex. Particulates were observed and a mean diameter of 430 nm (SD 74) were observed, consistent with the centrifugal sedimentation analysis peak shown in FIG. 5. The morphology of the particulates were spherical in nature although were often distorted. These observations support the idea of the synthesised polymer forming a shell around the nanoparticle core while in a solid dry state. The discrepancies in size and shape are expected as a result of the changes in the molecular weight of polymer chains formed and the number of anchored polymer chains to each particle. Two SEM images are shown in FIG. 6.

10. Inductive Heating of the Complex

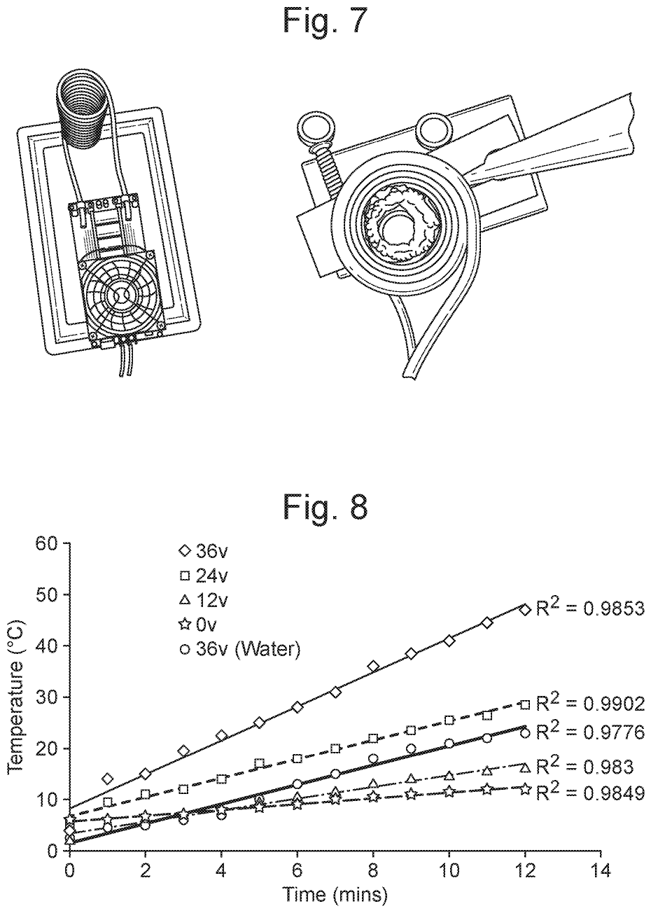

Induction heating was carried out in the lab after the assembly of the necessary equipment, a 112 kHz AC current was run through a 14-turn copper coil. The samples were placed within a polystyrene insulating container to prevent heat input from external sources. The samples were samples of the complex produced in example 8. Images of the setup including the electromagnet coil surrounding a sample vial are shown in FIG. 7.

The complex was dissolved in 5 ml of water to make a 20 mg/ml solution, this was sonicated to ensure dispersion and was then cooled to 4° C. Before being placed in the coil the vial was insulated with Styrofoam to minimize external heat input from the coil itself, a thermometer was placed inside the vial to record changes and the temperature was read every minute. The heating was tested for 12 minutes (to prevent overheating of circuit board) at 3 different set running voltages. Temperature couldn't be measured via a digital thermometer or thermocoupling equipment due to the metal within each device also becoming inductively heated in the field of the experiment. Therefore, temperature was measured manually by a non-metallic laboratory thermometer, placed directly in solution. Results of temperature as a function of time at different applied voltages are shown in FIG. 8.

The heating of the nanoparticles showed a very linear trend with time for all 3 running voltages. The increment in temperature was 3.5° C. min⁻¹, 1.92° C. min⁻¹, and 1.1° C. min⁻¹ for 36, 24 and 12v respectively. The temperature increase was very consistent across the voltages and throughout the heating, this is expected since the superparamagnetism of the particles should not change at these temperature ranges. The curie temperature of magnetite is 858 K and therefore the magnetic domain environment of the particles remains consistent at these temperatures. The phase change of the grafted polymer seems to have had no effect on the heating increment, the LCST that occurs at 26° C. for these complexes shows no change with the heating rate.

Thermal parameters of the heating processes shown in FIG. 8 are shown in Table 5, below.

TABLE 5
Thermal parameters of the inductive
heating of the polymer complex.

| | Starting | | |
|---|---|---|---|
| Applied | Temperature | End Temperature | Temperature change |
| voltage | (° C.) | (° C.) | per min (° C.) |
| 36 v | 4 | 47 | 3.58 |
| 24 v | 5 | 28.5 | 1.95 |
| 12 v | 2 | 16 | 1.17 |
| 0 v | 6 | 12 | 0.5 |
| 36 v (Water) | 2.5 | 23 | 1.71 |

A solution of just 2% (w/w) of the complex can heat 5 ml of water at a rate of almost 4° C. min⁻¹. This is a good demonstration of the massive heating capabilities of the synthesised nanoparticles. This very low concentration shows promising ability to cause the phase change of the complex and release of drugs, with minimal radio wave irradiation. It is promising data when looking to cause a temperature change in tumour tissue with a minimal concentration of particles.

While heating the PN-ION via inductive heating it was noticed the grafted polymer underwent its LCST phase transition. This was observed visually; before heating, the solution was a cloudy brown colour whereas after heating to above the LCST, the sample had turned opaque and white. This showed that the complexes were heated above their LCST and the phase change was triggered. As with conventional heating methods, upon the phase change the complexes are readily attracted to a magnet, whereas below the LCST they remain stable in water. The difference in terms of attraction to a magnet above vs below the LCST is very marked and can ensures that extraction of the complex is efficient and very easy.

11. Immobilising BSA-Paclitaxel on Copolymer

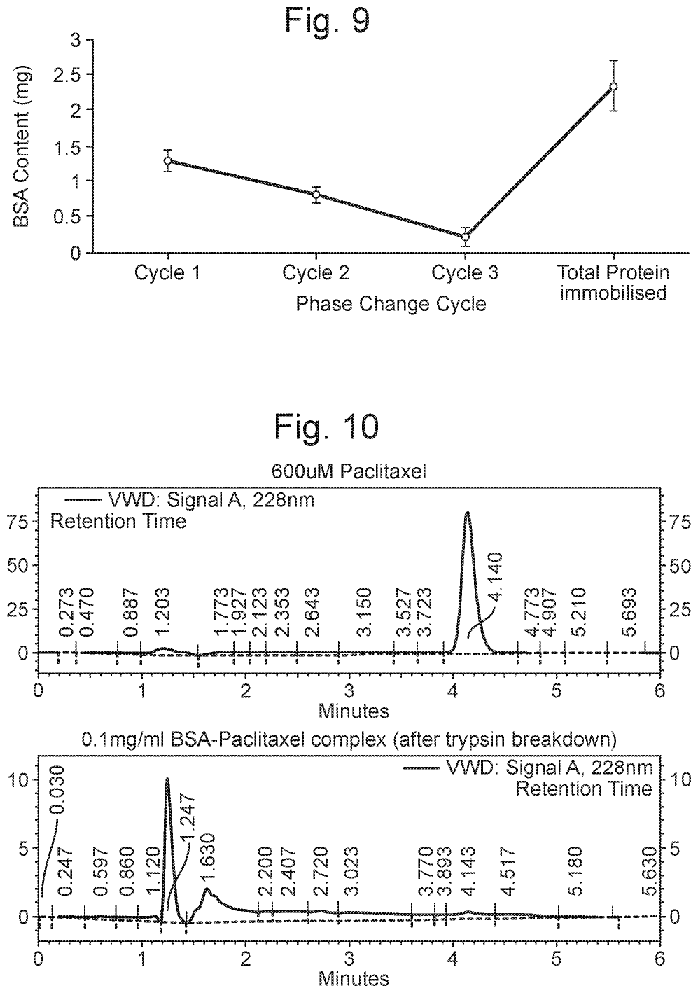

BSA (bovine serum albumin) protein was immobilised on the polyNIPAM-co-acrolein (i.e. a polymer comprising a binding unit and a phase change unit) as a means of attaching the carrier BSA to the larger nanoparticle complex. The polymer and protein were incubated at 0° C. for 24 h with rocking. After which the polymer BSA matrix was spun out and samples collected for analysis by Bradford protein assay. A “cycle” consisted of raising the polymer-BSA matrix above the LCST of the polymer and rocking for 5 mins, followed by rocking below the LCST for 5 mins and finally being precipitated out again for separation by centrifuge. The supernatant was then tested for BSA released from the polymer on each cycle. The BSA content was measured by the Bradford protein assay test and compared to a calibration curve of know protein concentration. The amount of BSA released from the polymer on each cycle is shown in FIG. 9 and the corresponding data for each of three samples tested are shown in Table 6, below.

TABLE 6
Results of the three repeats of the
BSA-polymer immobilisation test

| Sample | 1 | 2 | 3 |
|---|---|---|---|
| Supernatant | 4.14 | 3.36 | 4.08 |
| Cycle 1 | 1.15 | 1.53 | 1.22 |
| Cycle 2 | 0.66 | 0.88 | 0.93 |
| Cycle 3 | 0.14 | 0.42 | 0.12 |
| Total Protein (mg) | 1.95 | 2.83 | 2.27 |

A total weight of between 1.9-2.3 mg BSA was immobilised by 8 mg of polymer, approximately 23-35% by weight of the polymer-protein complex. This is very high as a percentage weight and proves encouraging for the prospect of immobilising BSA-PAC on iron oxide nanoparticle-bound polymers.

Subsequent variation of temperature above and below the LCST showed massive release after agitation and the induced phase change. Subsequent cycles showed the ability of the polymer to retain a large fraction of the immobilised protein through several LCST phase transition cycles. This effect could proves that the polymer may be used to release an active agent over a number of cycles.

12. Binding Paclitaxel to BSA

Paclitaxel was dissolved in anhydrous ethanol and was added to an aqueous solution of bovine serum albumin. After 90 min of equilibration at 22° C. ethanol was added and precipitated paclitaxel was removed by centrifugation, the precipitants were then dispersed in water and precipitated and isolated again for 3 cycles. The concentration of free (unbound) paclitaxel was determined from the supernatant removed from each washing cycle. The concentration of total (bound and unbound) paclitaxel in solution was then determined from the supernatant and a sample of the precipitated BSA after degradation in trypsin. Paclitaxel concentration was measured by HPLC against an internal standard and a concentration gradient. The chromatogram for this HPLC procedure is shown in FIG. 10.

The paclitaxel peaks were observed at a retention time of 4.14 minutes and were not masked by any of the breakdown products of BSA, nor the water or ethanol peaks present. BSA breakdown products show peaks 1.120, 1.247 and 1.630 minutes (FIG. 10). Paclitaxel peaks observed in the calibration gradient, internal standard and samples were all observed with the same shape and retention time. By comparing the concentration of paclitaxel detected within the particles with the content of BSA in the sample, loading was calculated and found to be 5.81% by weight, +/−0.7 wt %. The loading ratio of paclitaxel to BSA was therefore around 4:1, consistent with literature values.

When the complex comprising the BSA carrier and paclitaxel active agent was run through the disc centrifuge it is clear the BSA complexes do not form into nanoparticles. This is useful as that could complicate BSA immobilisation on polymer strands. This data demonstrates that drugs can be loaded onto a carrying vehicle at a high ratio and demonstrates the correct physiology in order to be immobilised within the larger complex.

13. Binding Vinblastine to BSA

As a demonstration of versatility of the delivery system involving a carrier, other drugs were immobilised on BSA for targeted remotely activated delivery. Vinblastine was chosen for its significant structural differences from paclitaxel and its well-established chemotherapeutic effect. Vinblastine was bound to BSA as with paclitaxel, and its release from BSA was detected using HPLC.

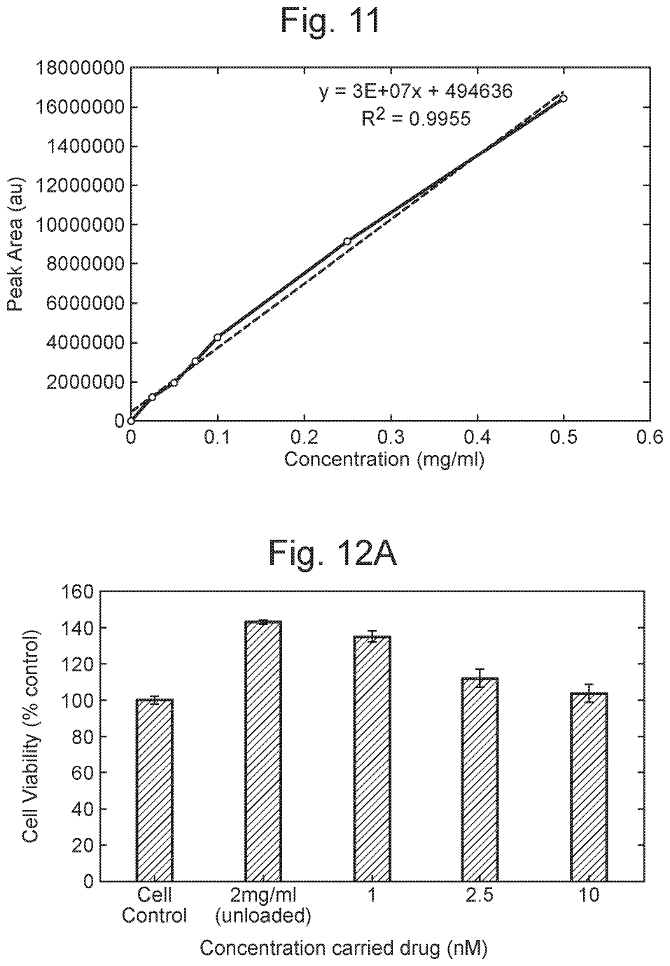

Following successful detection, a concentration gradient for vinblastine was created using a 0.5 mg/ml standard as the constant between sample measurements. The calibration curve is shown in FIG. 11. The calibration curve again was run with an internal standard and used to measure the content of Vinblastine retained by BSA. 0.28 mg of Vinblastine was calculated to have come from the 4.5 mg BSA degraded by trypsin. This gives a loading efficiency of around 30% at these ratios. The ratio of Vinblastine to BSA molecules was calculated as 5.1:1. This is similar to the loading of paclitaxel achieved on BSA (4:1). Attachment to BSA as a drug delivery vehicle for loading into the nanoparticle complex has proved versatile and is capable of delivering many active agents.

14. Binding of Paracetamol to BSA

In another example, paracetamol was bound to BSA. Paracetamol was used to demonstrate the versatility of the drug delivery system, showing that small molecules can be delivered using BSA as a carrier.

It is of interest to deliver paracetamol using the complexes described herein as a method of effectively targeting and destroying liver cancer metastasis. Liver cells show a high toxic effects when exposed to paracetamol, relative to other body tissues. Thus, there have been instances of paracetamol-induced liver failure that leads to death. This susceptibility to paracetamol is maintained in cancerous liver cells and indeed to the metastasis of liver cancer. The survival rates of liver cancer are around 7% after 5 years and part of this is due to the rapid and destructive metastasis. Paracetamol that can be selectively delivered and released to these areas of the metastasis without damaging the healthy liver tissue would provide a means to treat liver cancer patients with a greatly reduced risk.

15. Effects of Activation of Various Drug-Carrying Nanoparticle-Polymer Complex on Viability of Cell Lines.

Experiments were performed to determine whether the nanoparticle-polymer complexes described herein could retain drug molecules and effectively release them upon activation. Each experiment was repeated in triplicate on separate occasions.

Nanoparticle-polymer complexes were produced as described above using a monomer mixture comprising acrolein, acrylamide and NIPAM. BSA carrying paclitaxel or vinblastine was immobilised on the polymer. The nanoparticle-polymer complexes were then provided to various cell lines. Cells were seeded at 20×10⁴ cells per well in 24 well plates and left to incubate at 37.5° C. for 24 hours. Two such plates were set up: on one case, the nanoparticle-polymer complex was to be activated (heated) by radio waves; in the other, the complexes would remain inactivated. Following this the nanoparticle-polymer complexes were added in concentration ranges from 0.005 mg/ml up to 2 mg/ml based on the desired molar concentration of the drug to be delivered. After administration of the nanoparticle complex, the inactivated plate was placed in an incubator at 37.5° C. for 24 hours. The activated plate was placed within the induction heating coil surrounded by insulation and irradiated until the desired temperature range (40-43° C.) had been reached, the cells were then placed back in the 37.5° C. incubator for 10 minutes to equilibrate. This was repeated 3 times to ensure maximum release of drug contained in the nanoparticle complex. After these three cycles the activated plate was returned to the 37.5° C. incubator for 24 hours.

Propidium iodide staining was used to determine the alive to dead ratio of cells and therefore the cell viability after treatment with varying concentrations of the nanoparticle complex. Treated cells had their supernatant removed and stored in an Eppendorf, they were then cleaned with 200 uL PBS three times. Then 300 uL trypsin was added to mobilise viable cells, and after 10 minutes of incubation at 37.5° C. the trypsinised cells were added to same Eppendorf as the stored supernatant. The Eppendorfs were centrifuged at 1500 g for 5 minutes and the cell pellet was washed with PBS to remove any cell debris or remaining nanoparticles; this was repeated three times. Once this was complete, the cell pellets were resuspended in 50 ul PBS. 5 uL Propidium iodide stain was added and the Eppendorfs were incubated in the dark for 15 minutes. Following this the solutions were transferred to FACS tubes and a further 300 uL PBS was added.

Fluorescence activated cell sorting (FACS) was run on a FACSCalibur™. An FL3 red filter was used to pick up dead Propidium iodide stained cells and the cell count was set at a threshold of 10,000 counts. All data was collected using CellQuest™ Pro (BD Biosciences). The data was analysed using Kaluza™ 1.2.

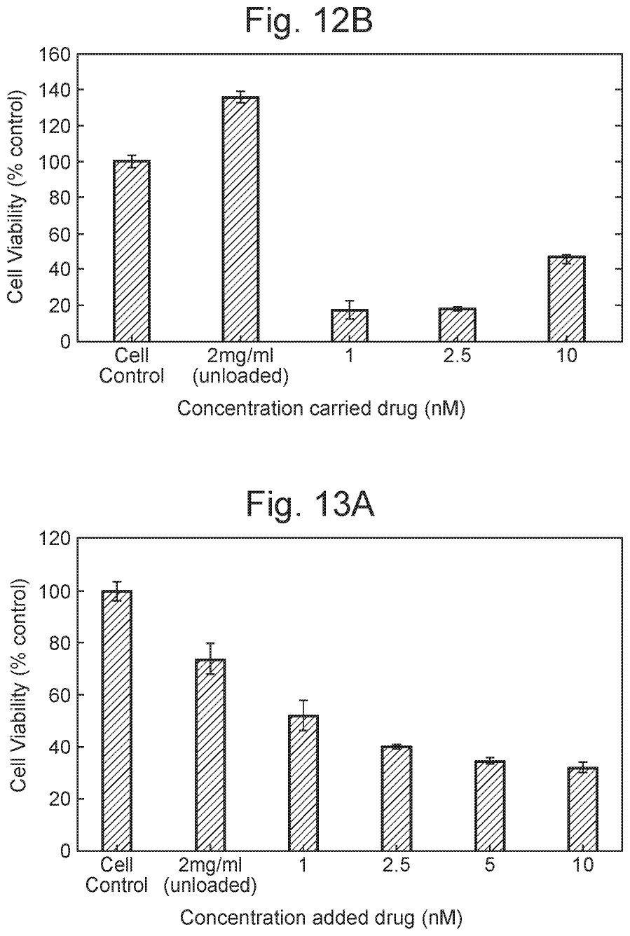

In the first experiment, complexes carrying paclitaxel were delivered to RD cell lines. The results are shown in FIG. 12A and FIG. 12B. FIG. 12A shows the outcome where the nanoparticle-polymer complexes were not activated, while FIG. 12B shows the outcome where the nanoparticle-polymer complexes were activated. It is clear that in the absence of activation, cell viability was not reduced compared to the control experiment (where no nanoparticle-polymer complex was applied). By contrast, where the complexes were activated, the percentage of viable cells was greatly decreased, indicating that paclitaxel had been released and had affected the cells. Error bars represent standard deviation of cell viability.

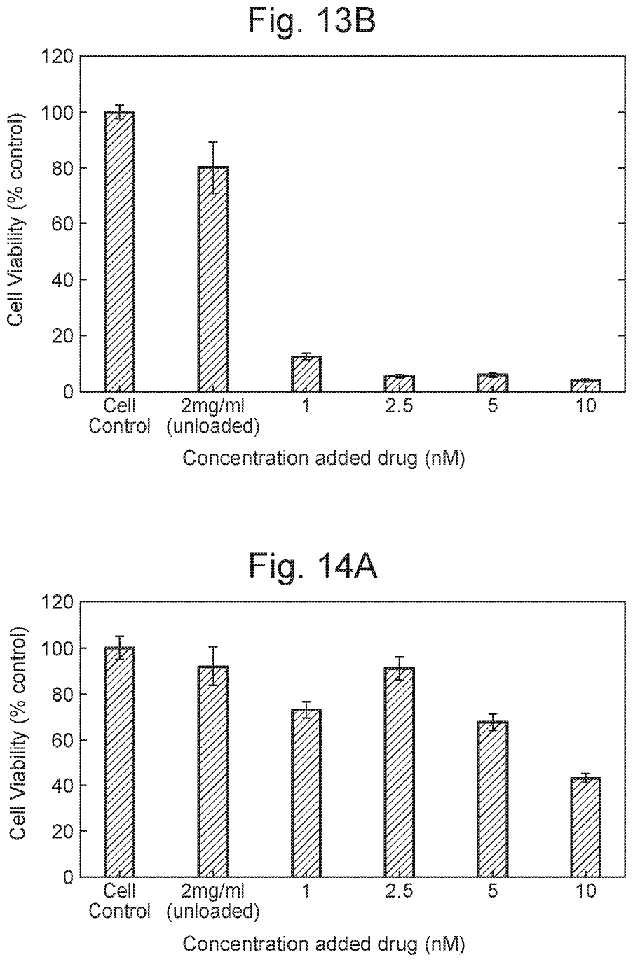

In the second experiment, the nanoparticle-polymer complexes were used to deliver paclitaxel to RH30 cells (Rhabdomyosarcoma cell lines). The results are shown in FIGS. 13A and 13B. FIG. 13A shows the outcome where the nanoparticle polymer complexes were not activated, while FIG. 13B shows the outcome where the nanoparticle polymer complexes were activated. In this case, some cell death is seen even where the complexes were not activated, but considerably greater cell death is observed where the complexes are activated.

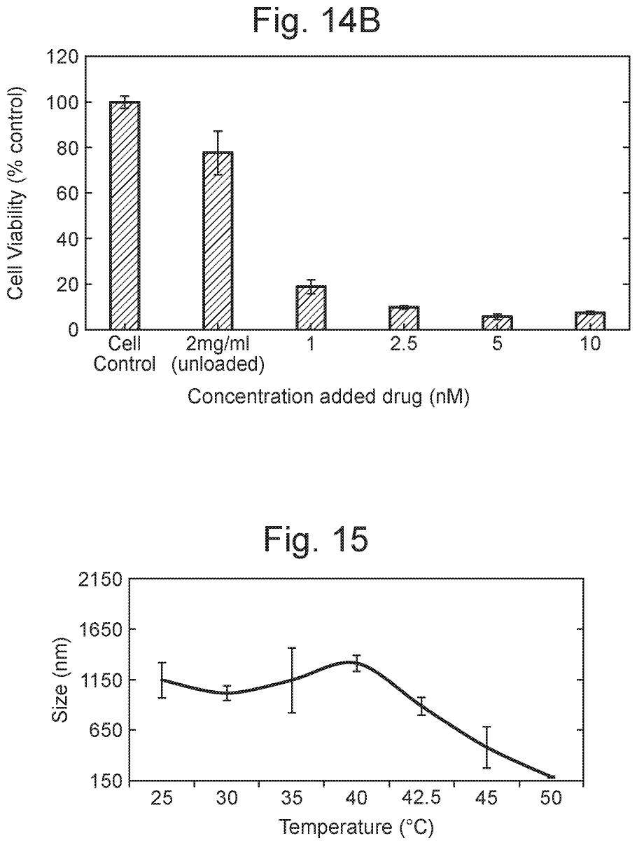

In the third experiment, the nanoparticle-polymer complexes were used to deliver vinblastine to U87 cell lines. The results are shown in FIGS. 14A and 14B. FIG. 14A shows the outcome where the nanoparticle polymer complexes were not activated, while FIG. 14B shows the outcome where the nanoparticle polymer complexes were activated. In this case, at low concentrations only activated particles caused cell death. At higher concentrations, some cell death was observed without activation but massive cell death was observed with the activated particles.

16. Effect of Variation of Composition of Thermosensitive Polymer

Thermosensitive polymers were synthesised as described above, both attached to iron oxide nanoparticles and in free form. It was found that varying the composition could vary the LCST of the polymer across a desirable range, particularly to temperatures above 40° C. Further, it was noted that the observed LCST of a polymer for any given composition appeared to be lower in a nanoparticle-polymer complex than otherwise. The results of these experiments are shown in tables 7 and 8, below.

TABLE 7
The effect of varying monomer composition on
the LCST of the copolymer formed in free solution.
Not bound to any surface or particle

| N- | | | LCST/Release |
|---|---|---|---|
| isopropylacrylamide | Acrylamide | Methacrolein | temp (° C.) |
| 50 | 30 | 20 | 27 |
| 40 | 40 | 20 | 36 |
| 35 | 45 | 20 | 43 |
| 35 | 50 | 15 | 48 |
| 40 | 50 | 10 | 50+ |

TABLE 8
The effect of varying monomer composition on the LCST of
the ION bound copolymer formed on the surface of ION.

| N- | | | LCST/Release |
|---|---|---|---|
| isopropylacrylamide | Acrylamide | Methacrolein | temp (° C.) |
| 50 | 30 | 20 | 24 |
| 40 | 40 | 20 | 31 |
| 35 | 45 | 20 | 44 |
| 35 | 50 | 15 | 38-42 |

17. Effect of Temperature on Particle Size

The effect of temperature on the nanoparticle-polymer complexes described herein was investigated. Nanoparticle-polymer complexes were produced as described above using a monomer mixture comprising acrolein, acrylamide and NIPAM. These nanoparticle-polymer complexes were made up into solutions in distilled water at concentrations of 0.1 mg/ml. These solutions were then transferred to DTS1070 disposable cuvettes before being moved to a Malvern Zetasizer Nano ZS. The solutions were heated to temperatures in the range 25-65° C. and allowed to equilibrate at the target temperature for 120 seconds. Measurements of particle size were then taken (12 repeats in total). The experiment was repeated at each temperature three times.

For magnetite particles the refractive index was set at n=1.41 and the transparency at 0.1.

The results are shown in FIG. 15. Particle size was seen to decrease at temperatures exceeding 40° C., indicating that the polymer underwent a phase transition to its globular state above this temperature.

1. A nanoparticle-polymer complex comprising an iron oxide nanoparticle bound to a polymer capable of undergoing a phase change at a predetermined temperature.

2. The nanoparticle-polymer complex according to claim 1 wherein the phase change is reversible.

3. The nanoparticle-polymer complex according to claim 1 wherein the predetermined temperature is 39° C. or more.

4. The nanoparticle-polymer complex according to claim 1 wherein the predetermined temperature is 42° C. or less.

5. The nanoparticle-polymer complex according to claim 1 wherein the polymer comprises a phase-change repeating unit of formula (XA), (XB) or (XC) 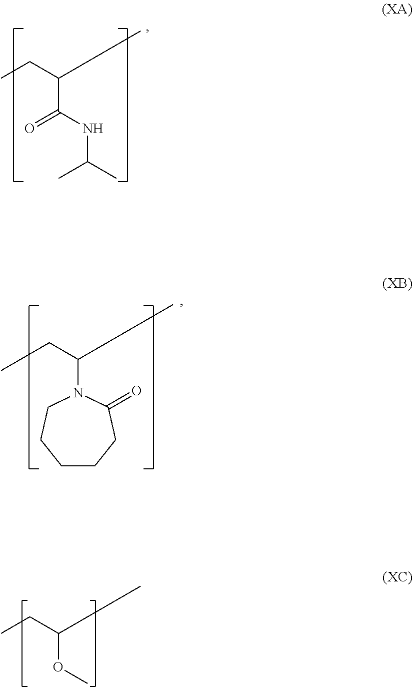
which may optionally be substituted by one, two or three substituents each independently selected from C₁₋₃ alkyl.

6. The nanoparticle-polymer complex according to claim 1 wherein the polymer comprises a nucleophilic repeating unit capable of binding to a carrier or an active agent; preferably wherein the nucleophilic repeating unit comprises an aldehyde group and/or a thiol group.

7. The nanoparticle-polymer complex according to claim 1 wherein the polymer comprises a binding unit of formula (YA), (YB), (YC), (YD) or (YE): 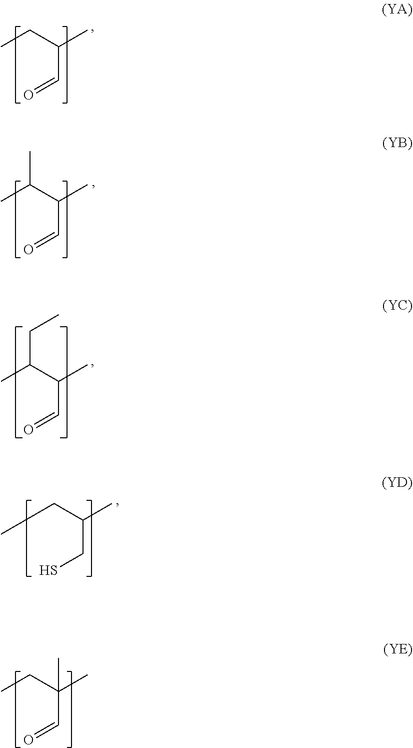
which may optionally be substituted by one, two or three substituents each independently selected from C₁₋₃ alkyl.

8. The nanoparticle-polymer complex according to claim 1 wherein the polymer comprises a LCST-adjusting repeating unit; preferably wherein the LCST-adjusting repeating unit is a hydrophilic repeating unit of formula (ZA) or (ZB): 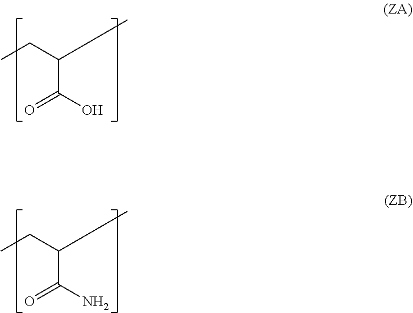
which may optionally be substituted by one, two or three substituents each independently selected from C₁₋₃ alkyl.

9. The nanoparticle-polymer complex according to claim 1 wherein the polymer comprises:
(a) a phase change repeating unit of formula (XA), a binding repeating unit of formula (YA) and a hydrophilic repeating unit of formula (ZA) 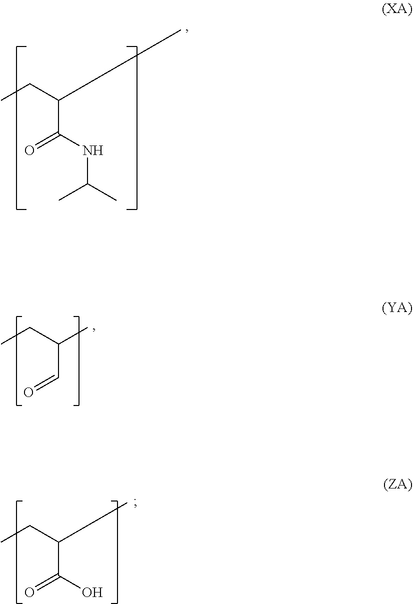
or
(b) a phase change repeating unit of formula (XA), a binding repeating unit of formula (YD) and a hydrophilic repeating unit of formula (ZB) 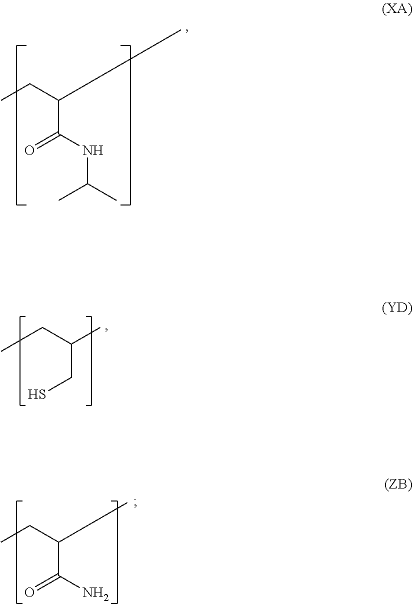
or
(c) a phase change repeating unit of formula (XA), a binding repeating unit of formula (YE) and a hydrophilic repeating unit of formula (ZB)

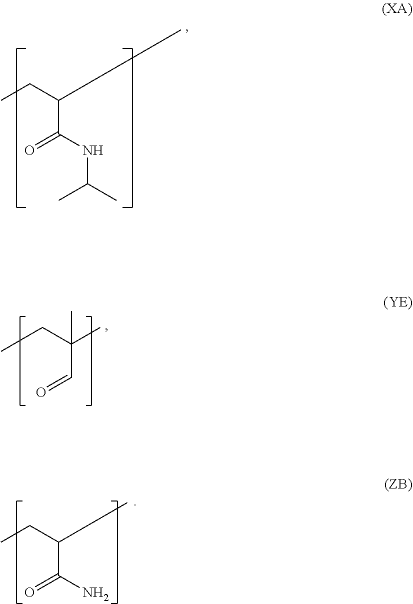

10. The nanoparticle-polymer complex according to claim 1 wherein the polymer comprises a unit of formula (I), formula (II), or formula (III): 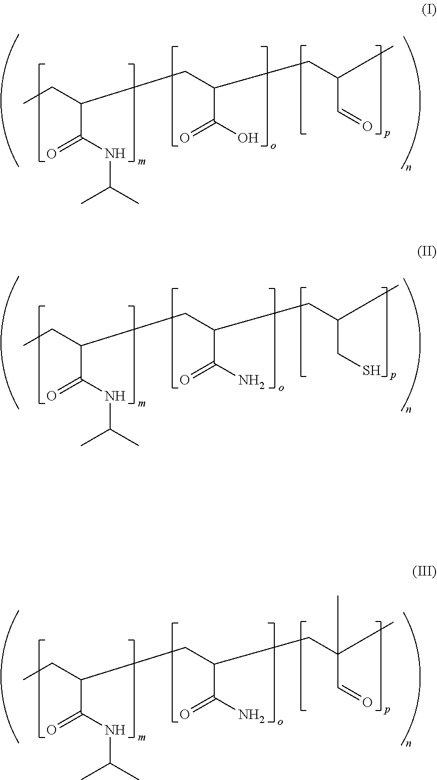
wherein n, m, o and p are integers.

11. The nanoparticle-polymer complex according to claim 1 wherein the polymer comprises an ionic anchoring group, preferably a carboxylate group.

12. The nanoparticle-polymer complex according to claim 1 wherein the nanoparticle-polymer complex further comprises a carrier immobilised on the polymer, preferably wherein the carrier is reversibly covalently bound to the polymer, most preferably wherein the carrier is bound to the polymer via a —C═N— or an —S—S— bond.

13. The nanoparticle-polymer complex according to claim 1 wherein the complex comprises a carrier which is a protein, preferably selected from bovine serum albumin or human serum albumin.

14. The nanoparticle-polymer complex according to claim 1 wherein the complex further comprises an active agent, preferably a chemotherapeutic agent.

15. The nanoparticle-polymer complex according to claim 14 wherein the active agent is selected from one or more of paclitaxel, vinblastine, paracetamol, vitamin K, vitamin C.

16. The nanoparticle-polymer complex according to claim 14 wherein the active agent is chemically bound to a carrier, which carrier is immobilised on the polymer.

17. The nanoparticle-polymer complex according to claim 1 further comprising one or more secondary active agents each independently selected from
(i) an imaging agent, preferably a fluorophore or a radio-labelled moiety; and
(ii) a targeting agent, preferably selected from a receptor-binding protein, an antibody, a nucleic acid or a peptide.

18. The nanoparticle-polymer complex according to claim 1 wherein the iron oxide nanoparticle comprises or consists of magnetite.

19. Method of releasing an active agent, which method comprises:
a) providing a nanoparticle-polymer complex comprising an active agent and an iron oxide nanoparticle bound to a polymer capable of undergoing a phase change at a predetermined temperature; and
b) exposing the nanoparticle-polymer complex to an alternating magnetic field and thereby heating the iron oxide nanoparticle, causing the phase change to occur and active agent to be released.

20-25. (canceled)

26. A thermosensitive polymer comprising a phase change repeating unit of formula (XA), a binding repeating unit of formula (YE) and a hydrophilic repeating unit of formula (ZB)

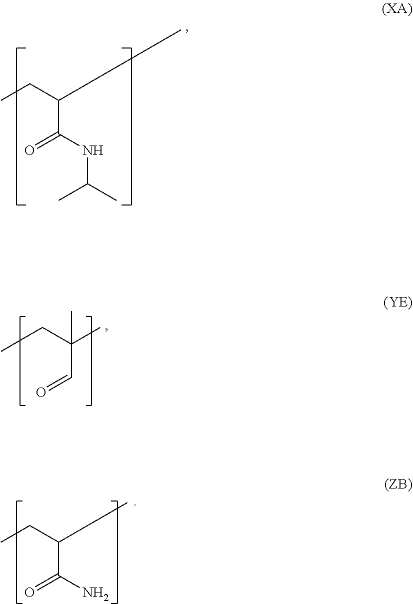

27-32. (canceled)
